(19) United States
(12) Patent Application Publication
(10) Pub. No.: US 2011/0230403 A1
(43) Pub. Date: Sep. 22, 2011

(54) GLYCOSIDE DERIVATIVES AND USES THEREOF

(75) Inventors: P. Venkata Palle, Pune (IN); Dumbala Srinivas Reddy, Andhra Pradesh (IN); Suresh Eknath Kurhade, Maharashtra (IN); Debnath Bhuniya, West Bengal (IN)

(73) Assignee: NOVARTIS AG, Basel (CH)

(21) Appl. No.: 13/063,856

(22) PCT Filed: Sep. 17, 2009
(86) PCT No.: PCT/EP2009/062054

§ 371 Date: May 12, 2011

(30) Foreign Application Priority Data
Sep. 19, 2008 (IN) 2201/DEL/08

(51) Int. Cl. A61K 38/28; C07H 7/04; A61K 31/7034; A61K 31/7064; A61K 31/7056; A61K 31/706; A61K 38/26; A61P 3/10; A61P 3/06

(52) U.S. Cl. 514/6.5; 536/116; 514/23; 536/120; 536/53; 536/54; 514/7.2

(57) ABSTRACT

The present invention relates to compounds of formula I

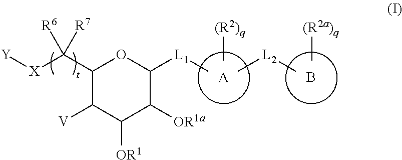

and pharmaceutically acceptable salts, to formulations and uses of the compounds of formula (I) in the treatment of metabolic disorders.

The invention relates to compounds which have an inhibitory effect on the sodium-dependent glucose cotransporter SGLT and their use in therapy.

This disclosure relates to a series of novel glycoside derivatives, their polymorphs, stereoisomers, prodrugs, solvates, pharmaceutically acceptable salts and formulations thereof. The disclosure also relates to the process for preparation of substituted glycoside derivatives along with their sodium-D-glucose cotransporter (SGLT) inhibition effects, which are beneficial for the prophylaxis, management, treatment, control of progression, or adjunct treatment of diseases and/or medical conditions where the inhibition of SGLT would be beneficial, such as diabetes (including Type-I and Type-II), obesity, dyslipidemia, insulin resistance, and other metabolic syndrome, and/or diabetes-related complications including retinopathy, nephropathy, neuropathy, ischemic heart disease, arteriosclerosis, β-cell dysfunction, and as therapeutic and/or prophylactic agents for obesity.

Diabetes mellitus is a metabolic disorder characterized by recurrent or persistent hyperglycemia (high blood glucose) and other signs, as distinct from a single disease or condition. Glucose level abnormalities can result in serious long-term complications, which include cardiovascular disease, chronic renal failure, retinal damage, nerve damage (of several kinds), microvascular damage and obesity.

Type 1 diabetes, also known as Insulin Dependent Diabetes Mellitus (IDDM), is characterized by loss of the insulin-producing β-cells of the islets of Langerhans of the pancreas leading to a deficiency of insulin. Type-2 diabetes previously known as adult-onset diabetes, maturity-onset diabetes, or Non-Insulin Dependent Diabetes Mellitus (NIDDM)—is due to a combination of increased hepatic glucose output, defective insulin secretion, and insulin resistance or reduced insulin sensitivity (defective responsiveness of tissues to insulin).

Chronic hyperglycemia can also lead to onset or progression of glucose toxicity characterized by decrease in insulin secretion from β-cell, insulin sensitivity; as a result diabetes mellitus is self-exacerbated [Diabetes Care, 1990, 13, 610]

Chronic elevation of blood glucose level also leads to damage of blood vessels. In diabetes, the resultant problems are grouped under “microvascular disease” (due to damage of small blood vessels) and “macrovascular disease” (due to damage of the arteries). Examples of microvascular disease include diabetic retinopathy, neuropathy and nephropathy, while examples of macrovascular disease include coronary artery disease, stroke, peripheral vascular disease, and diabetic myonecrosis.

Diabetic retinopathy, characterized by the growth of weakened blood vessels in the retina as well as macular edema (swelling of the macula), can lead to severe vision loss or blindness. Retinal damage (from microangiopathy) makes it the most common cause of blindness among non-elderly adults in the US. Diabetic neuropathy is characterized by compromised nerve function in the lower extremities. When combined with damaged blood vessels, diabetic neuropathy can lead to diabetic foot. Other forms of diabetic neuropathy may present as mononeuritis or autonomic neuropathy. Diabetic nephropathy is characterized by damage to the kidney, which can lead to chronic renal failure, eventually requiring dialysis. Diabetes mellitus is the most common cause of adult kidney failure worldwide. A high glycemic diet (i.e., a diet that consists of meals that give high postprandial blood sugar) is known to be one of the causative factors contributing to the development of obesity.

Type 2 diabetes is characterized by insulin resistance and/or inadequate insulin secretion in response to elevated glucose level. Therapies for type 2 diabetes are targeted towards increasing insulin sensitivity (such as TZDs), hepatic glucose utilization (such as biguanides), directly modifying insulin levels (such as insulin, insulin analogs, and insulin secretagogues), increasing incretin hormone action (such as exenatide and sitagliptin), or inhibiting glucose absorption from the diet (such as alpha glucosidase inhibitors) [Nature 2001, 414, 821-827].

Glucose is unable to diffuse across the cell membrane and requires transport proteins. The transport of glucose into epithelial cells is mediated by a secondary active cotransport system, the sodium-D-glucose cotransporter (SGLT), driven by a sodium-gradient generated by the Na+/K+-ATPase. Glucose accumulated in the epithelial cell is further transported into the blood across the membrane by facilitated diffusion through GLUT transporters [Kidney International 2007, 72, S27-S35].

SGLT belongs to the sodium/glucose cotransporter family SLCA5. Two different SGLT isoforms, SGLT1 and SGLT2, have been identified to mediate renal tubular glucose reabsorption in humans [Curr. Opinon in Investigational Drugs (2007): 8(4), 285-292 and references cited herein]. Both of them are characterized by their different substrate affinity. Although both of them show 59% homology in their amino acid sequence, they are functionally different. SGLT1 transports glucose as well as galactose, and is expressed both in the kidney and in the intestine, while SGLT2 is found exclusively in the S1 and S2 segments of the renal proximal tubule. As a consequence, glucose filtered in the glomerulus is reabsorbed into the renal proximal tubular epithelial cells by SGLT2, a low-affinity/high-capacity system, residing on the surface of epithelial cell lining in S1 and S2 tubular segments. Much smaller amounts of glucose are recovered by SGLT1, as a high-affinity/low-capacity system, on the more distal segment of the proximal tubule. In healthy human, more than 99% of plasma glucose that is filtered in the kidney glomerulus is reabsorbed, resulting in less than 1% of the total filtered glucose being excreted in urine. It is estimated that 90% of total renal glucose absorption is facilitated by SGLT2; remaining 10% is likely mediated by SGLT1 [J. Parenter. Entert Nutr. 2004, 28, 364-371].

The SGLT2 was cloned as a candidate sodium glucose cotransporter, and its tissue distribution, substrate specificity, and affinities are reportedly very similar to those of the low-affinity sodium glucose co-transporter in the renal proximal tubule. A drug with a mode of action of SGLT2 inhibition will be a novel and complementary approach to existing classes of medication for diabetes and its associated diseases to meet the patient's needs for both blood glucose control, while preserving insulin secretion. In addition, SGLT2 inhibitors which lead to loss of excess glucose thereby excess calorie may have additional potential for the treatment of obesity.

Indeed small molecule SGLT2 inhibitors have been discovered and antidiabetic therapeutic potential of such molecules have been reported in literature [T-1095 (Diabetes, 1999, 48, 1794-1800, Dapagliflozin (Diabetes, 2008, 57, 1723-1729)]. Various O-aryl and O-heteroaryl glycosides have been reported as SGLT-2 inhibitors in patent publications such as: WO 01/74834, WO 03/020737, US 04/0018998, WO 01/68660, WO 01/16147, WO 04/099230, WO 05/011592, US 06/0293252, WO 05/021566.

Various glucopyranosyl-substituted aromatic and heteroaromatic compounds have also been reported as SGLT-2 inhibitors in patent publications such as: WO 01/27128, WO 04/080990, US 06/0025349, WO 05/085265, WO 05/085237, WO 06/054629, WO 06/011502.

SGLT1 is predominantly found in the intestine and plays a major role in the absorption of D-glucose and D-galactose. Therefore, SGLT1 inhibitors have the potential to act both in the kidney as well as the intestine to reduce calorie intake and hyperglycemia.

WO2004/018491 discloses pyrazole derivatives which are SGLT1 inhibitors.

Glucopyranosyl-substituted aromatic or heteroaromatic compounds where, in general, the sugar moiety has been modified at C4, C5, or C6 positions of pyranose have been published (US 06/0009400, US 06/0019948, US 06/0035841, US 06/0074031, US 08/002,7014, WO 08/016,132).

For the purposes of this invention inhibition of SGLT means inhibitions exclusively of SGLT2, inhibitions exclusively of SGLT1 or inhibition of both SGLT1 and SGLT2.

Thus, as a first embodiment, the invention provides a compound of formula I:

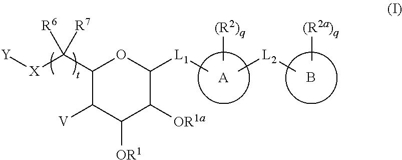

or a pharmaceutically acceptable salt thereof, wherein
Rings A and B are independently C₆₋₁₀aryl, C₃₋₇cycloalkyl, heteroaryl or heterocyclic;
L₁ is —S(O)ₚ—, —N(R³)—, or —(CH₂)ₙ—, provided that L₁ is not —N(R³)— when X is —O—;
L₂ is —(CH₂)ₙO(CH₂)ₘ—, —S(O)ₚ—, —N(R³)—, —Si(R′)(R″)—, —(C(R′)(R″))ₙ—, —(CH₂)ₙC(O)(CH₂)ₘ—, —(CH₂)ₙC(O)NR³(CH₂)ₘ—, —(CH₂)ₙNR³C(O)(CH₂)ₘ, —C₂₋₆alkenyl-, —C(O)C₂₋₆alkenyl-, —N(R)C(O)N(R³)—, —N(R³)SO₂—, or —SO₂N(R³)—;
V is halogen, OR^{1b} or hydrogen;
with the proviso that, when V is —OR^{1b}, Y is C₆₋₁₀aryl, L₁ is bond, L₂ is —CH₂—, rings A and B are phenyl, then Y is not unsubstituted aryl or an aryl that is substituted exclusively with halogen, C₁₋₆haloalkyl, C₁₋₆perhaloalkyl, C₁₋₆alkoxy, C₉₋₆haloalkoxy, C₁₋₆perhaloalkoxy or cyano as substituents;
t is an integer from 1-4;
m, for each occurrence, is independently, 0 or an integer from 1-4;
n, for each occurrence, is independently, 0 or an integer from 1-4;
p, for each occurrence, is independently, 0 or an integer from 1-2;
R′ and R″, for each occurrence, are independently hydrogen, halogen, C₁₋₆alkyl, or C₁₋₆perhaloalkyl or taken together form a cyclic ring which may optionally have heteroatoms selected from O, N or S;
R¹, R^{1a} and R^{1b} are independently selected from hydrogen, C₁₋₆alkyl, C₆₋₁₀arylC₁₋₄alkyl, —C(O)C₆₋₁₀aryl or —C(O)C₁₋₆alkyl;
R² and R^{2a}, for each occurrence, are independently halogen, hydroxy, C₁₋₄hydroxylalkyl, cyano, —NR⁴R⁵, —CH₂NR⁴R⁵, C₁₋₄alkyl, C₃₋₇cycloalkyl, C₁₋₄alkoxy, C₃₋₇cycloalkoxy, —S(O)ₚR³, —S(O)₂NR⁴R⁵, —OS(O)₂R³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₃₋₇cycloalkyl, C₃₋₇cyoloalkylC₁₋₄alkyl, C₆₋₁₀aryl, C₆₋₁₀arylC₁₋₄alkyl, C₈₋₁₀aryloxy, heterocyclyl, heterocyclylC₁₋₄alkyl, heteroaryl, heteroatylC₁₋₄alkyl, heteroaryloxy, or heterocycloxy;
R³ is hydrogen, C₁₋₆alkyl, C₃₋₇ cycloalkyl, C₆₋₁₀aryl, heteroaryl, —NR⁴R⁵ or heterocyclyl;
q, for each occurrence, is independently 0 or an integer from 1-3;
Y is C₆₋₁₀aryl, C₃₋₇cycloalkyl, heteroaryl, or heterocyclic, each of which may be optionally substituted;

### X is S(O)ₚ or O;

R⁴ and R⁵, for each occurrence, are each independently hydrogen, C₁₋₆alkyl, C₃₋₇ cycloalkyl, C₃₋₇cycloalkylC₁₋₄alkyl, C₆₋₁₀arylC₁₋₄alkyl, C₆₋₁₀aryl, heteroaryl, heteroarylC₁₋₄alkyl, heterocyclyl, or heterocyclylC₁₋₄alkyl, or
R⁴ and R⁵ taken together may form a monocyclic or a bicyclic ring system which may be saturated, partially saturated or aromatic and may optionally have additional heteroatoms selected from O, N or S, the said ring system may further be optionally substituted;
R⁶ and R⁷, for each occurrence, are independently hydrogen, C₁₋₆alkyl, C₁₋₆ hydroxyalkyl, C₆₋₁₀aryl, C₆₋₁₀arylC₁₋₄alkyl, C₃₋₇cycloalkyl, heterocyclyl, heterocyclylC₁₋₄alkyl, heteroaryl or heteroarylC₁₋₄alkyl; or
R⁶ and R⁷ taken together may form a spiro, monocyclic or a bicyclic ring system which may be saturated or partially saturated and may optionally have additional heteroatoms selected from O, N or S, the said ring system may further be optionally substituted; wherein when a group is optionally substituted, the substituents are selected from the group consisting of hydroxyl, cyano, nitro, C₁₋₆alkyl, C₂₋₆alkenyl, C₂₋₆alkynyl, C₁₋₆alkoxy, C₂₋₆alkenyloxy, C₂₋₆alkynyloxy, halogen, C₁₋₆haloalkyl, C₁₋₆perhaloalkyl, C₁₋₆₋alkylcarbonyl, (CH₂)ₙ—COOR³, amino, C₁₋₆₋alkylamino, di-C₁₋₆₋alkylamino, aminocarbonyl, C₁₋₆₋alkylaminocarbonyl, di-C₁₋₆₋alkylaminocarbonyl, C₁₋₆₋alkylcarbonylamino, C₁₋₆₋alkylcarbonyl(C₁₋₆₋alkyl)amino, C₁₋₆alkoxycarbonylamino, C₁₋₆₋alkylsulfonylamino, C₁₋₆₋alkylsulfonyl(C₁₋₆₋alkyl)amino, C₁₋₆alkylthio, C₁₋₆₋alkylsulfanyl, C₁₋₆₋alkylsulfinyl, C₁₋₆₋alkylsulfonyl, aminosulfonyl, C₁₋₆₋alkylaminosulfonyl and di-C₁₋₆alkylaminosulfonyl, aminocarbonylC₁₋₆alkyl, C₁₋₆alkylaminocarbonylC₁₋₆alkyl, di-C₁₋₆alkylaminocarbonylC₁₋₆alkyl, sulfanylC₁₋₆alkyl, C₁₋₆alkylsulfanylC₁₋₆alkyl, sulfinylC₁₋₆alkyl, C₁₋₆alkylsulfinylC₁₋₆alkyl, sulfonylC₁₋₆alkyl, C₁₋₆alkylsulfonylC₁₋₆alkyl, cycloalkyl, C₆₋₁₀aryl (such as a phenyl), heterocyclyl, heteroaryl, heterocyclylcarbonyl, pyrrolidinocarbonyl, azetidinocarbonyl, cycloalkylcarbonylamino, cyclopropylcarbonylamino, cyclopentycarbonylamino, cyclohexylcarbonylamino, C₆₋₁₀arylcarbonylamino, and phenylcarbonylamino, wherein each of the aforementioned groups may be optionally substituted by one or more halogen, C₁₋₆alkyl, hydroxyl, oxo, C₁₋₆₋alkoxy, amino, C₁₋₆₋alkylamino, di-C₁₋₆₋alkylamino or cyano.

For purposes of interpreting this specification, the following definitions will apply and whenever appropriate, terms used in the singular will also include the plural and vice versa.

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

“Alkylene” refers to a straight or branched divalent hydrocarbon chain consisting solely of carbon and hydrogen atoms, having from one to twelve carbon atoms, preferably one to 6 carbon atoms, and linking the rest of the molecule to a radical group. Examples of alkylene groups include methylene, ethylene, propylene, n-butylene, and the like. The alkylene is attached to the rest of the molecule through a single bond and to the radical group through a single bond. The points of attachment of the alkylene to the rest of the molecule and to the radical group can be through one carbon or any two carbons within the chain. In one embodiment, an alkylene group may be optionally substituted by one or more of the following groups: C₁₋₄ alkyl, trihaloC₁₋₄alkyl, halogen, or hydroxyl.

As used herein, the term “haloalkyl” refers to an alkyl, as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro substituent. Dihaloalky and polyhaloalkyl groups can be substituted with two or more of the same halo atoms or a combination of different halo groups. Preferably, a polyhaloalkyl is substituted with up to 12, 10, 8, 6, 4, 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

“Halogen” or “halo” may be fluoro, chloro, bromo or iodo.

As used herein, the term “hydroxyalkyl” refers to an alkyl, as defined herein, that is substituted by one or more hydroxy groups. Preferably the hydroxyalkyl can be monohydroxyalkyl or dihydroxyalkyl. Non-limiting examples of hydroxyalkyl include 2-hydroxyethyl, 3-hydroxypropyl, 2-hydroxypropyl, and the like.

The term “alkenyl” refers to a monovalent hydrocarbon having at least one carbon-carbon double bond. The term “C₂-C₆alkenyl” refers to a monovalent hydrocarbon having two to six carbon atoms and comprising at least one carbon-carbon double bond.

The term “alkynyl” refers to a monovalent hydrocarbon having at least one carbon-carbon triple bond. The term “C₂-C₆-alkynyl” refers to a monovalent hydrocarbon having two to six carbon atoms and comprising at least one carbon-carbon triple bond.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Preferably, alkoxy groups have about 1-6, more preferably about 1-4 carbons.

As used herein, the term “haloalkoxy” refers to haloalkyl-O—, wherein haloalkyl is defined herein above. A representative example of a haloalkoxy is 1,2-dichloroethoxy. Preferably, haloalkoxy groups have about 1-6, more preferably about 1-4 carbons.

As used herein, the term “perhaloalkoxy” refers to perhaloalkyl-O—, wherein perhaloalkyl is defined herein above. A representative example of a haloalkoxy is trifluoromethoxy. Preferably, perhaloalkoxy groups have about 1-6, more preferably about 1-4 carbons.

Alkyl, alkenyl, alkynyl, and alkoxy groups, containing the requisite number of carbon atoms, can be unbranched or branched. The requisite number of carbon may be represented as C₁₋₆, C₁₋₄, etc.

The term “aryl” refers to monocyclic or bicyclic aromatic hydrocarbon groups having 6-10 carbon atoms in the ring portion. Non-limiting examples include phenyl and naphthyl, each of which may optionally be substituted by 1-4 substituents, such as C₁₋₆alkyl, trifluoromethyl, C₃₋₇cycloalkyl, halogen, hydroxy, C₁₋₆alkoxy, acyl, C₁₋₆alkyl-C(O)—O—, C₆₋₁₀aryl-O—, heteroaryl-O—, amino, thiol, C₁₋₆alkyl-S—, C₆₋₁₀aryl-S—, nitro, cyano, carboxy, C₁₋₆alkyl-O—C(O)—, carbamoyl, C₁₋₆alkyl-S(O)—, sulfonyl, sulfonamido, or heterocyclyl.

The term “aryl” also refers to a bicyclic group in which a monocyclic aryl ring is fused to one or more or heterocyclyl rings or cycloalkyl rings, where the radical or point of attachment is on the aryl ring. Nonlimiting examples include tetrahydronaphthylene, indane, benzoxazine, and chroman.

As used herein, the term “acyl” refers to a group R—C(O)—, wherein R in the acyl residue is C₁₋₆alkyl, or C₁₋₆alkoxy, or C₆₋₁₀aryl, or heteroaryl. Also preferably, one or more carbons in the acyl residue may be replaced by nitrogen, oxygen or sulfur as long as the point of attachment to the parent remains at the carbonyl. Examples of acyl include but are not limited to, acetyl, benzoyl, propionyl, isobutyryl, t-butoxycarbonyl, benzyloxycarbonyl and the like. Lower acyl refers to acyl containing one to four carbons.

As used herein, the term “carbamoyl” refers to H₂NC(O)—, C₁₋₆alkyl-NHC(O)—, (C₁₋₆alkyl)₂NC(O)—, C₆₋₁₀aryl-NHC(O)—, C₁₋₆alkyl(C₆₋₁₀aryl)-NC(O)—, heteroaryl-NHC(O)—, C₁₋₆alkyl(heteroaryl)-NC(O)—, C₆₋₁₀aryl-C₁₋₆alkyl-NHC(O)—, or C₁₋₆alkyl(C₆₋₁₀aryl-C₁₋₆alkyl)-NC(O)—.

As used herein, the term “sulfonyl” refers to R—SO₂—, wherein R is hydrogen, C₁₋₆alkyl, C₆₋₁₀aryl, hereoaryl, C₆₋₁₀aryl-C₁₋₆alkyl, heteroaryl-C₁₋₆alkyl, C₁₋₆alkoxy, C₆₋₁₀aryloxy, C₃₋₇cycloalkyl, or heterocyclyl.

As used herein, the term “sulfonamido” refers to C₁₋₆alkyl-S(O)₂—NH—, C₆₋₁₀aryl-S(O)₂—NH—, C₆₋₁₀aryl-C₁₋₆alkyl-S(O)₂—NH—, heteroaryl-S(O)₂—NH—, heteroaryl-C₁₋₆alkyl-S(O)₂—NH—, C₁₋₆alkyl-S(O)₂—N(C₁₋₆alkyl)-, C₆₋₁₀aryl-S(O)₂—N(C₁₋₆alkyl)-, C₆₋₁₀aryl-C₁₋₆alkyl-S(O)₂—N(C₁₋₆alkyl)-, heteroaryl-S(O)₂—N(C₁₋₆alkyl)-, or heteroaryl-C₁₋₆alkyl-S(O)₂—N(C₁₋₆alkyl)-.

As used herein, the term “sulfamoyl” refers to (R)₂NSO₂—, wherein R, for each occurrence is independently hydrogen, C₁₋₆alkyl, C₆₋₁₀aryl, hereoaryl, C₆₋₁₀aryl-C₁₋₆alkyl, heteroaryl-C₁₋₆alkyl, C₁₋₆alkoxy, C₆₋₁₀aryloxy, C₃₋₇cycloalkyl, or heterocyclyl.

As used herein, the term “heterocyclyl” or “heterocyclo” refers to an optionally substituted, saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include dihydrofuranyl, [1,3]dioxolane, 1,4-dioxane, 1,4-dithiane, piperazinyl, 1,3-dioxolane, imidazolidinyl, imidazolinyl, pyrrolidine, dihydropyran, oxathiolane, dithiolane, I,3-dioxane, 1,3-dithianyl, oxathianyl, thiomorpholinyl, oxiranyl, aziridinyl, oxetanyl, azetidinyl, tetrahydrofuranyl, pyrrolidinyl, tetrahydropyranyl, piperidinyl, morpholinyl, piperazinyl, azepinyl, oxapinyl, oxazepinyl and diazepinyl.

In one embodiment, a heterocyclyl may be substituted with 1, 2 or 3 substituents selected from the groups consisting of the following:
(a) C₁₋₆alkyl;
(b) hydroxy (or protected hydroxy);
(c) halo;
(d) oxo, i.e., ═O;
(e) amino (i.e. NH₂), C₁₋₆alkylamino or di-(C₁₋₆alkyl)amino;
(f) C₁₋₆alkoxy;
(g) C₃₋₇cycloalkyl;
(h) carboxyl;
(i) heterocyclooxy, wherein heterocyclooxy denotes a heterocyclic group bonded through an oxygen bridge;
(j) C₁₋₆alkyl-O—C(O)—;
(k) mercapto;
(l) nitro;
(m) cyano;
(n) sulfamoyl or sulfonamido;
(o) C₆₋₁₀aryl;
(p) C₁₋₆alkyl-C(O)—O—;
(q) C₆₋₁₀aryl-C(O)—O—;
(r) C₆₋₁₀aryl-S—;
(s) C₆₋₁₀aryloxy;
(t) C₁₋₆alkyl-S—;
(u) formyl i.e., HC(O)—;
(v) carbamoyl;
(w) C₆₋₁₀aryl-C₁₋₆alkyl-; and
(x) C₆₋₁₀aryl substituted with C₁₋₆alkyl, C₃₋₇cycloalkyl, C₁₋₆alkoxy, hydroxy, amino, C₁₋₆alkyl-C(O)—NH—, C₁₋₆alkylamino, di-(C₁₋₆alkyl)amino or halogen.

As used herein, the term “heterocyclylalkyl” is a heterocyclyl as defined above which is attached to another moiety through an alkylene group, e.g. morpholine-CH₂—.

As used herein, the term “cycloalkyl” refers to saturated or partially unsaturated (but not aromatic) monocyclic, bicyclic or tricyclic hydrocarbon groups of 3-12 carbon atoms, preferably 3-9, or 3-7 carbon atoms, each of which can be optionally substituted by one, or two, or three, or more substituents, such as C₁₋₆alkyl, halo, oxo, hydroxy, C₁₋₆alkoxy, C₁₋₆alkyl-C(O)—, carbamoyl, C₁₋₆alkyl-NH—, (C₁₋₆alkyl)₂N—, thiol, C₁₋₆alkyl-S—, nitro, cyano, carboxy, C₁₋₆alkyl-O—C(O)—, sulfonyl, sulfonamido, sulfamoyl, or heterocyclyl. Exemplary monocyclic hydrocarbon groups include, but are not limited to, cyclopropyl, cyclobutyl, cyclopentyl, cyclopentenyl, cyclohexyl or cyclohexenyl. Exemplary bicyclic hydrocarbon groups include bornyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, or bicyclo[2.2.2]octyl. Exemplary tricyclic hydrocarbon groups include adamantyl.

As used herein, the term “aryloxy” refers to an —O-aryl, wherein aryl is defined herein.

As used herein, the term “heteroaryloxy” refers to an —O-heteroaryl, wherein heteroaryl is defined herein.

As used herein, the term “heteroaryl” refers to a 5-14 membered monocyclic- or bicyclic-aromatic ring system, having 1 to 8 heteroatoms selected from N, O or S. Preferably, the heteroaryl is a 5-10 or 5-7 membered ring system. Examples of monocyclic heteroaryl groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. Examples of bicyclic heteroaryl groups include indolyl, benzofuranyl, quinolyl, isoquinolyl indazolyl, indolinyl, isoindolyl, indolizinyl, benzamidazolyl, and quinolinyl. More specific heteroaryl groups include 2- or 3-thien-2-yl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more cycloalkyl, or heterocyclyl rings, where the radical or point of attachment is on the heteroaromatic ring. Nonlimiting examples include 5,6,7,8-tetrahydroquinoline and 6,7-dihydro-5H-pyrrolo[3,2-d]pyrimidine.

A heteroaryl group may be mono-, bi-, tri-, or polycyclic, preferably mono-, bi-, or tricyclic, more preferably mono- or bicyclic.

“Heteroaryl” and “heterocyclyl” is also intended to include oxidized S or N, such as sulfinyl, sulfonyl and N-oxide of tertiary ring nitrogen.

When an alkyl, alkenyl, alkoxy, cycloalkyl, aryl, arylalkyl, heteroaryl, heterocyclyl, heterocyclylalkyl is optionally substituted, it may be substituted with one or more than one substituents selected from hydroxyl, cyano, nitro, C₁₋₆₋alkyl, C₂₋₆₋alkenyl, C₂₋₆₋alkynyl, C₁₋₆-alkoxy, C₂₋₆₋alkenyloxy, C₂₋₆₋alkynyloxy, halogen, C₁₋₆haloalkyl, C₁₋₆perhaloalkyl, C₁₋₆alkylcarbonyl, (CH₂)ₙ—COOR³, amino, C₁₋₆₋alkylamino, di-C₁₋₆₋alkylamino, C₁₋₆₋alkylaminocarbonyl, di-C₁₋₆₋alkylaminocarbonyl, C₁₋₆₋alkylcarbonylamino, C₁₋₆₋alkylcarbonyl(C₁₋₆₋alkyl)amino, C₁₋₆₋alkylsulfonylamino, C₁₋₆₋alkylsulfonyl(C₁₋₆₋alkyl)amino, C₁₋₆₋alkylthiol, C₁₋₆₋alkylsulfanyl, C₁₋₆₋alkylsulfinyl, aminosulfonyl, C₁₋₆₋alkylaminosulfonyl and di-C₁₋₆alkylaminosulfonyl, aminocarbonylC₁₋₆alkyl, C₁₋₆aminocarbonylC₁₋₆alkyl, di-C₁₋₆aminocarbonylC₁₋₆alkyl, sulfanylC₁₋₆alkyl, C₁₋₆alkylsulfanylC₁₋₆alkyl, sulfinylC₁₋₆alkyl, C₁₋₆alkylsulfinylC₁₋₆alkyl, sulfonylC₁₋₆alkyl, C₁₋₆alkylsulfonylC₁₋₆alkyl, C₃₋₇cycloalkyl, C₆₋₁₀aryl, heterocyclyl, heteroaryl, where each of the aforementioned hydrocarbon groups may be optionally substituted by one or more halogen, C₁₋₆alkyl, hydroxyl, oxo, C₁₋₆₋alkoxy, amino, C₁₋₆₋alkylamino, di-C₁₋₆₋alkylamino or cyano.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood or conversion in the gut or liver. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)).

A discussion of prodrugs is provided in Higuchi, T., et al., “Pro-drugs as Novel Delivery Systems,” A. C. S. Symposium Series, Vol. 14, and in Bioreversible Carriers in Drug Design, ed. Edward B. Roche, Anglican Pharmaceutical Association arid Pergamon Press, 1987.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphorirc acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients thereof.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Unless otherwise indicated, the present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

The present invention includes all pharmaceutically acceptable isotopically-labeled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁵Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Isotopically-labeled compounds of Formula (I) can generally be prepared by conventional techniques known to those skilled in the art or by processes analogous to those described in the accompanying Examples and Preparations Sections using an appropriate isotopically-labeled reagent in place of the non-labeled reagent previously employed.

Various embodiments of the invention are described below. It will be appreciated that the features specified in each embodiment may be combined with other specified features, to provide further embodiments.

In further or alternative embodiment of the present invention, compounds may be represented by formula (II) or (III)

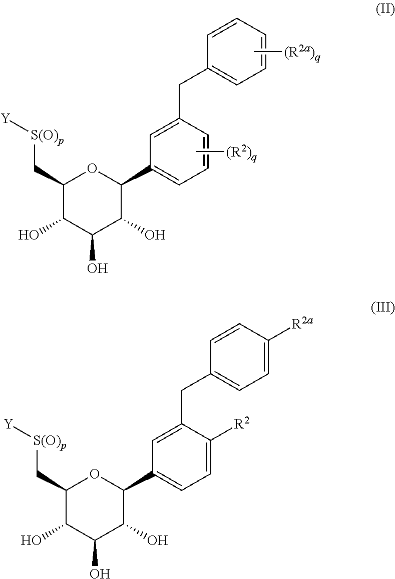

or a pharmaceutically acceptable salt thereof, wherein,
R² and R^{2a}, for each occurrence, are independently selected from halogen, hydroxy, C₁₋₄ hydroxylalkyl, cyano, —NR⁴R⁵, —CH₂NR⁴R⁵, C₁₋₄ alkyl, C₃₋₇ cycloalkyl, C₁₋₄ alkoxy, —S(O)ₚR³, —OS(O)ₚR³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₆₋₁₀aryloxy, heterocyclyl and heteroaryl;
p, for each occurrence, is independently 0, 1 or 2;
q, for each occurrence, is independently 1, 2, or 3; and
Y is optionally substituted C₆₋₁₀aryl or an optionally substituted heteroaryl.

In further or alternative embodiment of the present invention, compounds may be represented by formula (IV) or (V)

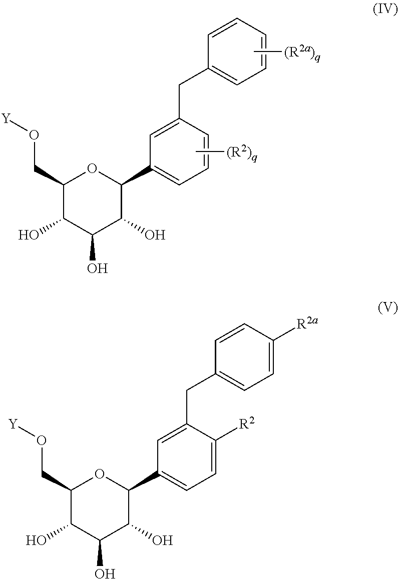

or a pharmaceutically acceptable salt thereof, wherein,
R² and R^{2a}, for each occurrence, are independently selected from halogen, hydroxy, C₁₋₄ hydroxylalkyl, cyano, —NR⁴R⁵, —CH₂NR⁴R⁵, C₁₋₄ alkyl, C₃₋₇ cycloalkyl, C₁₋₄ alkoxy, —S(O)ₚR³, —OS(O)ₚR³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₆₋₁₀aryloxy, heterocyclyl and heteroaryl;
p, for each occurrence, is independently 0, 1 or 2;
q, for each occurrence, is independently 1, 2, or 3; and
Y is optionally substituted C₆₋₁₀aryl or an optionally substituted heteroaryl.

References hereinafter to compounds of formula (I) apply equally to compounds of formula (II), (III), (IV) and (V).

References hereinafter to embodiments of the invention apply equally to compounds of formula (I) and compounds of formula (II), (III), (IV) and (V), insofar as the embodiments are present.

Various embodiments of the invention are described below. It will be appreciated that the feature specified in each embodiment may be combined with other specified features, to provide further embodiments.

In one embodiment, rings A and B are phenyl.

In another embodiment, X is O.

In another embodiment, X is S(O)ₚ.

In another embodiment, R⁶ and R⁷ taken together can form a cyclic ring, which may optionally have heteroatoms selected from O, N or S, Non limitative examples of such spiro cyclic systems are

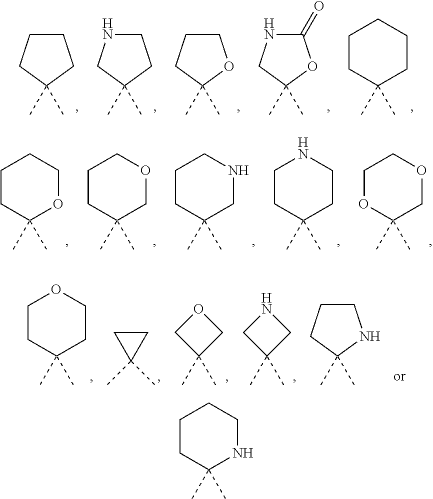

In another embodiment, L₁ is a bond.

In another embodiment, L₂ is —(CH₂)—.

In another embodiment, V is halogen, e.g. fluoro. In a further embodiment, V is —OH.

In another embodiment, R¹ and R^{1a} are hydrogen.

In another embodiment, R² is halogen, e.g. chloro.

In another embodiment, R^{2a} is C₁ alkoxy, e.g. methoxy or ethoxy.

In another embodiment, q=1.

In another embodiment, rings A and B are phenyl, L1 is a bond, L2 is —(CH₂)—, V is —OH, R¹ and R^{1a} are hydrogen, R² is chloro, R^{2a} is ethoxy and q=1.

In another embodiment of the invention, Y is C₆₋₁₀aryl or heteroaryl.

In another embodiment of the invention, Y is a substituted phenyl group.

In another embodiment of the invention, Y is

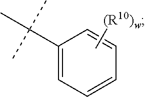

R¹⁰ is independently halogen, hydroxy, C₁₋₄ hydroxylalkyl, cyano, —NR¹⁶R¹⁷, oxo (═O), —CH₂NR¹⁶R¹⁷, C₁₋₄ alkyl, C₃₋₇ cycloalkyl, C₁₋₄alkoxy, —S(O)ₚR¹⁸, —OS(O)₂R¹⁸, —C(O)R¹⁸, —C(O)OR¹⁸, —CH₂C(O)OR¹⁸, —C(O)NR¹⁶R¹⁷, —CH₂C(O)NR¹⁶R¹⁷, —NR¹⁸C(O)NR¹⁶R¹⁷, —NR¹⁸C(O)R¹⁸, —NR¹⁸C(O)OR¹⁸, —CH₂NR¹⁶C(O)OR¹⁸, —CH₂NR¹⁶C(O)NR¹⁶R¹⁷, —CH₂NR¹⁶S(O)ₚR¹⁸, —S(O)₂NR¹⁶R¹⁷, C₁₋₆ haloalkyl, C₁₋₆ perhaloalkyl, C₆₋₁₀aryloxy, heterocyclyl, heteroaryl;
R¹⁸ is hydrogen, C₁₋₆ alkyl, C₃₋₇ cycloalkyl, C₆₋₁₀aryl, heteroaryl, or heterocyclyl;
R¹⁶ and R¹⁷ are independently hydrogen, C₁₋₆ alkyl, C₃₋₇ cycloalkyl, C₆₋₁₀aryl(C₁₋₄)alkyl, C₆₋₁₀aryl, heteroaryl, heteroaryl(C₁₋₄)alkyl, heterocyclyl, heterocyclyl(C₁₋₄alkyl or
R¹⁶ and R¹⁷ taken together may form a monocyclic or a bicyclic ring system which may be saturated, partially saturated or aromatic and may optionally have additional heteroatoms selected from O, N or S, the said ring system may further be optionally substituted;
   p, for each occurrence, is independently 0, 1 or 2; and
   w is 0, or an integer from 1-4.

In another embodiment, R¹⁰ is halogen, e.g. fluoro, chloro or bromo, hydroxyl, C₁₋₄ hydroxylalkyl, e.g. hydroxymethyl or 2-hydroxyethyl, cyano, —CN, —NR¹⁶R¹⁷, e.g. methylamino or dimethylamino, —CH₂NR¹⁶R¹⁷, e.g. methylaminomethyl, —CH₂NR¹⁸C(O)R¹⁸, e.g. CH₂NHC(O)CH₃, CH₂NR¹⁶C(O)OR¹⁸, e.g. —CH₂NHC(O)₂CH₃, CH₂NR¹⁶C(O)NR¹⁶R¹⁷, e.g. —CH₂NHC(O)NHCH₃, CH₂NR¹⁶S(O)ₚR¹⁸, e.g. —CH₂NHS(O)₂CH₃, —S(O)₂NR¹⁶R¹⁷, e.g. —S(O)₂NHCH₃, heterocyclyl, e.g. piperidinyl, morpholinyl, piperazinyl, or heteroaryl, e.g. pyrimidine, pyrazole, pyrrole, thiophene, imidazole, tetrazole, triazole, pyridine, and pyrazine, and w is 1-3.

Another embodiment of the invention is where Y is

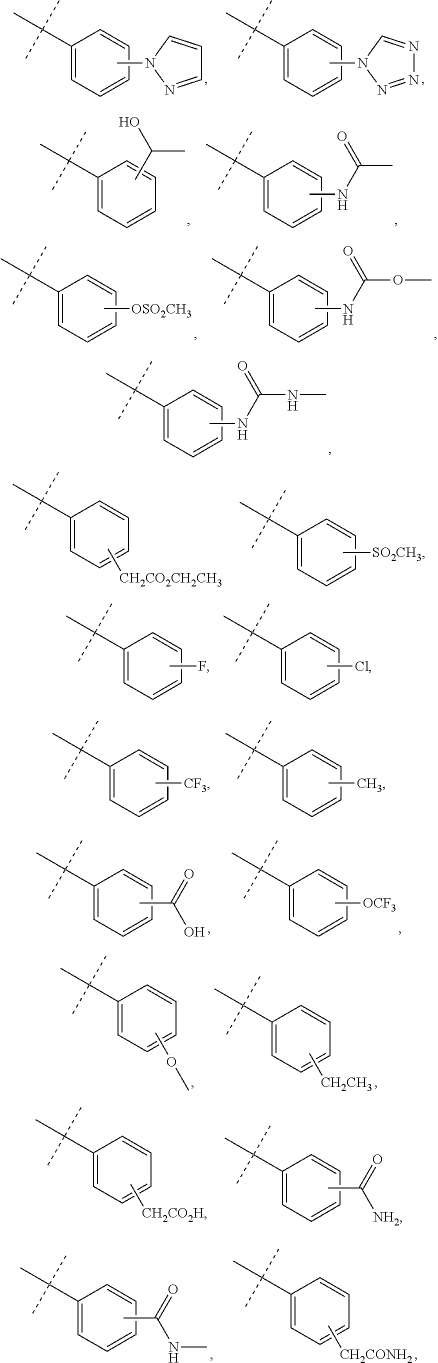

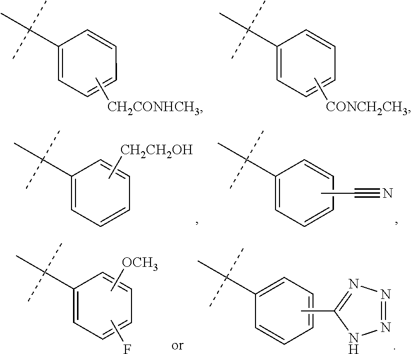

Another embodiment of the invention is where Y is

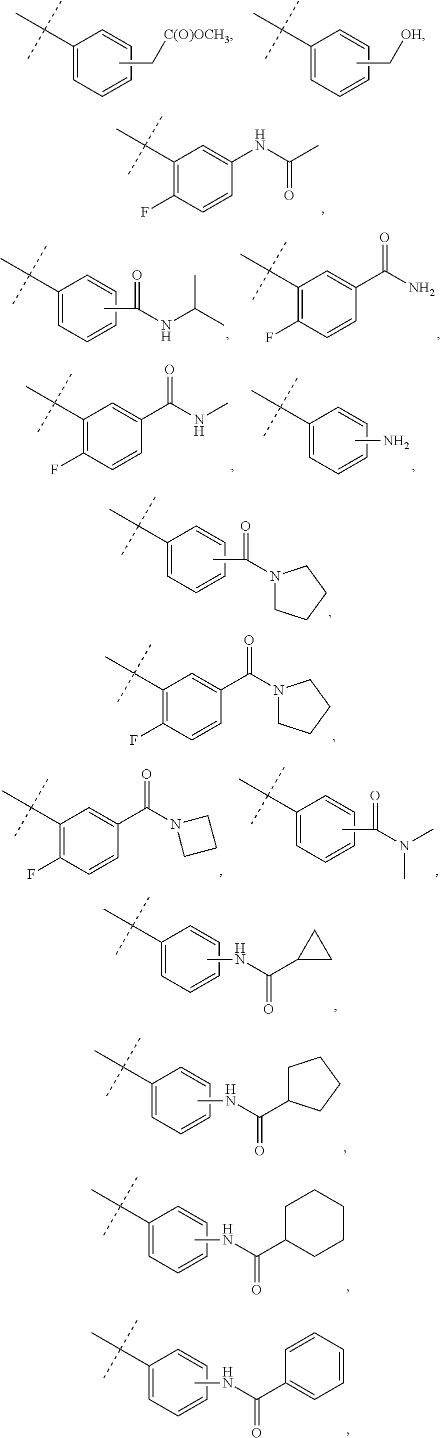

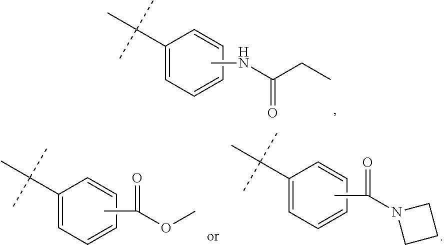

Another embodiment of the invention is where Y is

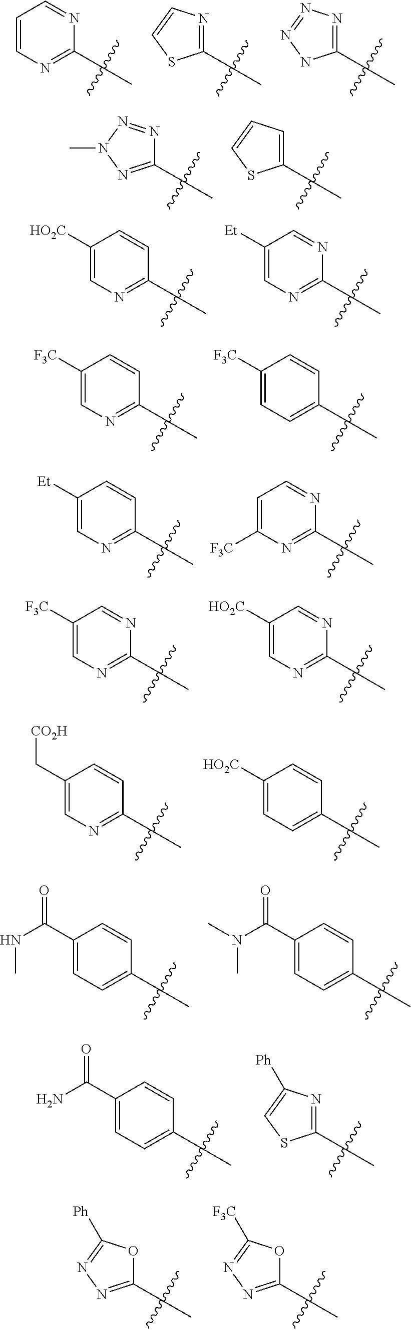

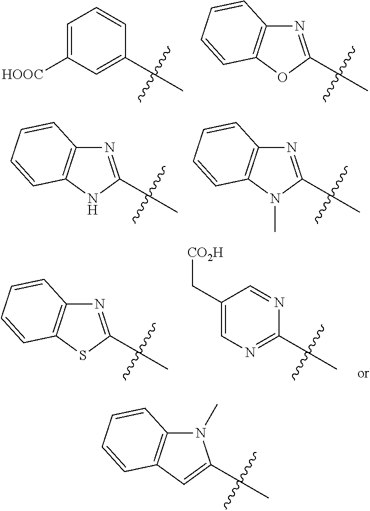

Another embodiment of the invention is where Y is

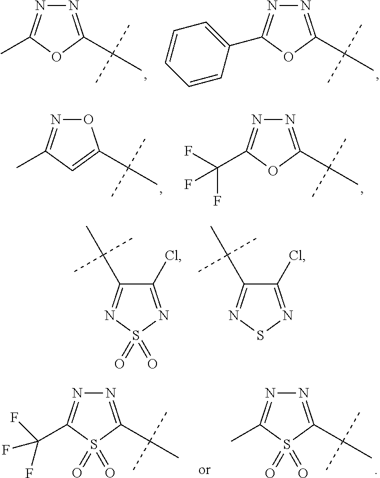

A specific embodiment of the compounds of formulae (I), (II), (III), (IV) or (V) is selected from:
(4-{(2S,3S,4R,5R,6S)-6-[4-Chloro-3-(4-ethoxybenzyl)-phenyl]-3,4,5-trihydroxy tetrahydropyran-2-ylmethylsulfanyl}phenyl)acetic acid methyl ester;
(4-{(2S,3S,4R,5R,6S)-6-[4-chloro-3-(4-ethoxybenzyl)phenyl]-3,4,5-trihydroxytetrahydro-pyran-2-ylmethylsulfanyl}phenyl)-acetic acid;
(4-{(2S,3S,4R,5R,6S)-6-[4-Chloro-3-(4-ethoxybenzyl)phenyl]-3,4,5-tri hydroxytetrahydropyran-2-ylmethylsulfanyl}phenyl)acetic acid carboxamide;
2-(4-{(2S,3S,4R,5R,6S)-6-[4-Chloro-3-(4-ethoxy-benzyl)-phenyl]-3,4,5-trihydroxy-tetrahydropyran-2-ylmethylsulfanyl}phenyl)-N-methylacetamide;
(2S,3R,4R,5S,6S)-2-[4-Chloro-3-(4-ethoxybenzyl)phenyl]-6-[4-(2-hydroxyethyl)-phenylsulfanylmethyl]tetrahydropyran-3,4,5-triol;
(4-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-phenyl)-acetic acid methyl ester;
(4-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-phenyl)-acetic acid;
(2S,3R,4R,5S,6S)-2-[4-Chloro-3-(4-ethoxybenzyl)phenyl]-6-(pyrimidin-2-ylsulfanylmethyl) tetrahydropyran-3,4,5-triol;
(2S,3R,4R,5S,6S)-2-[4-Chloro-3-(4-ethoxybenzyl)phenyl]-6-(thiazol-2-ylsulfanylmethyl)tetrahydropyran-3,4,5-triol;
(2S,3R,4R,5S,6S)-2-[4-Chloro-3-(4-ethoxybenzyl)phenyl]-6-(1-methyl-1H-tetrazol-5-ylsulfanylmethyl)tetrahydropyran-3,4,5-triol:
(2S,3R,4R,5S,6S)-2-[4-Chloro-3-(4-ethoxybenzyl)-phenyl]-6-(thiophen-2-ylsulfanylmethyl)tetrahydropyran-3,4,5-triol; and
(2S,3R,4R,5S,6S)-2-[4-Chloro-3-(4-ethoxybenzyl)phenyl]-6-(5-methyl-1,1-dioxo-1H-1lambda*6*-[1,3,4]thiadiazol-2-ylsulfanylmethyl)-tetrahydro-pyran-3,4,5-triol;
(2S,3R,4R,5S,6S)-2-[4-Chloro-3-(4-ethoxy-benzyl)-phenyl]-6-(4-phenyl-thiozol-2-ylsulfanylmethyl)-tetrahydro-pyran-3,4,5-triol;
(2S,3S,4R,5R,6S)-2-(Benzothiazol-2-ylsulfanylmethyl)-6-[4-chloro-3-(4-ethoxy-benzyl)-phenyl]-tetrahydro-pyran-3,4,5-triol;
(2S,3S,4R,5R,6S)-2-(1H-Benzoimidazol-2-ylsulfanyl methyl)-6-[4-chloro-3-(4-ethoxy-benzyl)-phenyl]-tetrahydro-pyran-3,4,5-triol;
(2S,3R,4R,5S,6S)-2-[4-Chloro-3-(4-ethoxy-benzyl)-phenyl]-6-(5-trifluoromethyl-pyridin-2-ylsulfanylmethyl)-tetrahydro-pyran-3,4,5-triol;
(2S,3R,4R,5S,6S)-2-[4-Chloro-3-(4-ethoxy-benzyl)-phenyl]-6-(pyrimidin-2-ylsulfonylmethyl)-tetrahydro-pyran-3,4,5-triol;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid ethyl ester;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid;
(2S,3R,4R,5S,6S)-2-[4-Chloro-3-(4-ethoxy-benzyl)-phenyl]-6-(4-hydroxymethyl-phenylsulfanylmethyl)-tetrahydro-pyran-3,4,5-triol;
6-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-nicotinic acid;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid;
(2S,3R,4R,5S,6S)-2-[4-Chloro-3-(4-ethoxy-benzyl)-phenyl]-6-(2-hydroxymethyl-phenylsulfanylmethyl)-tetrahydro-pyran-3,4,5-triol;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-acetamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-N-methyl-benzamide;
(4-{(2S,3S,4R,5R,6S)-6-[4-Chloro-3-(4-ethoxy-benzyl)-phenyl]-3,4,5-trihydroxy-tetrahydro-pyran-2-ylmethanesulfinyl}-phenyl)-acetic acid;
(2S,3S,4R,5R,6S)-2-(2-Amino-phenylsulfanylmethyl)-6-[4-chloro-3-(4-ethoxy-benzyl)-phenyl]-tetrahydro-pyran-3,4,5-triol;
(2S,3S,4R,5R,6S)-2-(3-Amino-phenylsulfanylmethyl)-6-[4-chloro-3-(4-ethoxy-benzyl)-phenyl]-tetrahydro-pyran-3,4,5-triol;
(2S,3S,4R,5R,6S)-2-(4-Amino-phenylsulfanylmethyl)-6-[4-chloro-3-(4-ethoxy-benzyl)-phenyl]-tetrahydro-pyran-3,4,5-triol;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid;
2-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
2-(3-{(2S,3S,4R,5R,6S)-6-[4-chloro-3-(4-ethoxy-benzyl)-phenyl]-3,4,5-trihydroxy-tetrahydro-pyran-2-ylmethanesulfonyl}-phenyl)-acetamide;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-pyrrolidin-1-yl-methanone;
Azetidin-1-yl-(3-{(2S,3S,4R,5R,63)-6-[4-chloro-3-(4-ethoxy-benzyl)-phenyl]-3,4,5-trihydroxy-tetrahydro-pyran-2-ylmethylsulfanyl}-4-fluoro-phenyl)-methanone;
2-{(2S,3S,4R,5R,6S)-6-[4-Chloro-3-(4-ethoxy-benzyl)-phenyl]-3,4,5 trihydroxytetrahydro-pyran-2 ylmethylsulfanyl}-N-methyl benzamide;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-ethyl-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-methyl-benzamide;
3-{(2S,3S,4R,5R,6S)-6-[4Chloro-3-(4-ethoxy-benzyl)-phenyl]-3,4,5-trihydroxy-tetrahydro-pyran-2-ylmethylsulfanyl}-N-ethyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-methyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide;
(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1 yl-methanone;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1 yl-methanone;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide;
(4-{(2S,3S,4R,5R,6S)-6-[4Chloro-3-(4-ethoxy-benzyl)-phenyl]-3,4,5-trihydroxy-tetrahydro-pyran-2-ylmethylsulfanyl}-phenyl)-pyrrolidin-1 yl-methanone;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide;
4-{(2S,3S,4R,5R,6S)-6-[4-Chloro-3-(4-ethoxy-benzyl)phenyl]-3,4,5-trihydroxy-tetrahydro-pyran-2-ylmethylsulfanyl}-N,N-dimethyl-benzamide;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
Cyclopentanecarboxylic acid (3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
Cyclohexanecarboxylic acid (3-{(2S,3S,4R,5R,6S)-6-[4-chloro-3-(4-ethoxy-benzyl)-phenyl]-3,4,5-trihydroxy-tetrahydro-pyran-2ylmethylsulfanyl}-phenyl)-amide;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
Cyclohexanecarboxylic acid (4-{(2S,3S,4R,5R,6S)-6-[4-chloro3-(4-ethoxy-benzyl)-phenyl]-3,4,5-trihydroxy-tetrahydro-pyran-2-ylmethylsulfanyl}-phenyl)-amide;
Cyclopentanecarboxylic acid (4-{(2S,3S,4R,5R,6S)-6-[4-chloro-3-(4-ethoxybenzyl)-phenyl]-3,4,5-trihydroxy-tetrahydro-pyran-2-ylmethylsulfanyl}-phenyl)-amide;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
Cyclohexanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopentanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopropanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopropanecarboxylic acid (3-{(2S,3,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamic acid methyl ester;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamic acid methyl ester;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamicacid methyl ester;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
   Cyclopropanecarboxylic acid (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
(2S,3R,4R,5S,6S)-2-[4-Chloro-3-(4-ethoxy-benzyl)-phenyl]-6-[4-(2-hydroxy ethyl)benzenesulfonylmethyl]-tetrahydro-pyran-3,4,5-triol;
(2S,3R,4R,5S,6S)-2-[4-Chloro-3-(4-ethoxy-benzyl)-phenyl]-6-[3-(2-hydroxy-ethyl)benzenesulfonylmethy]-tetrahydro-pyran-3,4,5-triol;
(2S,3R,4R,5S,6S)-2-[4-Chloro-3-(4-ethoxy-benzyl)-phenyl]-6-[3-(2-hydroxy-ethyl)phenylsulfanylmethyl]-tetrahydro-pyran-3,4,5-triol;
(2S,3R,4R,5S,6S)-2-[4-Chloro-3-(4-ethoxy-benzyl)-phenyl]-6-(3-hydroxymethyl-phenylsulfanylmethyl)-tetrahydro-pyran-3,4,5-triol;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-benzamide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
N-(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-acetamide;
3-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide;
3-{(2R,3S,4R,5R,6S)-6-[4-Chloro-3-(4-ethoxy-benzyl)-phenyl]-3,4,5-trihydroxy-tetrahydro-pyran-2-ylmethoxy}-N-methyl-benzamide;
(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-pyrrolidin-1-yl-methanone;
Azetidin-1-yl-(3-{(2R,3S,4R,5R,6S)-6-[4-chloro-3-(4-ethoxy-benzyl)-phenyl]-3,4,5-trihydroxy-tetrahydro-pyran-2-ylmethoxy}-phenyl)-methanone;
4-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide;
   and pharmaceutically acceptable salts thereof.

A specific embodiment of the compounds of formulae (I), (II), or (III) is selected from:
(4-{(2S,3S,4R,5R,6S)-6-[4-Chloro-3-(4-ethoxybenzyl)-phenyl]-3,4,5-trihydroxy tetrahydropyran-2-ylmethylsulfanyl}phenyl)acetic acid methyl ester;
(4-{(2S,3S,4R,5R,6S)-6-[4-chloro-3-(4-ethoxybenzyl)phenyl]-3,4,5-trihydroxytetrahydro-pyran-2-ylmethylsulfanyl}phenyl)-acetic acid;
(4-{(2S,3S,4R,5R,6S)-6-[4-Chloro-3-(4-ethoxybenzyl)phenyl]-3,4,5-tri hydroxytetrahydropyran-2-ylmethylsulfanyl}phenyl)acetic acid carboxamide;
2-(4-{(2S,3S,4R,5R,6S)-6-[4-Chloro-3-(4-ethoxy-benzyl)-phenyl]-3,4,5-trihydroxy-tetrahydropyran-2-ylmethylsulfanyl}phenyl)-N-methylacetamide;
(2S,3R,4R,5R,6S)-2-[4-Chloro-3-(4-ethoxybenzyl)phenyl]-6-[4-(2-hydroxyethyl)-phenylsulfanylmethyl]tetrahydropyran-3,4,5-triol;
(2S,3R,4R,5S,6S)-2-[4-Chloro-3-(4-ethoxybenzyl)phenyl]-6-(pyrimidin-2-ylsulfanylmethyl)tetrahydropyran-3,4,5-triol; (2S,3R,4R,5S,6S)-2-[4-Chloro-3-(4-ethoxybenzyl)phenyl]-6-(thiazol-2-ylsulfanylmethyl)tetrahydropyran-3,4,5-triol;
(2S,3R,4R,5S,6S)-2-[4-Chloro-3-(4-ethoxybenzyl)phenyl]-6-(1-methyl-1H-tetrazol-5-ylsulfanylmethyl)tetrahydropyran-3,4,5-triol:
(2S,3R,4R,5S,6S)-2-[4-Chloro-3-(4-ethoxybenzyl)-phenyl]-6-(thiophen-2-ylsulfanylmethyl)tetrahydropyran-3,4,5-triol; and
(2S,3R,4R,5S,6S)-2-[4-Chloro-3-(4-ethoxybenzyl)phenyl]-6-(5-methyl-1,1-dioxo-1H-1lambda*6*-[1,3,4]thiadiazol-2-ylsulfanylmethyl)-tetrahydro-pyran-3,4,5-triol;
(2S,3R,4R,5S,6S)-2-[4-Chloro-3-(4-ethoxy-benzyl)-phenyl]-6-(4-phenyl-thiozol-2-ylsulfanylmethyl)-tetrahydro-pyran-3,4,5-triol;
(2S,3S,4R,5R,6S)-2-(Benzothiazol-2-ylsulfanylmethyl)-6-[4-chloro-3-(4-ethoxy-benzyl)-phenyl]-tetrahydro-pyran-3,4,5-triol;
(2S,3S,4R,5R,6S)-2-(1H-Benzoimidazol-2-ylsulfanylmethyl)-6-[4-chloro-3-(4-ethoxy-benzyl)-phenyl]-tetrahydro-pyran-3,4,5-triol;
(2S,3R,4R,5S,6S)-2-[4-Chloro-3-(4-ethoxy-benzyl)-phenyl]-6-(5-trifluoromethyl-pyridin-2-ylsulfanylmethyl)-tetrahydro-pyran-3,4,5-triol;
(2S,3R,4R,5S,6S)-2-[4-Chloro-3-(4-ethoxy-benzyl)-phenyl]-6-(pyrimidin-2-ylsulfonylmethyl)-tetrahydro-pyran-3,4,5-triol;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid ethyl ester;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid;
(2S,3R,4R,5S,6S)-2-[4-Chloro-3-(4-ethoxy-benzyl)-phenyl]-6-(4-hydroxymethyl-phenylsulfanylmethyl)-tetrahydro-pyran-3,4,5-triol;
6-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-nicotinic acid;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid;
(2S,3R,4R,5S,6S)-2-[4-Chloro-3-(4-ethoxy-benzyl)-phenyl]-6-(2-hydroxymethyl-phenylsulfanylmethyl)-tetrahydro-pyran-3,4,5-trial;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-acetamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-N-methyl-benzamide;
(4-{(2S,3S,4R,5R,6S)-6-[4-Chloro-3-(4-ethoxy-benzyl)-phenyl]-3,4,5-trihydroxy-tetrahydro-pyran-2-ylmethanesulfinyl}-phenyl)-acetic acid;
(2S,3S,4R,5R,6S)-2-(2-Amino-phenylsulfanylmethyl)-6-[4-chloro-3-(4-ethoxy-benzyl)-phenyl]-tetrahydro-pyran-3,4,5-triol;
(2S,3S,4R,5R,6S)-2-(3-Amino-phenylsulfanylmethyl)-6-[4-chloro-3-(4-ethoxy-benzyl)-phenyl]-tetrahydro-pyran-3,4,5-triol;
(2S,3S,4R,5R,6S)-2-(4-Amino-phenylsulfanylmethyl)-6-[4-chloro-3-(4-ethoxy-benzyl)-phenyl]-tetrahydro-pyran-3,4,5-triol;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid;
2-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
2-(3-{(2S,3S,4R,5R,6S)-6-[4-chloro-3-(4-ethoxy-benzyl)-phenyl]-3,4,5-trihydroxy-tetrahydro-pyran-2-ylmethanesulfonyl}-phenyl)-acetamide;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-pyrrolidin-1-yl-methanone;
Azetidin-1-yl-(3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-phenyl)-methanone;
2-{(2S,3S,4R,5R,6S)-6-[4-Chloro-3-(4-ethoxy-benzyl)-phenyl]-3,4,5 trihydroxytetrahydro-pyran-2 ylmethylsulfanyl}-N-methyl benzamide;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-ethyl-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-methyl-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-ethyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-methyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide;
4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide;
(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1-yl-methanone;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1-yl-methanone;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide;
(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1-yl-methanone;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide;
4-{(2S,3S,4R,5R,6S)-6-[4-Chloro-3-(4-ethoxy-benzyl)phenyl]-3,4,5-trihydroxy-tetrahydro-pyran-2-ylmethylsulfanyl}-N,N-dimethyl-benzamide;
2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide;
4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
Cyclopentanecarboxylic acid (3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
Cyclohexanecarboxylic acid (3-{(2S,3S,4R,5R,6S)-6-[4-chloro-3-(4-ethoxy-benzyl)-phenyl]-3,4,5-trihydroxy-tetrahydro-pyran-2ylmethylsulfanyl}-phenyl)-amide;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide;
Cyclohexanecarboxylic acid (4-{(2S,3S,4R,5R,6S)-6-[4-chloro3-(4-ethoxy-benzyl)-phenyl]-3,4,5-trihydroxy-tetrahydro-pyran-2-ylmethylsulfanyl}-phenyl)-amide;
Cyclopentanecarboxylic acid (4-{(2S,3S,4R,5R,6S)-6-[4-chloro-3-(4-ethoxybenzyl)-phenyl]-3,4,5-trihydroxy-tetrahydro-pyran-2-ylmethylsulfanyl}-phenyl)-amide;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
Cyclohexanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopentanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopropanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
Cyclopropanecarboxylic acid (3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamic acid methyl ester;
(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamic acid methyl ester;
N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamicacid methyl ester;
N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide;
Cyclopropanecarboxylic acid (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide;
(2S,3R,4R,5S,6S)-2-[4-Chloro-3-(4-ethoxy-benzyl)-phenyl]-6-[4-(2-hydroxy ethyl)benzenesulfonylmethyl]-tetrahydro-pyran-3,4,5-triol;
(2S,3R,4R,5S,6S)-2-[4-Chloro-3-(4-ethoxy-benzyl)-phenyl]-6-[3-(2-hydroxy-ethyl)benzenesulfonylmethyl]-tetrahydro-pyran-3,4,5-triol;
(2S,3R,4R,5S,6S)-2-[4-Chloro-3-(4-ethoxy-benzyl)-phenyl]-6-[3-(2-hydroxy-ethyl)phenylsulfanylmethyl]-tetrahydro-pyran-3,4,5-triol;
(2S,3R,4R,5S,68)-2-[4-Chloro-3-(4-ethoxy-benzyl)-phenyl]-6-(3-hydroxymethyl-phenylsulfanylmethyl)-tetrahydro-pyran-3,4,5-triol;
3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide;
3-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-benzamide;
N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide;
   and pharmaceutically acceptable salts thereof.

A specific embodiment of the compounds of formulae (I), (IV) or (V) is selected from:
N-(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-acetamide;
3-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide;
3-{(2R,3S,4R,5R,6S)-6-[4-Chloro-3-(4-ethoxy-benzyl)-phenyl]-3,4,5-trihydroxy-tetrahydro-pyran-2-ylmethoxy}-N-methyl-benzamide;
(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-pyrrolidin-1-yl-methanone;
Azetidin-1-yl-(3-{(2R,3S,4R,5R,6S)-6-[4-chloro-3-(4-ethoxy-benzyl)-phenyl]-3,4,5-trihydroxy-tetrahydro-pyran-2-ylmethoxy}-phenyl)-methanone;
4-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide;
   and pharmaceutically acceptable salts thereof.

In still another embodiment of the present invention, the compound forms a pharmaceutically acceptable salt, selected from a group comprising acid addition salts and base addition salts.

In still another embodiment of the present invention, the compound is a stereoisomer or a tautomer.

The compounds of the present invention are useful as both prophylactic and therapeutic treatments for diseases or conditions related to the inhibition of SGLT-2 and SGLT-1.

Thus, as a further aspect, the invention relates to a method for treating a disease or condition related to the inhibition of SGLT-2, comprising administration of an effective therapeutic amount of a compound of formula (I) or a pharmaceutically acceptable salt thereof.

Compounds of formula (I) may be useful in the treatment of metabolic disorders, or conditions such as (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia.

Compounds of formula (I) may be also suitable for preventing beta-cell degeneration such as apoptosis or necrosis of pancreatic beta cells, for improving or restoring the functionality of pancreatic cells, increasing the number and size of pancreatic beta cells, for use as diuretics or antihypertensives and for the prevention and treatment of acute renal failure.

As a further aspect, the invention relates to a method for treating a disorder selected from type 1 and type 2 diabetes mellitus, complications of diabetes, comprising administration of an effective amount of a compound of formula (I) or a pharmaceutically acceptable salt thereof.

A compound of formula (I) of the present invention may be usefully combined with another pharmacologically active compound, or with two or more other pharmacologically active compounds, for use in therapy. For example, a compound of the formula (I), or a pharmaceutically acceptable salt thereof, as defined above, may be administered simultaneously, sequentially or separately in combination with one or more agents for the treatment of disorders previously listed.

Therapeutic agents which are suitable for such a combination include, for example, antidiabetic agents such as metformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. GI 262570) and antagonists, PPAR-gamma/alpha modulators (e.g. KRP 297), alpha-glucosidase inhibitors (e.g. acarbose, voglibose), DPPIV inhibitors (e.g. LAF237, MK-431), alpha2-antagonists, insulin and insulin analogues, GLP-1 and GLP-1 analogues (e.g. exendin-4) or amylin. The list also includes inhibitors of protein tyrosinephosphatase 1, substances that affect deregulated glucose production in the liver, such as e.g. inhibitors of glucose-6-phosphatase, orfructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents such as for example HMG-CoA-reductase inhibitors (e.g. simvastatin, atorvastatin), fibrates (e.g. bezafibrate, fenofibrate), nicotinic acid and the derivatives thereof, PPAR-alpha agonists, PPAR-delta agonists, ACAT inhibitors (e.g. avasimibe) or cholesterol absorption inhibitors such as, for example, ezetimibe, bile acid-binding substances such as, for example, cholestyramine, inhibitors of ileac bile acid transport, HDL-raising compounds such as CETP inhibitors or ABC1 regulators or active substances for treating obesity, such as sibutramine or tetrahydrolipostatin, dexfenfluramine, axokine, antagonists of the cannabinoidi receptor, MCH-1 receptor antagonists, MC4 receptor agonists, NPY5 or NPY2 antagonists or β3-agonists such as SB-418790 or AD-9677 and agonists of the 5HT2c receptor.

Moreover, combinations with drugs for influencing high blood pressure, chronic heart failure or atherosclerosis such as e.g. A-II antagonists or ACE inhibitors, ECE inhibitors, diuretics, β-blockers, Ca-antagonists, centrally acting antihypertensives, antagonists of the alpha-2-adrenergic receptor, inhibitors of neutral endopeptidase, thrombocyte aggregation inhibitors and others or combinations thereof are suitable. Examples of angiotensin II receptor antagonists are candesartan cilexetil, potassium losartan, eprosartan mesylate, valsartan, telmisartan, irbesartan, EXP-3174, L-158809, EXP-3312, olmesartan, medoxomil, tasosartan, KT-3-671, GA-01 13, RU-64276, EMD-90423, BR-9701, etc. Angiotensin II receptor antagonists are preferably used for the treatment or prevention of high blood pressure and complications of diabetes, often combined with a diuretic such as hydrochlorothiazide.

A combination with uric acid synthesis inhibitors or uricosurics is suitable for the treatment or prevention of gout.

A combination with GABA-receptor antagonists, Na-channel blockers, topiramat, protein-kinase C inhibitors, advanced glycation end product inhibitors or aldose reductase inhibitors may be used for the treatment or prevention of complications of diabetes.

Such combinations may offer significant advantages, including synergistic activity, in therapy.

The present invention is also in relation to a pharmaceutical composition comprising a compound of formula 1 or its prodrug and pharmaceutically acceptable excipients.

In still another embodiment of the present invention, the prodrug is selected from a group comprising, esters and hydrates.

The term pro-drug is also meant to include any covalently bonded carries which release the active compound of the invention in vivo when such prodrug is administered to a mammalian subject. Pro-drugs of a compound of the invention may be prepared by modifying functional groups present in the compound of the invention in such a way that the modifications are cleaved, either in routine manipulation or in vivo, to the parent compound of the invention.

In still another embodiment of the present invention, the excipients are selected from a group comprising, binders, anti-adherents, disintegrants, fillers, diluents, flavors, colors, glidants, lubricants, preservatives, sorbents and sweeteners or combination(s) thereof.

In still another embodiment of the present invention, the composition is formulated into various dosage forms selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups and elixirs.

Dosages of agents of the invention employed in practicing the present invention will of course vary depending, for example, on the particular condition to be treated, the effect desired and the mode of administration. In general, suitable daily dosages for oral administration are of the order of 0.1 to 10 mg/kg.

### Method of Preparation

The invention provides, in another aspect, a process for preparing a compound of formula (I). The schemes detailed below show general schemes for synthesizing compounds of formula (I).

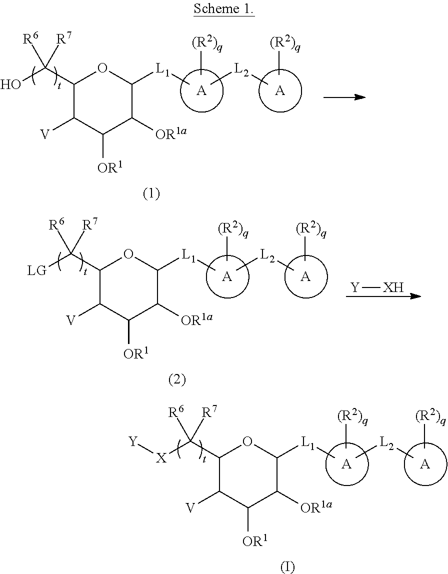

Compounds of formula 1 wherein m≠0 and all other symbols are as defined herein above may be converted to compounds of formula (2) wherein LG is a leaving group such as halide, tosyl or mesyl. The reaction may be carried by methods reported in the literature. Compounds of formula (2) may be reacted with Y—XH wherein X is S and Y is as defined herein above in the presence of a base and a solvent to provide compounds of formula (I) wherein m≠0, X is S and all other symbols are as defined herein above. When X═S, it may be further oxidized to sulfinyl or sulfonyl using suitable oxidation reagents and conditions well known in the literature. Also, functional groups as substitutents on Y may be transformed to different functional groups such as an ester function being converted to an acid, amide, hydroxymethyl, keto, aldehyde as well as an ester. The said conversions may be carried out using reagents and conditions well documented in the literature.

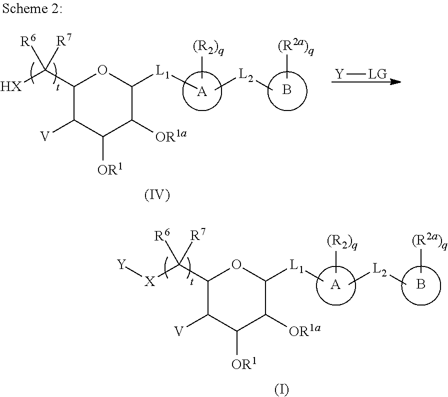

Compounds of formula IV wherein m≠0, X is S and all other symbols are as defined herein above may be reacted with Y-LG wherein LG is a leaving group such as halide, tosyl or mesyl. The reaction may be carried in the presence of a base and a solvent to provide compounds of formula (I) wherein m≠0, X is S and all other symbols are as defined herein above. When X═S, it may be further oxidized to sulfinyl or sulfonyl using suitable oxidation reagents and conditions well known in the literature. Also, functional groups as substitutents on Y may be transformed to different functional groups such as an ester function being converted to an acid, amide, hydroxymethyl, keto, aldehyde as well as an ester. The said conversions may be carried out using reagents and conditions well documented in the literature.

Compounds of formula (I) may be prepared from other compounds of formula (I) by methods well known to one skilled in the art.

It will be understood that the processes detailed above and elsewhere herein are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention. The term “about” in relation to a numerical value x means, for example, x±10%.

The following Examples are intended to illustrate the invention and are not to be construed as being limitations thereon. If not mentioned otherwise, all evaporations are performed under reduced pressure. The structure of final products, intermediates and starting materials is confirmed by standard analytical methods, e.g., microanalysis and spectroscopic characteristics, e.g. MS and NMR. Abbreviations used are those conventional in the art.

### EXAMPLE 1

### (4-{(2S,3S,4R,5R,6S)-6-[4-Chloro-3-(4-ethoxy-benzyl)-phenyl]-3,4,5-trihydroxy tetrahydro-pyran-2-ylmethylsulfanyl}-phenyl)-acetic acid methyl ester

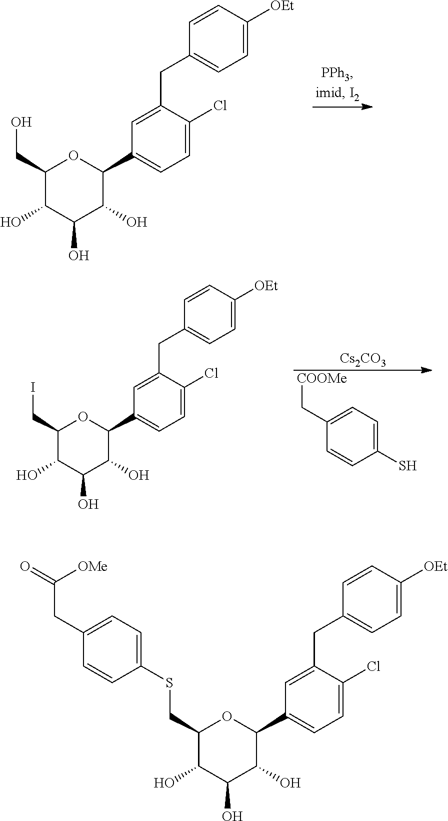

Step I. To a mixture of (2S,3R,4R,5S,6R)-2-[4-chloro-3-(4-ethoxy-benzyl)-phenyl]-6-hydroxymethyl-tetrahydro-pyran-3,4,5-triol (500 mg, 0.98 mmole) (prepared according to procedure as described in J. Med. Chem. 2008; 51 (5); 1145-1149), PPh₃ (450 mg, 1.6 mmole) and imidazole (101 mg, 1.5 mmole) in dichloromethane (DCM, 20 mL) was added iodine (400 mg, 1.5 mmole) at 0° C. and refluxed for 18 hrs. The reaction mixture was diluted with water (50 mL) and extracted with dichloromethane (2×200 mL). The crude product obtained after the removal of solvent was purified using silica gel column chromatography (0.5% methanol in DCM) to furnish 480 mg of (2S,3R,4R,5S,6S)-2-[4-chloro-3-(4-ethoxy-benzyl)-phenyl]-6-iodomethyl-tetrahydro-pyran-3,4,5-triol.

Step II. To a solution of above compound (400 mg, 0.74 mmole) in DMF (2 mL), cesium carbonate (660 mg, 1.6 mmole) was added followed by 4-mercaptophenylacetic acid methyl ester (176 mg, 0.89 mmole) at room temperature and stirred for 3-5 hours. The reaction mixture was diluted with water (50 mL) and extracted with DCM (2×200 mL). The crude product was purified by silica gel column chromatography (1% methanol in DCM) to furnish 350 mg the title compound.

¹H-NMR (400 M Hz, CDCl₃): δ 1.37 (t, J=7.2 Hz, 3H), 1.90 (s, 1H), 2.59 (s, 1H), 2.61 (s, 1H), 2.8 (s, 1H), 3.15 (m, 1H), 3.3-3.50 (m, 2H), 3.54 (s, 2H), 3.55-3.65 (m, 2H) 3.669 (s, 3H), 3.96-4.0 (m, 5H) 6.80 (d, J=8.4 Hz, 2H), 7.07 (m, 3H), 7.11 (d, J=1 Hz, 2H), 7.14 (s, 1H), 7.31-7.34 (m, 3H). MS (ES) m/z 595 (M+Na).

### EXAMPLE 2

### (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid

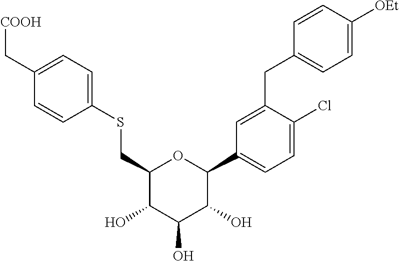

To a solution of (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid methyl ester (80 mg, 0.13 mmole) in a mixture of solvents (THF-MeOH—H₂O) (3:1:2, 5 mL) was added LiOH (15 mg, 0.26 mmole) and stirred for overnight at room temperature. The reaction mixture was concentrated; acidified with 1N HCl and extracted with DCM (2×50 mL). After the removal of solvent, the crude product was purified by HPLC to furnish title compound.

¹H-NMR (400 M Hz, CD₃OD): δ 1.37 (t, J=7.2 Hz, 3H), 3.0 (m, 1H), 3.2 (m, 1H), 3.3-3.51 (m, 6H), 3.94-4.04 (m, 5H), 6.80 (d, J=8.4 Hz, 2H), 7.01 (d, J=8.8 Hz, 2H), 7.1-7.15 (m, 3H), 7.21 (d, J=8.0 Hz, 1H), 7.30 (d, J=8.0 Hz, 3H). MS (ES) m/z 576 (M+18).

### EXAMPLE 3

### (4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid carboxamide

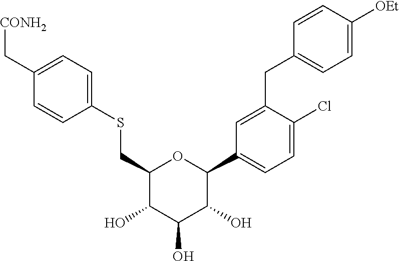

The compound (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid methyl ester (100 mg, 0.17 mmole) in methanolic ammonia (5.0 mL) was heated in sealed tube at 80° C. overnight. The reaction mixture was concentrated and purified by HPLC to furnish 30 mg the title compound.

¹H-NMR (400 M Hz, CD₃OD): δ 1.34 (t, J=7.2 Hz, 3H), 3.05-3.10 (m, 1H), 3.2 (m, 1H), 3.41-3.51 (m, 6H), 3.94-4.14 (m, 5H), 6.80 (d, J=8.4 Hz, 2H), 7.01 (d, J=8.8 Hz, 2H), 7.1-7.15 (m, 3H), 7.21 (d, J=8.0 Hz, 1H), 7.30 (m, 3H). MS (ES) m/z 558 (M+1).

### EXAMPLE 4

### 2-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-N-methyl-acetamide

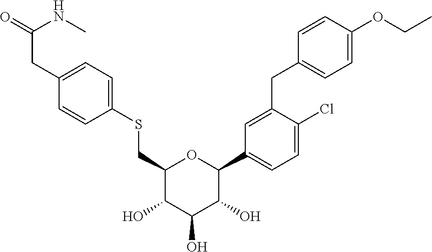

The compound (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid methyl ester (100 mg, 0.17 mmole) in 2M methanolic methylammonia (5.0 mL) solution was heated in sealed tube at 80° C. overnight. The reaction mixture was concentrated to furnish the title compound (80 mg).

¹H-NMR (400 M Hz, CD₃OD): δ 1.34 (t, J=7.2 Hz, 3H), 2.69 (s, 3H), 3.05-3.10 (m, 1H), 3.2 (m, 1H), 3.41-3.58 (m, 6H), 3.94-4.04 (m, 5H), 6.80 (d, J=8.4 Hz, 2H), 7.07 (d, J=8.4 Hz, 2H), 7.09 (d, J=2.0 Hz, 1H), 7.13 (d, J=8.0 Hz, 2H), 7.20 (d, J=2.0 Hz, 1H), 7.30 (d, J=8.0 Hz, 1H), 7.34 (d, J=8.0 Hz, 2H). MS (ES) m/z 558 (M+1).

### EXAMPLE 5

### (2S,3R,4R,5S,6S)-2-[4-Chloro-3-(4-ethoxy-benzyl)-phenyl]-6-[4-(2-hydroxy-ethyl)-phenylsulfanylmethyl]-tetrahydro-pyran-3,4,5-triol

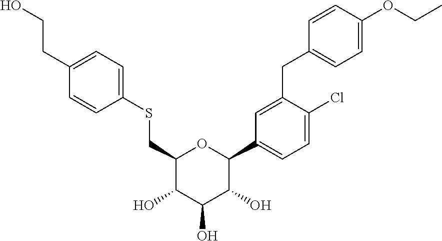

To a solution of (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid methyl ester (80 mg, 0.14 mmole) in THF-Water-MeOH mixture (1:1:1, 5 mL) was added NaBH₄ (10 mg, 0.27 mmole) and stirred for 6 h. On completion, the reaction mixture was diluted with water (10 mL), extracted with ethyl acetate (2×20 mL) and concentrated to get the crude product which was further purified by silica gel column chromatography (1% MeOH in DCM) to furnish the title compound (50 mg).

¹H-NMR (400 M Hz, CD₃OD): δ 1.39 (t, J=7.2 Hz, 3H), 2.72 (t, J=7.2 Hz, 2H), 3.05-3.07 (m, 1H), 3.23 (t, J=9.2 Hz, 2H), 3.39-3.42 (m, 3H), 3.68 (t, J=7.2 Hz, 2H), 3.94-34.03 (m, 5H), 6.79 (d, J=8.4 Hz, 2H), 7.056 (dd, J=1.6 & 8.0 Hz, 4H), 7.099 (dd, J=1.6 & 8.0 Hz, 1H), 7.198 (d, J=2.0 Hz, 1H), 7.279-7.31 (m, 3H). MS (ES) m/z 562 (M+18).

### EXAMPLE 6

### (4-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-phenyl)-acetic acid methyl ester

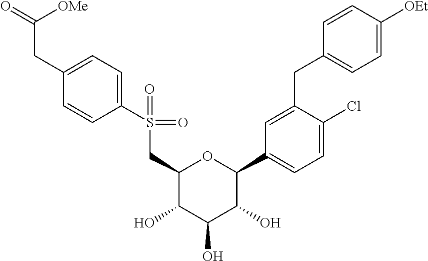

To a mixture of (4-{(2S,3S,4R,5R,6S)-6-[4-Chloro-3-(4-ethoxy-benzyl)-phenyl]-3,4,5-trihydroxy-tetrahydro-pyran-2-ylmethanesulfanyl}-phenyl)-acetic acid methyl ester (120 mg, 0.20 mmole) in DCM (10 mL) was added meta-chloroperbenzoic acid (m-CPBA) (72 mg, 0.41 mmole) at 0° C. and stirred for 3 h. at room temperature. The reaction mixture was diluted with water (10 mL) and extracted with dichloromethane (2×20 mL) and the organic layer washed with saturated solution of sodium bicarbonate (10 mL). The crude product obtained after the removal of solvent was purified by HPLC to furnish 120 mg of title compound.

¹H-NMR (400 M Hz, CDCl₃): δ 1.35 (t, J=7.2 Hz, 3H), 3.07-3.17 (m, 2H), 3.22-3.26 (m, 5H), 3.35-3.49 (m, 2H), 3.69 (s, 3H), 3.74 (t, J=9.2 Hz, 1H), 3.82 (d, J=9.6 Hz, 1H), 3.90 (d, J=13.2 Hz, 1H), 3.98 (q, J=6.8 Hz, 2H), 6.78 (dd, J=2.0, 8.0 Hz, 1H), 6.84 (d, J=8.8 Hz, 2H), 6.94 (d, J=8.4 Hz, 2H), 7.01 (d, J=1.6 Hz, 1H), 7.15-7.25 (d, J=8.8 Hz, 1H), 7.20 (d, J=8.4 Hz, 1H) 7.60 (d, J=8.0 Hz, 2H). MS (ES) m/z 605 (M+1).

### EXAMPLE 7

### (4-{(2S,3S,4R,5R,6S)-6-[4-Chloro-3-(4-ethoxy-benzyl)-phenyl]-3,4,6-trihydroxy-tetrahydro-pyran-2-ylmethanesulfonyl}-phenyl)-acetic acid

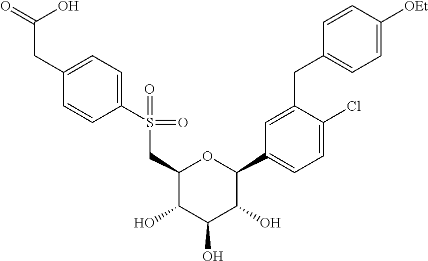

Title compound was prepared according to the procedure as described in example 2.

¹H-NMR (400 M Hz, CD₃OD): δ 1.35 (t, J=7.2 Hz, 3H), 3.14 (m, 2H), 3.30-3.50 (m, 4H), 3.62 (d, J=14.4 Hz, 1H), 3.73 (t, J=9.2 Hz, 1H), 3.83 (d, J=9.2 Hz, 1H), 3.90-4.08 (m, 4H), 6.80 (dd, J=1.6, 8.4 Hz, 1H), 6.84 (d, J=8.4 Hz, 2H), 6.95-7.02 (m, 3H), 7.16 (d, J=8.4 Hz, 2H), 7.22 (d, J=8.4 Hz, 1H), 7.59 (d, J=8.0 Hz, 2H). MS (ES) m/z 591 (M+1).

Following examples were obtained using analogous procedures described above.

### EXAMPLE 8

### (2S,3R,4R,5S,6S)-2-[4-Chloro-3-(4-ethoxy-benzyl)-phenyl]-6-(pyrimidin-2-ylsulfanylmethyl)-tetrahydro-pyran-3,4,5-triol

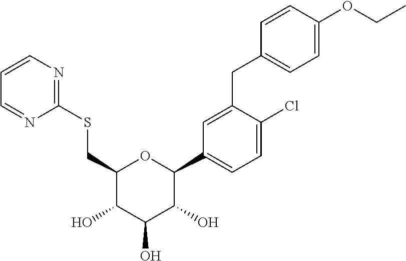

¹H-NMR (400 M Hz, CD₃OD): δ 1.35 (t, J=7.2 Hz, 3H), 3.22 (t, J=9.2 Hz, 2H), 3.30-3.45 (m, 3H), 3.61-3.62 (m, 1H), 3.85-3.95 (d, J=14.4 Hz, 1H), 3.94-3.99 (m, 3H), 4.09 (d, J=9.36 Hz, 1H), 6.81 (d, J=8.8 Hz, 2H), 7.05-7.11 (m, 2H), 7.14 (d, J=8.0 Hz, 1H), 7.20 (s, 1H), 7.30 (d, J=8.4 Hz, 2H), 8.52 (d, J=4.8 Hz, 2H). MS (ES) m/z 503 (M+1)

### EXAMPLE 9

### (2S,3R,4R,5S,6S)-2-[4-Chloro-3-(4-ethoxy-benzyl)-phenyl]-6-(thiazol-2-ylsulfanylmethyl)-tetrahydro-pyran-3,4,5-triol

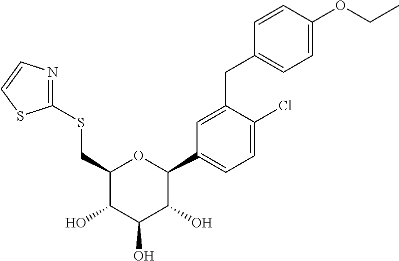

¹H-NMR (400 M Hz, CD₃OD): δ 1.35 (t, J=7.2 Hz, 3H), 3.18-3.23 (m, 1H), 3.29-3.50 (m, 3H), 3.61 (m, 2H), 3.94-4.079 (m, 4H), 4.07 (d, J=9.2 Hz, 1H), 6.81 (d, J=8.8 Hz, 2H), 7.05-7.11 (m, 3H), 7.15 (s, 1H), 7.30 (d, J=8.4 Hz, 1H), 7.35 (d, J=3.6 Hz, 1H), 7.58 (d, J=3.6 Hz, 1H). MS (ES) m/z 508 (M+1).

### EXAMPLE 10

### (2S,3R,4R,5S,6S)-2-[4-Chloro-3-(4-ethoxy-benzyl)-phenyl]-6-(1-methyl-1H-tetrazol-5-ylsulfanylmethyl)-tetrahydro-pyran-3,4,5-triol

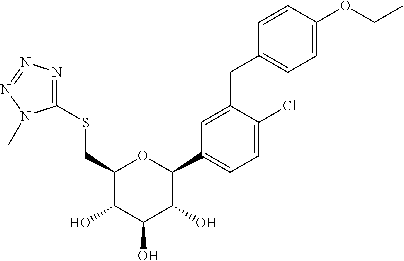

¹H-NMR (400 M Hz, CD₃OD): δ 1.35 (1, J=7.2 Hz, 3H), 3.196 (m, 1H), 3.30-3.50 (m, 3H), 3.57-3.60 (m, 1H), 3.73-3.788 (dd, J=2.4 & 14 Hz, 1H), 3.80 (s, 3H), 3.95-4.0 (m, 5H), 6.81 (d, J=8.8 Hz, 2H), 7.01 (dd, J=8.4 Hz, 1H), 7.02 (s, 1H), 7.09 (d, J=8.4 Hz, 2H), 7.29 (d, J=8.0 Hz, 1H). MS (ES) m/z 507 (M+1).

### EXAMPLE 11

### (2S,3R,4R,5S,6S)-2-[4-Chloro-3-(4-ethoxy-benzyl)-phenyl]-6-(thiophen-2-ylsulfanylmethyl)-tetrahydro-pyran-3,4,5-triol

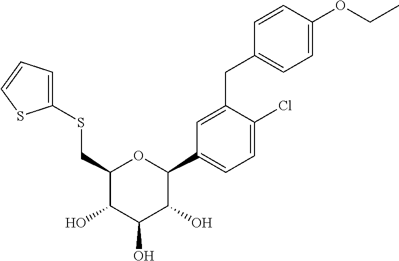

¹H-NMR (400 M Hz, CD₃OD): δ 1.34 (t, J=7.2 Hz, 3H), 2.96 (m, 2H), 3.13 (m, 1H), 3.30-3.47 (m, 3H), 3.96-4.03 (m, 5H), 6.79 (d, J=8.8 Hz, 2H), 6.78 (d, J=8.8 Hz, 2H), 6.87 (dd, J=5.6 & 4.0 Hz, 1H), 7.07-7.7.09 (m, 2H), 7.16 (dd, J=1.6 & 8.0 Hz, 2H), 7.33 (d, J=3.6 Hz, 1H). MS (ES) m/z 507 (M+1).

### EXAMPLE 12

### (2S,3R,4R,5S,6S)-2-[4-Chloro-3-(4-ethoxy-benzyl)-phenyl]-6-(5-methyl-1,1-dioxo-1H-1lambda*6*-[1,3,4]thiadiazol-2-ylsulfanylmethyl)-tetrahydro-pyran-3,4,5-triol

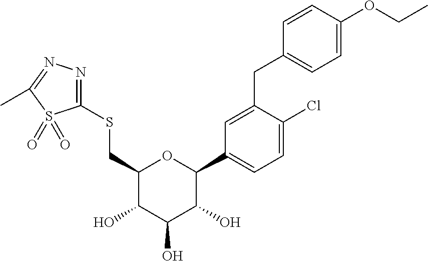

¹H-NMR (400 M Hz, CD₃OD): δ 1.35 (t, J=7.2 Hz, 3H), 2.58 (s, 3H), 3.23 (t, J=8.8 Hz, 1H), 3.3-3.47 (m, 3H), 3.64-3.68 (m, 2H), 3.9-4.07 (m, 5H), 6.81 (d, J=8.8 Hz, 2H), 7.03 (d, J=2.0 Hz, 1H), 7.06 (m, 2H), 7.12 (s, 1H), 7.31 (d, J=8.0 Hz, 1H). MS (ES) m/z 555 (M+1).

### EXAMPLE 13

### (2S,3R,4R,5S,6S)-2-[4-Chloro-3-(4-ethoxy-benzyl)-phenyl]-6-(4-phenyl-thiozol-2-ylsulfanylmethyl)-tetrahydro-pyran-3,4,6-triol

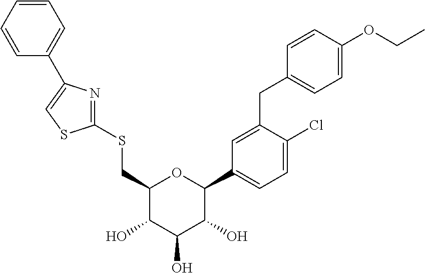

¹H-NMR (400 M Hz, CDCl₃): δ 1.40 (t, J=7.2 Hz, 3H), 2.18 (bs, 1H), 2.91 (bs, 1H), 3.43-3.50 (m, 2H), 3.71-3.77 (m, 3H), 3.98-4.08 (m, 5H), 4.21 (d, J=9.2 Hz, 1H), 6.08 (bs, 1H), 6.81 (d, J=8.8 Hz, 2H), 7.08 (d, J=8.8 Hz, 2H), 7.16-7.22 (m, 2H), 7.31-7.38 (m, 3H), 7.41-7.45 (m, 2H), 7.71-7.73 (m, J=7.2 Hz, 2H). MS (ES) m/z 584 (M+1).

### EXAMPLE 14

### (2S,3S,4R,5R,6S)-2-(Benzothiazol-2-ylsulfanylmethyl)-6-[4-chloro-3-(4-ethoxy-benzyl)-phenyl]-tetrahydro-pyran-3,4,5-triol

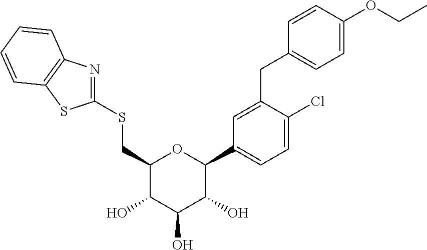

¹H-NMR (400 M Hz, CDCl₃): δ 1.40 (t, J=7.2 Hz, 3H), 2.18 (bs, 1H), 2.91 (bs, 1H), 3.48-3.51 (m, 2H), 3.60-3.64 (t, J=9.2 Hz, 1H), 3.72-3.80 (m, 2H), 3.98-4.14 (m, 5H), 4.21 (d, J=9.2 Hz, 1H), 6.25 (bs, 1H), 6.81 (d, J=8.8 Hz, 2H), 7.08 (d, J=8.8 Hz, 2H), 7.17-7.22 (m, 2H), 7.31-7.45 (m, 3H), 7.74-7.78 (m, 2H). MS (ES) m/z 558 (M+1).

### EXAMPLE 15

### (2S,3S,4R,5R,6S)-2-(1H-Benzoimidazol-2-ylsulfanylmethyl)-6-[4-chloro-3-(4-ethoxy-benzyl)-phenyl]-tetrahydro-pyran-3,4,5-triol

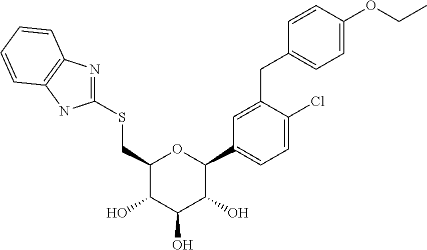

¹H-NMR (400 M Hz, CD₃OD): δ 1.35 (t, J=6.8 Hz, 3H), 3.22 (m, 1H), 3.45-3.49 (m, 2H), 3.61 (m, 1H), 3.67-3.72 (m, 3H), 3.86-3.99 (m, 3H), 4.05 (d, J=9.2 Hz, 1H), 6.76-6.79 (m, 3H), 6.92 (d, J=8 Hz, 1H), 6.96-7.00 (m, 3H), 7.26-7.33 (m, 4H). MS (ES) m/z 541 (M+1).

### EXAMPLE 16

### (2S,3R,4R,5S,6S)-2-[4-Chloro-3-(4-ethoxy-benzyl)-phenyl]-6-(5-trifluoromethyl-pyridin-2-ylsulfanylmethyl)-tetrahydro-pyran-3,4,5-triol

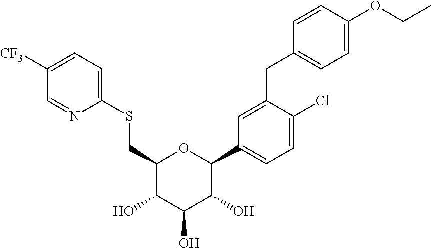

¹H-NMR (400 M Hz, CD₃OD): δ1.35 (t, J=7.2 Hz, 3H), 3.22 (t, J=9.2 Hz, 1H), 3.41-3.50 (m, 3H), 3.50-3.60 (m, 1H), 3.80-3.84 (m, 1H), 3.92-4.03 (m, 4H), 4.05 (d, J=9.6 Hz, 1H), 6.81 (d, J=8.4 Hz, 2H), 7.05-7.11 (m, 3H), 7.18 (bs, 1H), 7.26-7.29 (d, J=8.4 Hz, 1H), 7.46-7.48 (d, J=8.4 Hz, 1H), 7.77-7.79 (m, 1H), 8.65 (bs, 1H). MS (ES) m/z 570 (M+1).

### EXAMPLE 17

### (2S,3R,4R,5S,6S)-2-[4-Chloro-3-(4-ethoxy-benzyl)-phenyl]-6-(pyrimidin-2-ylsulfonylmethyl)-tetrahydro-pyran-3,4,5-triol

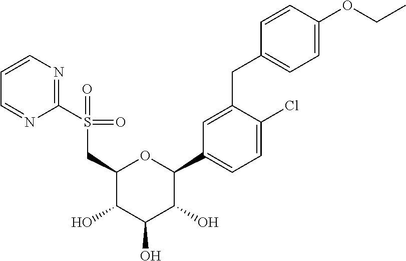

¹H-NMR (400 M Hz, CD₃OD): δ 1.36 (t, J=7.2 Hz, 3H), 3.11 (t, J=9.2 Hz, 1H), J=9.2 Hz, 1H), 3.35 (t, J=8.8 Hz, 1H), 3.64-3.79 (m, 3H), 3.90-4.06 (m, 5H), 6.70 (dd, J₁=2 Hz, J₂=8.4 Hz 1H), 6.85-6.91 (m, 4H), 7.19 (d, J=8.4 Hz, 2H), 7.25 (d, J=8.0 Hz, 1H), 8.32 (d, J=4.8 Hz, 2H). MS (ES) m/z 535 (M+1)

### EXAMPLE 18

### 4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid ethyl ester

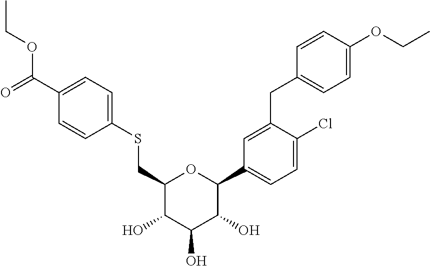

¹H-NMR (400 M Hz, CD₃OD): δ 1.37 (m, 6H), 3.17-3.21 (m, 2H), 3.42-3.47 (m, 2H), 3.55-3.59 (m, 2H), 3.90-4.04 (m, 5H), 4.31 (q, J=6.8 Hz, 2H), 6.76 (d, J=8.8 Hz, 2H), 7.01 (d, J=8.8 Hz, 3H), 7.11 (m, 1H), 7.22-7.24 (d, J=8.0 Hz, 1H), 7.41 (d, J=8.8 Hz, 2H), 7.79 (d, J=8.8 Hz, 2H). MS (ES) m/z 573 (M+1).

### EXAMPLE 19

### 4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid

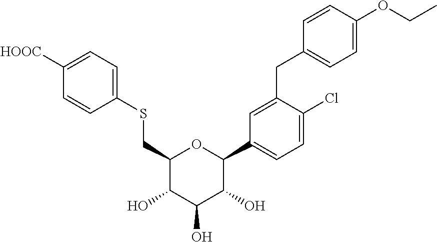

¹H-NMR (400 M Hz, CD₃OD): δ 1.35 (t, J=7.2 Hz, 3H), 3.18-3.22 (m, 2H), 3.42-3.47 (m, 2H), 3.55-3.59 (m, 2H), 3.88-4.05 (m, 5H), 6.76 (d, J=8.8 Hz, 2H), 7.03-7.06 (m, 3H), 7.13 (m, 1H), 7.25-7.27 (d, J=8.4 Hz, 1H), 7.41-7.43 (d, J=8.4 Hz, 2H), 7.82-7.84 (d, J=8.4 Hz, 2H). MS (ES) m/z 545 (M+1).

### EXAMPLE 20

### (2S,3R,4R,5S,6S)-2-[4-Chloro-3-(4-ethoxy-benzyl)-phenyl]-6-(4-hydroxymethyl-phenylsulfanylmethyl)-tetrahydro-pyran-3,4,5-triol

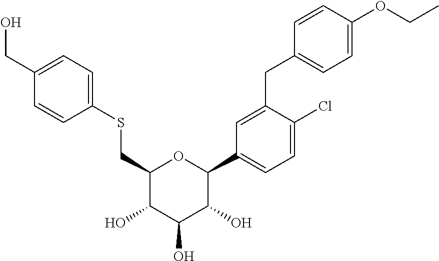

¹H-NMR (400 M Hz, CD₃OD): δ 1.36 (t, J=7.2 Hz, 3H), 3.1 (dd, J=7.2 Hz, 6.8 Hz, 1H), 3.21 (m, 1H), 3.40-3.55 (m, 4H), 3.92-4.04 (m, 5H), 4.51 (s, 2H), 6.78 (d, J=8.4 Hz, 2H), 7.06-7.11 (m, 3H), 7.18-7.20 (m, 3H), 7.28-7.30 (d, J=8.0 Hz, 1H), 7.34-7.37 (d, J=8.4 Hz, 2H). MS (ES) m/z 548 (M+18).

### EXAMPLE 21

### 6-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-nicotinic acid

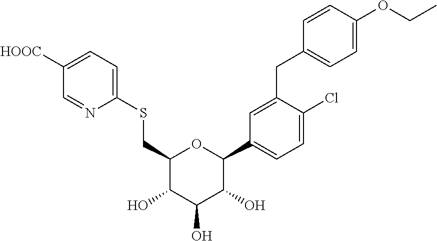

¹H-NMR (400 M Hz, CD₃OD): δ 1.35 (t, J=7.2 Hz, 3H), 3.22 (t, J=9.2 Hz, 1H), 3.41-3.53 (m, 3H), 3.62 (m, 1H), 3.74-3.78 (m, 1H), 3.92-4.03 (m, 4H), 4.05 (d, J=9.6 Hz, 1H), 6.88 (d, J=8.8 Hz, 2H), 7.04-7.11 (m, 3H), 7.16 (bs, 1H), 7.26-7.28 (d, J=8.4 Hz, 1H), 7.40-7.42 (d, J=8.4 Hz, 1H), 8.03-8.06 (m, 1H), 8.90 (bs, 1H).

MS (ES) m/z 546 (M+1)

### EXAMPLE 22

### 2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzoic acid

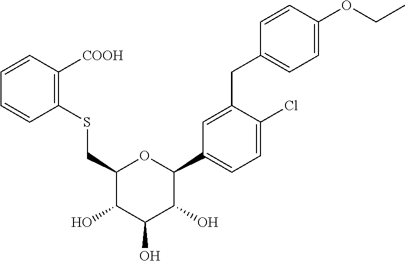

¹H-NMR (400 M Hz, CD₃OD): δ 1.34 (t, J=7.2 Hz, 3H), 3.05-3.11 (dd, J=7.6 Hz, 6.4 Hz, 1H), 3.23 (t, J=9.2 Hz, 1H), 3.40-3.52 (m, 3H), 3.58-3.60 (m, 1H), 3.91-3.98 (m, 4H), 4.05 (d, J=9.6 Hz, 1H), 6.76 (d, J=8.8 Hz, 2H), 7.03-7.05 (d, J=8.8 Hz, 2H), 7.09-7.15 (m, 2H), 7.20 (bs, 1H), 7.26-7.28 (d, J=8.4 Hz, 1H), 7.32-7.36 (m, 1H), 7.54-7.56 (d, J=8.4 Hz, 1H), 7.87-7.89 (m, 1H). MS (ES) m/z 545 (M+1).

### EXAMPLE 23

### (2S,3R,4R,5S,6S)-2-[4-Chloro-3-(4-ethoxy-benzyl)-phenyl]-6-(2-hydroxymethyl-phenylsulfanylmethyl)-tetrahydro-pyran-3,4,5-triol

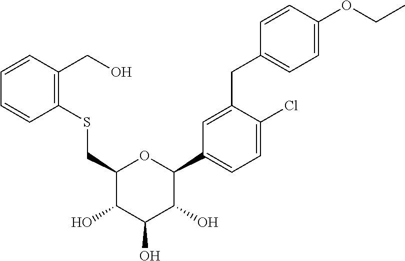

¹H-NMR (400 M Hz, CD₃OD): δ 1.34 (t, J=7.2 Hz, 3H), 3.03-3.13 (m, 1H), 3.26 (t, J=9.2 Hz, 1H), 3.39-3.54 (m, 4H), 3.73-3.77 (m, 1H), 3.85-3.88 (m, 1H), 3.94-4.02 (m, 4H), 4.09 (d, J=9.6 Hz, 1H), 6.80 (d, J=8.8 Hz, 2H), 7.06-7.10 (m, 3H), 7.14-7.18 (m, 1H), 7.22-7.25 (m, 2H), 7.28-7.38 (m, 2H), 7.47-7.50 (m, 1H).

MS (ES) m/z 548 (M+18).

### EXAMPLE 24

### 2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide

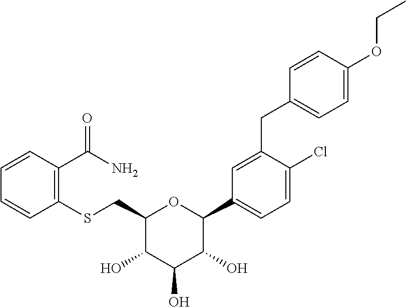

¹H-NMR (400 M Hz, CD₃OD): δ 1.34 (t, J=7.2 Hz, 3H), 3.09-3.14 (dd, J=7.2 Hz, 6.4 Hz, 1H), 3.23 (t, J=9.2 Hz, 1H), 3.41-3.56 (m, 4H), 3.95-4.05 (m, 5H), 6.78 (d, J=8.8 Hz, 2H), 7.06-7.08 (d, J=8.8 Hz, 2H), 7.11-7.20 (m, 3H), 7.25-7.31 (m, 2H), 7.39-7.41 (m, 1H), 7.56-7.58 (d, J=8.0 Hz, 1H). MS (ES) m/z 544 (M+1).

### EXAMPLE 25

### N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-acetamide

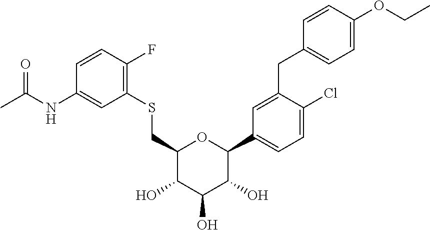

Step I. Thionyl chloride (12 mL) was added to water (72 mL) under ice cooling and stirred at room temperature for 18 hrs. Copper(I) chloride (42 mg) was added at −3 to 0° C. and resulting yellowish green solution was stored at −3° C.

Step II. To a mixture of 2-fluoro-5-nitroaniline (2.0 g, 12.8 mmole) in conc. HCl (15 mL) NaNO₂ solution (1.05 g, 15.3 mmole, in 1.5 mL water) added at −10° C. and stirred for 1 hour to get yellow diazonium salt.

Step III. Above Diazonium salt obtained in step II was added dropwise to the solution obtained in step I at −10 to 0° C. and stirred for 1 hour at 0° C. and 30 min at room temperature. The reaction mixture was diluted with water (250 mL) and extracted with diethyl ether (3×25 mL), resulting organic layer washed with saturated sodium bicarbonate (25×2 mL). Removal of solvent furnished 2-fluoro-5-nitro-benzenesulfonyl chloride (1.4 g).

Step IV. To a mixture of 2-fluoro-5-nitro-benzenesulfonyl chloride (1.4 g, 5.85 mmole) in conc. HCl (7 mL), SnCl₂.2H₂O (6.6 g, 29.2 mmole) was added and reaction mixture heated to 100° C. The reaction mixture was cooled to room temperature and conc. HCl (3 mL) was added and filtered through celite bed, filtrate was basified with saturated sodium bicarbonate and extracted with dichloromethane (2×25 mL). Removal of solvent furnished crude 2-fluoro-5-amino-benzenethiol (0.48 g) which was used as such for the next step.

Step V. To an ice cold solution of (2S,3R,4R,5S,6S)-2-[4-chloro-3-(4-ethoxy-benzyl)-phenyl]-6-iodomethyl-tetrahydro-pyran-3,4,5-triol (150 mg, 0.28 mmole) in DMF (2 mL), cesium carbonate (182 mg, 0.56 mmole) was added followed by crude 2-fluoro-5-amino-benzenethiol (200 mg, 1.12 mmole) at room temperature and stirred for 5 hrs. The reaction mixture was diluted with water (50 mL) and extracted with dichloromethane (2×20 mL). The crude product was purified by silica gel column chromatography (1% methanol in dichloromethane) to furnish (2S,3S,4R,5R,6S)-2-(5-amino-2-fluoro-phenylsulfanylmethyl)-6-[4-chloro-3-(4-ethoxy-benzyl)-phenyl]-tetrahydro-pyran-3,4,5-triol (85 mg).

Step VI. To a solution of (2S,3S,4R,5R,6S)-2-(5-amino-2-fluoro-phenylsulfanylmethyl)-6-[4-chloro-3-(4-ethoxy-benzyl)-phenyl]-tetrahydro-pyran-3,4,5-triol (60 mg, 0.11 mmole) in pyridine (0.5 mL), acetic anhydride (0.3 mL), DMAP (1 mg) was added at room temperature and stirred overnight. The reaction mixture was diluted with water (10 mL) and acidified with 1N HCl, extracted with dichloromethane (2×20 mL). The product obtained after the removal of solvent furnished acetic acid (2S,3S,4R,5S,6S)-4,5-diacetoxy-2-(5-amino-2-fluoro-phenylsulfanylmethyl)-6-[4-chloro-3-(4-ethoxy-benzyl)-phenyl]-tetrahydro-pyran-3-yl ester (85 mg).

Step VII. To a solution of acetic acid (2S,3S,4R,5S,6S)-4,5-diacetoxy-2-(5-amino-2-fluoro-phenylsulfanylmethyl)-6-[4-chloro-3-(4-ethoxy-benzyl)-phenyl]-tetrahydro-pyran-3-yl ester (85 mg, 0.12 mmole) in THF:MeOH:H₂O (3:1:2, 13 mL), lithium hydroxide (10 mg, 0.24 mmole) was added room temperature and stirred overnight. The reaction mixture was diluted with water (10 mL) and extracted with dichloromethane (3×20 mL). The crude product obtained after the removal of solvent was purified using preparative HPLC to furnish N-(3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-phenyl)-acetamide (20 mg).

¹H-NMR (400 MHz, CD₃OD): δ 1.37 (t, J=7.2 Hz, 3H), 2.06 (s, 3H), 3.11-3.22 (m, 2H), 3.41-3.52 (m, 3H), 3.59 (t, J=6.8 Hz, 1H), 3.91-4.05 (m, 5H), 6.81 (d, J=8.8 Hz, 2H), 6.92 (t, J=9.6 Hz, 1H), 7.01 (d, J=8.4 Hz, 1H), 7.07 (d, J=8.4 Hz, 2H), 7.12 (s, 1H), 7.27 (d, J=8.0 Hz, 1H), 7.35-7.38 (m, 1H), 7.70 (dd, J=2.0 & 6.4 Hz, 1H).

MS (ES) m/z 576.1 (M+H).

### EXAMPLE 26

### 3-{(2S,3S,4R,5R,6S)-6-[4-Chloro-3-(4-ethoxy-benzyl)-phenyl]-3,4,6-trihydroxy-tetrahydro-pyran-2-ylmethylsulfanyl}-4-fluoro-benzamide

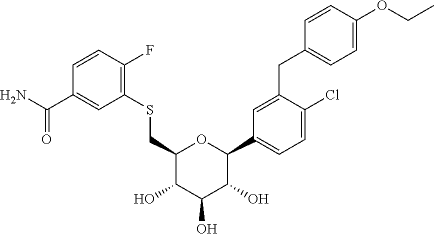

Step I. 4-Fluorobenzoic acid (3.0 g, 21.4 mmole) was added to ice cold solution of chlorosulfonic acid (9.0 mL). Reaction mixture was stirred at 130-140° C. for 8 hours. Reaction mixture added slowly to ice water (250 mL) and extracted with dichloromethane (3×25 mL). The crude product obtained after the removal of solvent furnished 3-chlorosulfonyl-4-fluoro-benzoic acid (3.3 g).

Step II. To a mixture of 3-chlorosulfonyl-4-fluoro-benzoic acid (3.2 g, 1.34 mmole) in conc. HCl (10 mL), SnCl₂.2H₂O (9.0 g, 4.0 mmole) was added and reaction mixture heated to 100° C. The reaction mixture was diluted with water (50 mL) and basified with saturated sodium bicarbonate, inorganic material was filtered and filtrate was acidified with 1N HCl and extracted with dichloromethane (4×25 mL). Solvent removal furnished 4-fluoro-3-mercapto-benzoic acid (2.5 g).

Step III. To a mixture of 4-fluoro-3-mercapto-benzoic acid (2.4 g, 13.9 mmole) in methanol, thionyl chloride (3.0 mL) was added at 0° C. and reaction mixture heated to 70° C. The reaction mixture was diluted with water (50 mL) extracted with ethyl acetate (3×25 mL). Solvent removal furnished crude 4-fluoro-3-mercapto-benzoic acid methyl ester (2.0 g).

Step IV. To an ice cold solution of (2S,3R,4R,5S,6S)-2-[4-chloro-3-(4-ethoxy-benzyl)-phenyl]-6-iodomethyl-tetrahydro-pyran-3,4,5-triol (100 mg, 0.19 mmole) in DMF (2 mL), cesium carbonate (125 mg, 0.38 mmole) was added followed by of 4-fluoro-3-mercapto-benzoic acid methyl ester (53 mg, 0.28 mmole) at room temperature and stirred for 5 hours. The reaction mixture was diluted with water (50 mL) and extracted with dichloromethane, (2×20 mL). The crude product was purified by silica gel column chromatography (1% methanol in dichloromethane) to furnish 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzoic acid methyl ester (80 mg).

Step IV. To a solution of 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzoic acid methyl ester (0.3 g, 0.52 mmole) in a mixture of solvents (THF-MeOH—H₂O) (3:1:2, 6 mL) was added LiOH (43 mg, 1.0 mmole) and stirred overnight at room temperature. The reaction mixture was concentrated; acidified with 1N HCl and extracted with dichloromethane (2×50 mL). Solvent removal furnished 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzoic acid (210 mg).

Step V. To a solution of 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzoic acid (100 mg, 0.17 mmole) in DMF (1 mL), HOBt (28 mg, 0.21 mmole) was added and stirred for 10 min., EDCl (40 mg, 0.21 mmole) was added and stirred at room temperature overnight. The reaction mixture was diluted with water (10 mL) and extracted with ethyl acetate (20 mL×2). The crude product obtained after the removal of solvent was purified using preparative HPLC to furnish 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzamide (30 mg).

¹H-NMR (400 MHz, CD₃OD): δ 1.38 (t, J=8.4 Hz, 3H), 3.21 (t, J=9.2 Hz, 2H), 3.41-3.52 (m, 3H), 3.62 (t, J=7.6 Hz, 1H), 3.93-4.06 (m, 5H), 6.82 (d, J=8.4 Hz, 2H), 7.00 (t, J=8.4 Hz, 2H), 7.10 (d, J=8.8 Hz, 3H), 7.28 (d, J=8.4 Hz, 1H), 7.62-7.7.68 (m, 1H), 8.06 (d, J=6.8 Hz, 1H). MS (ES) m/z 562.0 (M+H).

### EXAMPLE 27

### 3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-4-fluoro-N-methyl-benzamide

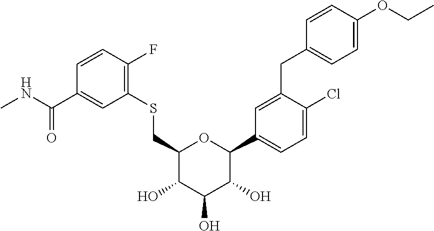

To a solution of 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-benzoic acid methyl ester (80 mg, 0.13 mmole) in 2M methanolic methylammonia (5.0 mL), 1,5,7 triazo bicycle[4,4,0] dec-5-ene (19 mg, 0.13) was added and heated in sealed tube at 90° C. for 72 hours. The reaction mixture was concentrated and purified by preparative HPLC to furnish 3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-N-methyl-benzamide (8.0 mg).

¹H-NMR (400 MHz, CD₃OD): δ 1.38 (t, J=8.4 Hz, 3H), 2.86 (s, 3H), 3.20 (dd, J=6.0 & 14.4 Hz, 2H), 3.41-3.52 (m, 3H), 3.60 (t, J=7.2 Hz, 1H), 3.90-4.04 (m, 5H), 6.80 (d, J=8.4 Hz, 2H), 6.97-7.00 (m, 2H), 7.05 (d, J=3.6 Hz, 1H), 7.07 (d, J=3.6 Hz, 2H), 7.25 (d, J=8.4 Hz, 1H), 7.59-7.62 (m, 1H), 7.99 (dd, J=1.6 & 6.8 Hz, 1H).

MS (ES) m/z 576.0 (M+H).

### EXAMPLE 28

### (4-{(2S,3S,4R,5R,6S)-6-[4-Chloro-3-(4-ethoxy-benzyl)-phenyl]-3,4,5-tri hydroxy-tetrahydro-pyran-2-ylmethanesulfinyl}-phenyl)-acetic acid

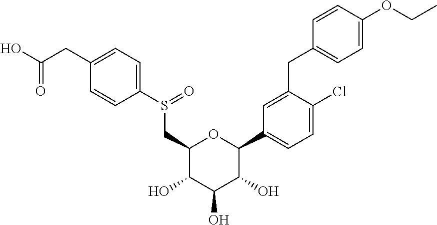

Step I. A solution of (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-acetic acid methyl ester (100 mg, 0.17 mmole) in hexafluoroisopropanol (1 mL), 50% aqueous H₂O₂ (20 μL, 0.34 mmole) at 0° C. and stirred for 2 hours. The reaction mixture was diluted with water (10 mL) and washed with aqueous sodium metabisulfide (2 mL), extracted with ethyl acetate (2×20 mL), removal of solvent furnished crude (4-(2S,3S,4R,5R,6S)-6-[4-chloro-3-(4-ethoxy-benzyl)-phenyl]-3,4,5-trihydroxy-tetrahydro-pyran-2-ylmethanesulfinyl}-phenyl)-acetic acid methyl ester (100 mg).

Step II. To a solution of (4-(2S,3S,4R,5R,6S)-6-[4-chloro-3-(4-ethoxy-benzyl)-phenyl]-3,4,5-trihydroxy-tetrahydro-pyran-2-ylmethanesulfinyl}-phenyl)-acetic acid methyl ester (100 mg, 0.17 mmole) in THF:MeOH:H₂O (3 mL), lithium hydroxide (15 mg, 0.34 mmole) was added at room temperature and stirred overnight. The reaction mixture was diluted with water (10 mL) and extracted with ethyl acetate (3×20 mL). The crude product obtained after the removal of solvent was purified using preparative HPLC to furnish (4-{(2S,3S,4R,5R,6S)-6-[4-Chloro-3-(4-ethoxy-benzyl)-phenyl]-3,4,5-trihydroxy-tetrahydro-pyran-2-ylmethanesulfinyl}-phenyl)-acetic acid (90 mg).

¹H-NMR (400 MHz, CD₃OD): δ 1.38 (t, J=8.4 Hz, 3H), 2.96-3.02 (m, 1H), 3.24-3.31 (m, 1H), 3.39 (t, J=6.8 Hz, 1H), 3.50 (t, J=8.8 Hz, 1H), 3.55 (s, 1H), 3.68 (s, 1H), 3.84 (d, J=9.2 Hz, 1H), 3.97-4.12 (m, 5H), 4.18 (d, J=9.6 Hz, 1H), 6.85 (t, J=8.0 Hz, 2H), 7.12-7.18 (m, 3H), 7.27 (d, J=7.6 Hz, 1H), 7.31 (d, J=8.0 Hz, 1H), 7.37 (t, J=8.4 Hz, 1H), 7.47 (d, J=8.0 Hz, 1H), 7.51 (d, J=8.0 Hz, 1H), 7.64 (d, J=8.0 Hz, 1H).MS (ES) m/z 575.2 (M+H).

### EXAMPLE 29

### (2S,3S,4R,5R,6S)-2-(2-Amino-phenylsulfanylmethyl)-6-[4-chloro-3-(4-ethoxy-benzyl)-phenyl]-tetrahydro-pyran-3,4,5-triol

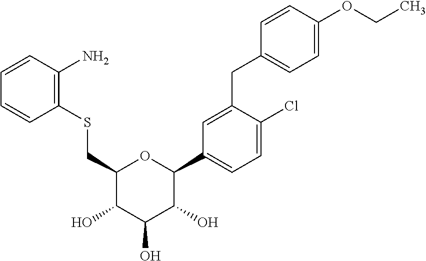

Title compound was prepared according to the procedure as described in example 1.

¹H-NMR (400 MHz, CD₃OD): δ 1.38 (t, J=7.0 Hz, 3H), 3.19-3.25 (m, 2H), 3.34-3.50 (m, 2H), 3.53-3.64 (m, 2H), 3.96-4.09 (m, 5H), 6.80 (d, J=8.0 Hz, 2H), 7.07 (d, J=8.0 Hz, 3H), 7.18 (d, J=1.2 Hz, 1H), 7.30 (d, J=8.0 Hz, 1H), 7.45 (d, J=8.0 Hz, 2H), 7.75 (d, J=8.0 Hz, 2H).

MS (ES) m/z 516.1 (M+H).

### EXAMPLE 30

### (2S,3S,4R,5R,6S)-2-(3-Amino-phenylsulfanylmethyl)-6-[4-chloro-3-(4-ethoxy-benzyl)-phenyl]-tetrahydro-pyran-3,4,5-triol

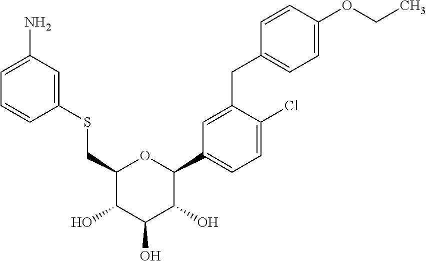

Title compound was prepared according to the procedure as described in example 1.

¹H-NMR (400 MHz, DMSO-d₆): δ 1.37 (t, J=7.2 Hz, 3H), 2.92-2.98 (m, 1H), 3.09-3.13 (m, 1H), 3.21-3.30 (m, 3H), 3.5 (m, 2H), 3.94-4.03 (m, 4H), 4.95 (d, J=6.0 Hz, 1H), 5.08-5.10 (m, 3H), 5.29 (d, J=5.2 Hz, 1H), 6.35 (d, J=7.6 Hz, 1H), 6.45 (d, J=7.6 Hz, 1H), 6.52 (s, 1H), 6.83 (d, J=8.4 Hz, 2H), 6.91 (t, J=8.0 Hz, 1H), 7.10 (d, J=8.4 Hz, 2H), 7.17 (d, J=8.4 Hz, 1H), 7.27 (s, 1H), 7.36 (d, J=8.4 Hz, 1H).

MS (ES) m/z 516.0 (M+H).

### EXAMPLE 31

### (2S,3S,4R,5R,6S)-2-(4-Amino-phenylsulfanylmethyl)-6-[4-chloro-3-(4-ethoxy-benzyl)-phenyl]-tetrahydro-pyran-3,4,5-triol

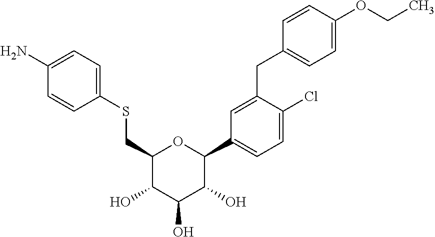

Title compound was prepared according to the procedure as described in example 1.

¹H-NMR (400 MHz, CD₃OD): δ 1.38 (t, J=7.0 Hz, 3H), 2.92 (dd, J=6.8, 13.6 Hz, 1H), 3.22-3.31 (m, 2H), 3.41-3.46 (m, 3H), 3.98-4.10 (m, 5H), 6.59 (d, J=8.0 Hz, 2H), 6.83 (d, J=8.0 Hz, 2H), 7.14 (d, J=8.0 Hz, 3H), 7.20 (d, J=8.0 Hz, 2H), 7.25 (br s, 1H), 7.36 (d, J=8.0 Hz, 1H).

MS (ES) m/z 516.1 (M+H).

### EXAMPLE 32

### (3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid

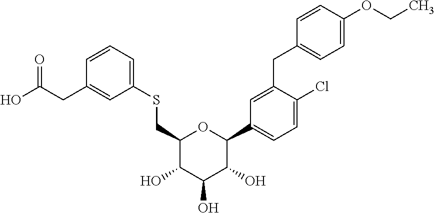

Title compound was prepared according to the procedure as described in example 2.

¹H-NMR (400 MHz, CD₃OD): δ 1.34 (t, J=7.2 Hz, 3H), 3.08 (dd, J=6.8 & 13.6 Hz, 1H), 3.21 (t, J=9.2 Hz, 1H), 3.38-3.53 (m, 6H), 3.92-4.04 (m, 5H), 6.78 (d, J=8.8 Hz, 2H), 7.01-7.1.7 (m, 6H), 7.28 (t, J=8.0 Hz, 3H).

MS (ES) m/z 576.3 (M+18).

### EXAMPLE 33

### (3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetic acid

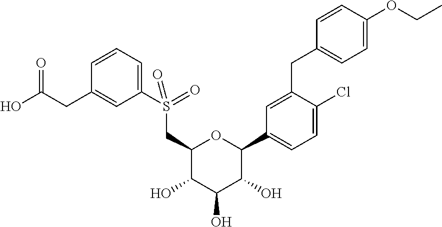

Title compound was prepared according to the procedure as described in example 2.

¹H-NMR (400 MHz, CD₃OD): δ 1.33 (t, J=7.2 Hz, 3H), 3.04 (t, J=9.2 Hz, 1H), 3.14 (t, J=9.2 Hz, 1H), 3.18-3.29 (m, 1H), 3.36 (t, J=9.2 Hz, 1H), 3.46-3.52 (m, 1H), 3.60-3.65 (m, 1H), 3.73 (t, J=9.6 Hz, 1H), 3.83 (s, 1H), 3.86 (d, J=5.2 Hz, 1H), 3.90 (s, 1H), 3.96 (q, J=7.2 Hz, 2H), 3.98 (d, J=6.8 Hz, 1H), 6.78-6.82 (m, 3H), 6.93 (d, J=2.0 Hz, 1H), 7.04-7.11 (m, 3H), 7.16 (d, J=8.0 Hz, 1H), 7.22 (d, J=8.0 Hz, 1H), 7.57 (d, J=8.0 Hz, 1H), 7.62 (s, 1H).

MS (ES) m/z 591.3 (M+H).

### EXAMPLE 34

### 2-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide

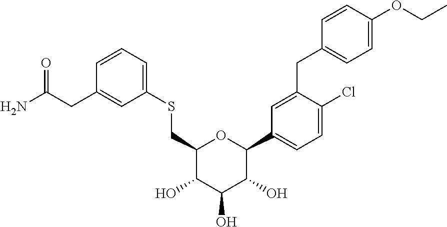

Title compound was prepared according to the procedure as described in example 3.

¹H-NMR (400 MHz, CD₃OD): δ 1.34 (t, J=7.2 Hz, 3H), 3.10 (dd, J=6.8, 14 Hz, 1H), 3.21 (t, J=8.8 Hz, 1H), 3.38-3.48 (m, 5H), 3.54 (t, J=9.6 Hz, 1H), 3.92-4.04 (m, 5H), 6.78 (d, J=8.4 Hz, 2H), 7.04-7.18 (m, 6H), 7.28 (app.t, J=8.4 Hz, 2H), 7.32 (s, 1H).

m/z 558.3 (M+H).

### EXAMPLE 35

### 2-(3-{(2S,3S,4R,5R,6S)-6-[4-chloro-3-(4-ethoxy-benzyl)-phenyl]-3,4,5-trihydroxy-tetrahydro-pyran-2-ylmethanesulfonyl}-phenyl)-acetamide

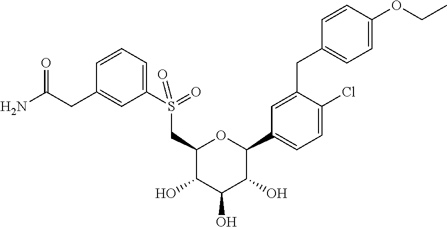

Title compound was prepared according to the procedure as described in example 3.

¹H-NMR (400 MHz, CD₃OD): δ 1.34 (t, J=7.2 Hz, 3H), 3.04 (t, J=9.2 Hz, 1H), 3.12-3.16 (m, 1H), 3.20 (d, J=11.2 Hz, 1H), 3.37 (t, J=8.8 Hz, 2H), 3.47-3.53 (m, 2H), 3.63 (d, J=14.0 Hz, 1H), 3.74 (t, J=9.6 Hz, 1H), 3.86 (t, J=9.2 Hz, 1H), 3.96 (q, J=7.2 Hz, 2H), 4.05 (d, J=14.8 Hz, 1H), 6.78-6.82 (m, 3H), 6.95 (d, J=2.0 Hz, 1H), 7.03 (app. t, J=8.0 Hz, 1H), 7.10 (d, J=8.8 Hz, 2H), 7.21 (app. t, J=8.0 Hz, 2H), 7.56 (d, J=7.6 Hz, 1H), 7.68 (s, 1H).

MS (ES) m/z 590.3 (M+H).

### EXAMPLE 36

### (3-{(2S,3S,4R,6R,6S)-6-[4-Chloro-3-(4-ethoxy-benzyl)-phenyl]-3,4,5-trihydroxy-tetrahydro-pyran-2-ylmethylsulfanyl}-4-fluoro-phenyl)-pyrrolidin-1-yl-methanone

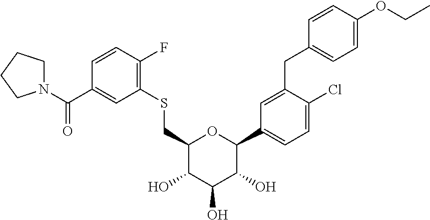

Title compound was prepared according to the procedure as described in example 3.

¹H-NMR (400 MHz, CD₃OD): δ 1.38 (t, J=7.2 Hz, 3H), 1.73-1.78 (m, 2H), 1.87-1.92 (m, 2H), 3.18-3.30 (m, 4H), 3.44 (t, J=8.8 Hz, 1H), 3.48-3.61 (m, 5H), 3.93-4.06 (m, 5H), 6.81 (d, J=8.8 Hz, 2H), 7.04 (d, J=8.4 Hz, 1H), 7.09 (d, J=8.8 Hz, 3H), 7.15 (s, 1H), 7.29 (d, J=8.0 Hz, 1H), 7.31-7.35 (m, 1H), 7.72 (dd, J=1.6 7.0 Hz, 1H).

MS (ES) m/z 616.1 (M+H).

### EXAMPLE 37

### Azetidin-1-yl-(3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-4-fluoro-phenyl)-methanone

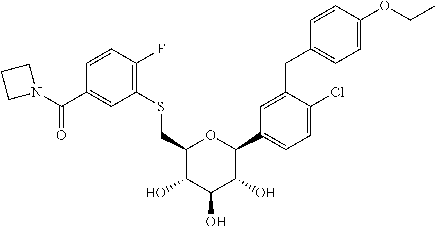

Title compound was prepared according to the procedure as described in example 3.

¹H-NMR (400 MHz, CD₃OD): δ 1.37 (t, J=7.2 Hz, 3H), 1.77-2.26 (m, 2H), 3.20-3.26 (m, 2H), 3.42-3.52 (m, 3H), 3.60-3.63 (m, 1H), 3.92-4.23 (m, 9H), 6.80 (d, J=8.8 Hz, 2H), 7.03-7.09 (m, 4H), 7.13 (s, 1H), 7.30 (d, J=8.4 Hz, 1H), 7.43-7.47 (m, 1H), 7.80 (dd, J=1.6 7.6 Hz, 1H).

MS (ES) m/z 602.0 (M+H).

### EXAMPLE 38

### 2-{(2S,3S,4R,5R,6S)-6-[4-Chloro-3-(4-ethoxy-benzyl)-phenyl]-3,4,5 trihydroxytetrahydro-pyran-2 ylmethylsulfanyl}-N-methyl benzamide

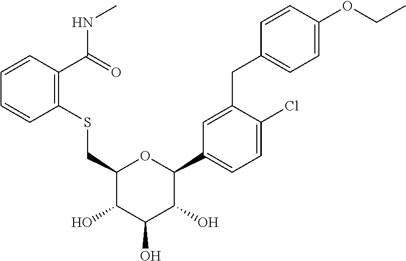

Title compound was prepared according to the procedure as described in example 3.

¹H-NMR (400 MHz, CD₃OD): δ 1.35 (t, J=7.2 Hz, 3H), 2.79 (s, 3H), 3.10 (dd, J=7.6, 13.6 Hz, 1H), 3.32-3.43 (m, 1H), 3.48-3.55 (m, 4H), 3.96-4.07 (m, 5H), 7.58 (d, J=7.6 Hz, 2H), 7.09 (d, J=10.4 Hz, 2H), 7.14-7.23 (m, 3H), 7.27-7.35 (m, 3H), 7.58 (d, J=7.6 Hz, 1H).

m/z 558.0 (M+H)

### EXAMPLE 39

### 2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-ethyl-benzamide

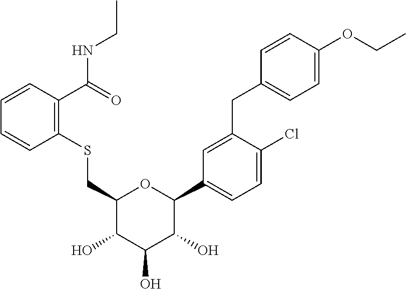

Title compound was prepared according to the procedure as described in example 3.

¹H-NMR (400 MHz, CD₃OD): δ 1.16 (t, J=7.6 Hz, 3H), 1.36 (t, J=7.2 Hz, 3H), 3.10 (dd, J=7.2, 13.6 Hz, 1H), 3.24-3.27 (m, 3H), 3.41-3.43 (m, 2H), 3.44-3.54 (m, 2H), 3.96-4.07 (m, 5H), 6.81 (d, J=8.4 Hz, 2H), 7.09 (d, J=8.4 Hz 2H), 7.13-7.22 (m, 3H), 7.27-7.33 (m, 3H), 7.58 (d, J=8.0 Hz, 1H).

m/z 572.1 (M+H).

### EXAMPLE 40

### 3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-methyl-benzamide

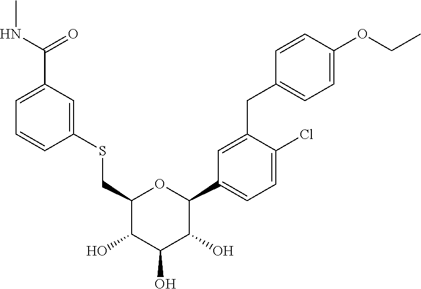

Title compound was prepared according to the procedure as described in example 3.

¹H-NMR (400 MHz, CD₃OD): δ 1.36 (t, J=7.2 Hz, 3H), 2.87 (s, 3H), 3.18-3.23 (m, 2H), 3.33-3.60 (m, 4H), 3.91-4.06 (m, 5H), 6.80 (d, J=8.4 Hz, 2H), 7.05 (d, J=8.4 Hz, 3H), 7.14 (s, 1H), 7.26-7.30 (m, 2H), 7.55 (d, J=8.0 Hz, 2H), 7.85 (s, 1H). MS (ES) m/z 558.1 (M+H).

### EXAMPLE 41

### 3-{(2S,3S,4R,5R,6S)-6-[4Chloro-3-(4-ethoxy-benzyl)-phenyl]-3,4,5-trihydroxy-tetrahydro-pyran-2-ylmethylsulfanyl}-N-ethyl-benzamide

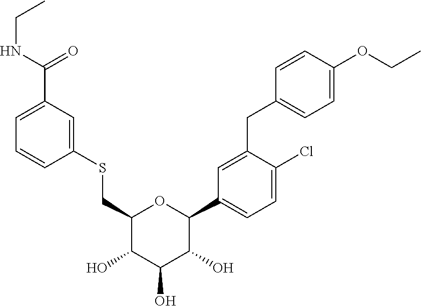

Title compound was prepared according to the procedure as described in example 3.

¹H-NMR (400 MHz, CD₃OD): δ 1.19 (t, J=7.2 Hz, 3H), 1.36 (t, J=7.2 Hz, 3H), 3.18-3.23 (m, 2H), 3.33-3.60 (m, 7H), 3.91-4.07 (m, 4H), 6.80 (d, J=8.4 Hz, 2H), 7.05 (d, J=8.4 Hz, 3H), 7.15 (d, J=1.6 Hz, 1H), 7.26-7.30 (m, 2H), 7.55 (t, J=8.0, 2H), 7.85 (s, 1H).

MS (ES) m/z 572.1 (M+H).

### EXAMPLE 42

### 4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-methyl-benzamide

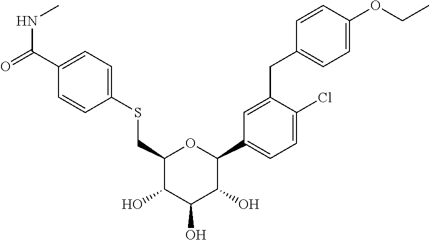

Title compound was prepared according to the procedure as described in example 3.

¹H-NMR (400 MHz, CD₃OD): δ 1.37 (t, J=6.8 Hz, 3H), 2.92 (s, 3H), 3.22 (t, J=8.4 Hz, 2H), 3.47 (qui, J=8.8 Hz, 2H), 3.57 (br d, J=14.5 Hz, 2H), 3.91-4.07 (m, 5H), 6.79 (d, J=8.0 Hz, 2H), 7.06 (d, J=8.0 Hz, 3H), 7.16 (s, 1H), 7.27 (d, J=8.0 Hz, 1H), 7.44 (d, J=8.0 Hz, 2H), 7.67 (d, J=8.0 Hz, 2H).

MS (ES) m/z 558.1 (M+H).

### EXAMPLE 43

### 4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide

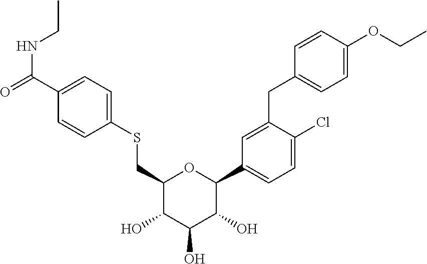

Title compound was prepared according to the procedure as described in example 3.

¹H-NMR (400 MHz, CD₃OD): δ 1.32 (t, J=7.0 Hz, 3H), 1.37 (t, J=6.8 Hz, 3H), 3.17-3.23 (m, 2H), 3.84-3.52 (m, 5H), 3.57-3.62 (m, 2H), 3.90-4.07 (m, 4H), 6.79 (d, J=8.0 Hz, 2H), 7.03-7.07 (m, 3H), 7.15 (d, J=0.8 Hz, 1H), 7.27 (d, J=8.0 Hz, 1H), 7.44 (d, J=8.0 Hz, 2H), 7.68 (d, J=8.0 Hz, 2H).

MS (ES) m/z 572.1 (M+H).

### EXAMPLE 44

### 4-{(2S,3S,4R,5R,6S)-6-[4-Chloro-3-(4-ethoxy-benzyl)-phenyl]-3,4,5-trihydroxy-tetrahydro-pyran2ylmethylsulfanyl}-N-ethyl-benzamide

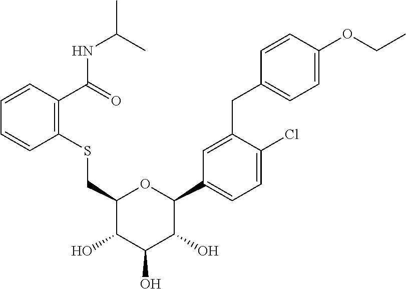

Title compound was prepared according to the procedure as described in example 3.

¹H-NMR (400 MHz, CD₃OD): δ 1.17-1.19 (m, 6H), 1.37 (t, J=7.2 Hz, 3H), 3.11 (dd, J=7.2, 13.6 Hz, 1H), 3.22-3.32 (m, 1H), 3.42-3.55 (m, 4H), 3.96-4.13 (m, 6H), 6.81 (d, J=8.4 Hz, 2H), 7.09 (d, J=8.4 Hz, 2H), 7.13-7.22 (m, 3H), 7.26-7.32 (m, 3H), 7.58 (d, J=8.0 Hz, 1H).

m/z 586.1 (M+H).

### EXAMPLE 45

### (2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1 yl-methanone

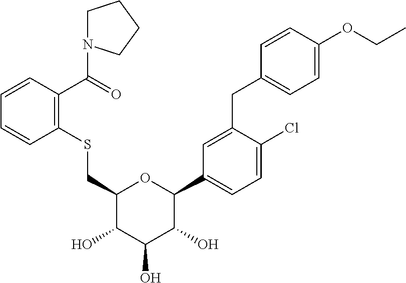

Title compound was prepared according to the procedure as described in example 3.

¹H-NMR (400 MHz, CD₃OD): δ 1.37 (t, J=6.8 Hz, 3H), 1.65-1.72 (m, 2H), 1.80-1.86 (m, 2H), 2.99-3.02 (m, 2H), 3.13-3.21 (m, 2H), 3.39-3.44 (m, 2H), 3.49-3.58 (m, 4H), 3.91-4.07 (m, 5H), 6.81 (d, J=8.8 Hz, 2H), 7.07 (br d, J=8.4 Hz, 3H), 7.15 (br d, J=5.6 Hz, 2H), 7.22 (t, J=6.8 Hz, 1H), 7.29 (d, J=8.0 Hz, 2H), 7.59 (d, J=8 Hz, 1H).

MS (ES) m/z 598.1 (M+H).

### EXAMPLE 46

### 3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide

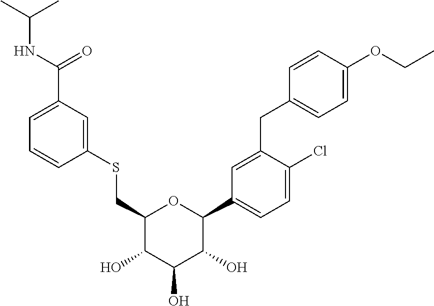

Title compound was prepared according to the procedure as described in example 3.

¹H-NMR (400 MHz, CD₃OD): δ 1.21-1.26 (m, 6H), 1.38 (t, J=7.2 Hz, 3H), 3.19-3.24 (m, 2H), 3.43-3.61 (m, 4H), 3.93-4.08 (m, 5H), 4.18-4.21 (m, 1H), 6.80 (d, J=8.4 Hz, 2H), 7.07 (d, J=8.4 Hz, 2H), 7.17 (s, 1H), 7.27-7.30 (m, 3H), 7.56 (t, J=8.4, 2H), 7.86 (s, 1H).

MS (ES) m/z 586.1 (M+H).

### EXAMPLE 47

### (3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-pyrrolidin-1 yl-methanone

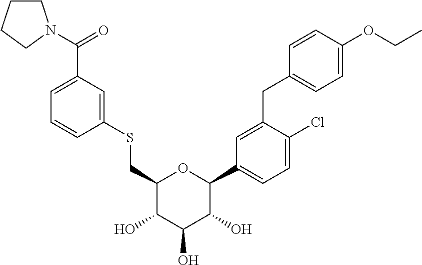

Title compound was prepared according to the procedure as described in example 3.

¹H-NMR (400 MHz, CD₃OD): δ 1.38 (t, J=6.8 Hz, 3H), 1.79-1.91 (m, 2H), 1.89-1.94 (m, 2H), 3.19-3.30 (m, 4H), 3.47 (t, J=8.8 Hz 1H), 3.51-3.60 (m, 5H), 3.94-4.03 (m, 4H), 4.07 (d, J=9.6 Hz, 1H), 6.81 (d, J=8.8 Hz, 2H), 7.08 (d, J=8.4 Hz, 2H), 7.18 (d, J=1.2 Hz, 1H), 7.26-7.33 (d, 3H), 7.51 (d, J=8.8 Hz, 2H), 7.58 (s, 1H).

MS (ES) m/z 598.1 (M+H).

### EXAMPLE 48

### 4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N-isopropyl-benzamide

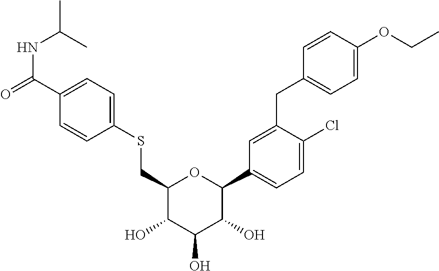

Title compound was prepared according to the procedure as described in example 3.

¹H-NMR (400 MHz, CD₃OD): δ 1.26 (d, J=6.4 Hz, 3H), 1.27 (d, J=6.4 Hz, 3H), 1.37 (t, J=7.2 Hz, 3H), 3.18-3.24 (m, 2H), 3.43-3.54 (m, 2H), 3.59-3.62 (m, 2H), 3.91-4.08 (m, 5H), 4.22 (qui, J=6.4 Hz, 1H), 6.80 (d, J=8.0 Hz, 2H), 7.04-7.09 (m, 3H), 7.17 (br s, 1H), 7.28 (d, J=8.0 Hz, 1H), 7.45 (d, J=8.0 Hz, 2H), 7.60 (d, J=8.0 Hz, 2H).

MS (ES) m/z 586.1 (M+H).

### EXAMPLE 49

### (4-{(2S,3S,4R,5R,6S)-6-[4Chloro-3-(4-ethoxy-benzyl)-phenyl]-3,4,5-trihydroxy-tetrahydro-pyran-2-ylmethylsulfanyl}-phenyl)-pyrrolidin-1 yl-methanone

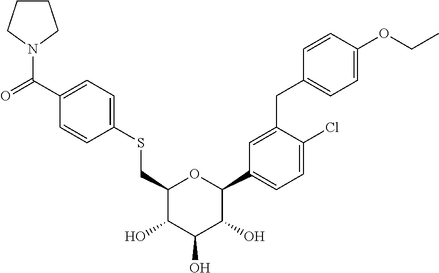

Title compound was prepared according to the procedure as described in example 3.

¹H-NMR (400 MHz, CD₃OD): δ 1.38 (t, J=6.8 Hz, 3H), 1.86-1.90 (m, 2H), 1.95-2.00 (m, 2H), 3.18-3.28 (m, 2H), 3.37-3.52 (m, 4H), 3.56-3.64 m, 4H), 3.96-4.09 (m, 5H), 6.81 (d, J=8.4 Hz, 2H), 7.09-7.14 (m, 3H), 7.24 (br s, 1H), 7.31 (d, J=8.0 Hz, 1H), 7.37 (d, J=8.4 Hz, 2H), 7.45 (d, J=8.4 Hz, 2H).

MS (ES) m/z 598.1

### EXAMPLE 50

### 3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide

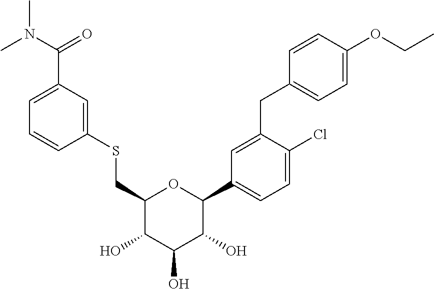

Title compound was prepared according to the procedure as described in example 3.

¹H-NMR (400 MHz, CD₃OD): δ 1.38 (t, J=7.2 Hz, 3H), 2.83 (s, 3H), 3.04 (s, 3H), 3.18-3.33 (m, 2H), 3.44-3.53 (m, 3H), 3.56-3.60 (m, 1H), 3.95-4.08 (m, 5H), 6.81 (d, J=8.4 Hz, 2H), 7.09-7.12 (m, 3H), 7.16 (d, J=7.6 Hz, 1H), 7.2 (d, J=1.6 Hz 1H), 7.3 (m, 2H), 7.45 (s, 1H) 7.50 (d, J=8.0, 1H).

MS (ES) m/z 572.1 (M+H).

### EXAMPLE 51

### 4-{(2S,3S,4R,5R,6S)-6-[4-Chloro-3-(4-ethoxy-benzyl)phenyl]-3,4,5-trihydroxy-tetrahydro-pyran-2-ylmethylsulfanyl}-N,N-dimethyl-benzamide

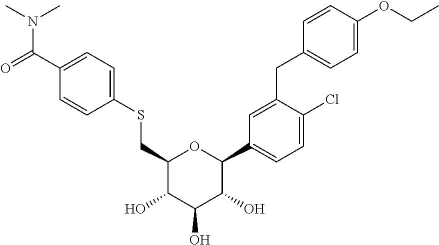

Title compound was prepared according to the procedure as described in example 3.

¹H-NMR (400 MHz, CD₃OD): δ 1.38 (t, J=6.8 Hz, 3H), 2.95 (s, 3H), 2.09 (s, 3H), 3.21-3.26 (m, 2H), 3.45-3.49 (m, 2H), 3.55-3.61 (m, 2H), 3.98-4.09 (m, 5H), 6.82 (d, J=8.0 Hz, 2H), 7.07 (m, 3H), 7.24 (br s, 1H), 7.27 (d, J=8.0 Hz, 2H), 7.32 (d, J=8.0 Hz, 1H), 7.45 (d, J=8.0 Hz, 2H).

MS (ES) m/z 572.1 (M+H).

### EXAMPLE 52

### 2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-N,N-dimethyl-benzamide

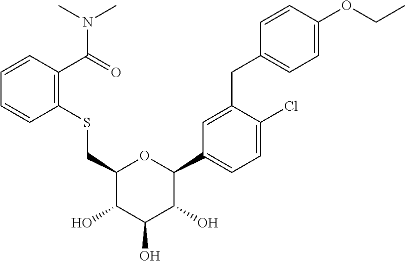

Title compound was prepared according to the procedure as described in example 3.

¹H-NMR (400 MHz, CD₃OD): δ 1.38 (t, J=6.8 Hz, 3H), 2.7 (s, 3H), 3.03 (s, 3H), 3.15-3.26 (m, 2H), 3.42-3.44 (m, 2H), 3.52-3.55 (m, 2H), 3.95-4.08 (m, 5H), 6.82 (d, J=8.4 Hz, 2H), 7.08-7.18 (m, 5H), 7.21-7.32 (m, 3H), 7.60 (d, J=8.0 Hz, 1H).

m/z 572.0(M+H)

### EXAMPLE 53

### 4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide

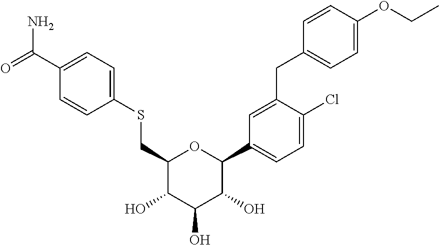

Title compound was prepared according to the procedure as described in example 3.

¹H-NMR (400 MHz, CD₃OD): δ 1.38 (t, J=7.0 Hz, 3H), 3.19-3.25 (m, 2H), 3.34-3.50 (m, 2H), 3.53-3.64 (m, 2H), 3.96-4.09 (m, 5H), 6.80 (d, J=8.0 Hz, 2H), 7.07 (d, J=8.0 Hz, 3H), 7.18 (d, J=1.2 Hz, 1H), 7.30 (d, J=8.0 Hz, 1H), 7.45 (d, J=8.0 Hz, 2H), 7.75 (d, J=8.0 Hz, 2H).

MS (ES) ink 544.1 (M+H).

### EXAMPLE 54

### Cyclopentanecarboxylic acid (3-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide

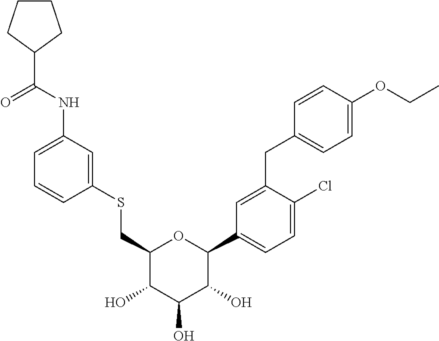

Title compound was prepared according to the procedure as described in example 25.

¹H-NMR (400 MHz, CD₃OD): δ 1.37 (t, J=6.8 Hz, 3H), 1.64 (m, 2H), 1.78 (m, 4H), 1.90 (m, 2H), 2.76 (m, 1H), 3.13-3.22 (m, 2H), 3.33-3.60 (m, 4H), 3.91-4.08 (m, 5H), 6.80 (d, J=8.4 Hz, 2H), 7.061 (m, 6H), 7.27 (d, J=8.0 Hz, 1H), 7.61 (d, J=8.0 Hz, 1H), 7.68 (s, 1H).

MS (ES) m/z 613.1 (M+2).

### EXAMPLE 55

### N-(3-{(2S,3S,4R,5R,6S)-6-[4-Chloro-3-(4-ethoxy-benzyl)-phenyl]-3,4,6-trihydroxy-tetrahydro-pyran-2-ylmethylsulfanyl}-phenyl)-benzamide

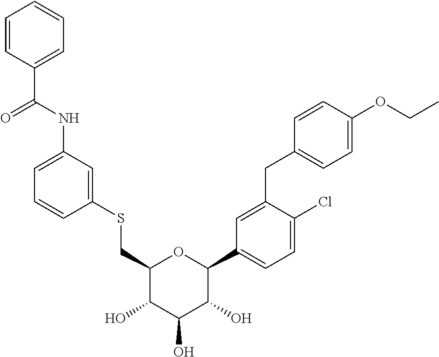

Title compound was prepared according to the procedure as described in example 25.

¹H-NMR (400 MHz, CD₃OD): δ 1.36 (t, J=7.2 Hz, 3H), 3.16-3.25 (m, 2H), 3.33-3.62 (m, 4H), 3.90-4.09 (m, 5H), 6.78 (d, J=7.2 Hz, 2H), 7.05 (d, J=8.8 Hz, 2H), 7.09-7.11 (dd, J=1.6, 8.0 Hz, 1H), 7.18-7.28 (m, 4H), 7.52 (d, J=8.0 Hz, 3H), 7.60 (t, J=7.2 Hz, 1H), 7.83 (s, 1H), 7.89 (d, J=7.2 Hz, 2H).

MS (ES) m/z 620.1 (M+2).

### EXAMPLE 56

### Cyclohexanecarboxylic acid (3-{(2S,3S,4R,5R,6S)-6-[4-chloro-3-(4-ethoxy-benzyl)-phenyl]-3,4,5-trihydroxy-tetrahydro-pyran-2ylmethylsulfanyl}-phenyl)-amide

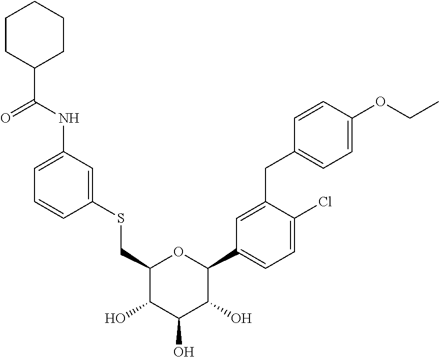

Title compound was prepared according to the procedure as described in example 25.

¹H-NMR (400 MHz, CD₃OD): δ 1.29-1.04 (m, 5H), 1.48-1.52 (m, 2H), 1.73 (br d, J=12.0 Hz, 2H), 1.83 (br d, J=12.0 Hz, 4H), 2.30-2.33 (m, 1H), 3.14-3.22 (m, 2H), 3.45 (t, J=8.8 Hz, 1H), 3.51-3.55 (m, 2H), 3.58-3.60 (m, 1H), 3.91-4.08 (m, 5H), 6.80 (d, J=8.4 Hz, 2H), 7.06 (d, J=8.4 Hz, 3H), 7.11-7.17 (m, 3H), 7.28 (d, J=8.0 Hz, 1H), 7.36 (d, J=8.0 Hz, 1H), 7.68 (br s, 1H).

MS (ES) m/z 627.2 (M+2)

### EXAMPLE 57

### N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide

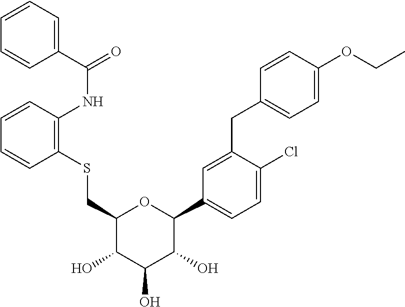

Title compound was prepared according to the procedure as described in example 25.

¹H-NMR (400 MHz, CD₃OD): δ 1.36 (t, J=7.2 Hz, 3H), 2.97-2.99 (m, 1H), 3.14-3.16 (m, 2H), 3.52 (d, J=13.6 Hz, 2H), 3.86-3.90 (m, 4H), 3.97 (q, J=6.8 Hz, 2H), 6.78 (d, J=8.8 Hz, 2H), 6.97-7.04 (m, 4H), 7.15-7.18 (m, 2H), 7.35 (t, J=7.6 Hz, 3H), 7.54 (t, J=7.6 Hz, 1H), 7.67 (d, J=7.6 Hz, 1H), 7.81 (d, J=7.2 Hz, 2H), 8.12 (d, 8.0 Hz, 1H).

MS (ES) m/z 620.1

### EXAMPLE 58

### N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-benzamide

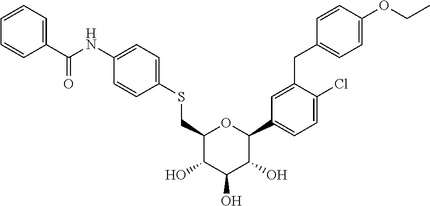

Title compound was prepared according to the procedure as described in example 25.

¹H-NMR (400 MHz, DMSO-d₆): δ 1.29 (t, J=6.8 Hz, 3H), 3.01-3.11 (m, 2H), 3.27 (br s, 2H), 3.41-3.47 (m, 2H), 3.91-4.04 (m, 5H), 4.93 (d, J=5.6 Hz, 1H), 5.07 (d, J=4.0 Hz, 1H), 5.27 (d, J=4.0 Hz, 1H), 6.82 (d, J=8.8 Hz, 2H), 7.09 (d, J=8.8 Hz, 2H), 7.13 (s, 1H), 7.24 (s, 1H), 7.34-7.37 (m, 3H), 7.53-7.61 (m, 3H), 7.72 (d, J=8.8 Hz, 2H), 7.94 (d, J=8.0 Hz, 2H), 10.28 (s, 1H).

MS (ES) m/z 620.0 (M+H).

### EXAMPLE 59

### Cyclohexanecarboxylic acid (4-{(2S,3S,4R,5R,6S)-6-[4-chloro3-(4-ethoxy-benzyl)-phenyl]-3,4,5-trihydroxy-tetrahydro-pyran-2-ylmethylsulfanyl}-phenyl)-amide

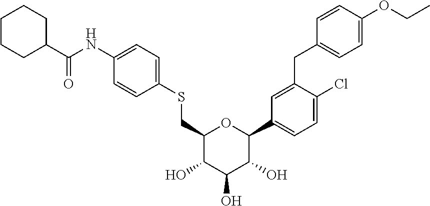

Title compound was prepared according to the procedure as described in example 25.

¹H-NMR (400 MHz, CD₃OD): δ 1.32-1.40 (m, 6H), 1.53-1.56 (m, 2H), 1.76 (br d, J=12.0 Hz, 1H), 1.88 (br t, J=13.6 Hz, 4H), 2.35-2.38 (m, 1H), 3.11 (dd, J=6.4, 14.0, Hz, 1H), 3.20 (t, J=9.2 Hz, 1H), 3.41-3.55 (m, 4H), 3.93-4.06 (m, 5H), 6.81 (d, J=8.4 Hz, 2H), 7.02 (dd, J=1.6, 8.4 Hz, 1H), 7.08 (d, J=8.4 Hz, 2H), 7.16 (s, 1H), 7.28 (d, J=8.4 Hz, 1H), 7.36 (d, J=8.8 Hz, 2H), 7.46 (d, J=8.8 Hz, 2H). MS (ES) m/z 626.1,

### EXAMPLE 60

### Cyclopentanecarboxylic acid (4-{(2S,3S,4R,5R,6S)-6-[4-chloro-3-(4-ethoxybenzyl)-phenyl]-3,4,5-trihydroxy-tetrahydro-pyran-2-ylmethylsulfanyl}-phenyl)-amide

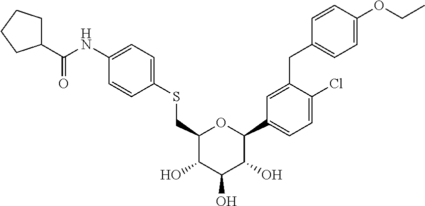

Title compound was prepared according to the procedure as described in example 25.

¹H-NMR (400 MHz, CD₃OD): δ 1.37 (t, J=7.2 Hz, 3H), 1.67 (br s, 2H), 1.81-1.87 (m, 4H), 1.96 (br s, 2H), 2.8 (dd, J=6.8, 14.4 Hz, 1H), 3.18 (t, J=8.8 Hz, 2H), 3.41-3.55 (m, 4H), 3.93-4.06 (m, 5H), 6.81 (d, J=8.4 Hz, 2H), 7.03 (dd, J=2.0, 8.4 Hz, 1H), 7.08 (d, J=8.4 Hz, 2H), 7.16 (s, 1H), 7.28 (d, J=8.4 Hz, 1H), 7.37 (d, J=8.4 Hz, 2H), 7.46 (d, J=8.4 Hz, 2H).

MS (ES) m/z 612.1

### EXAMPLE 61

### N-(4-{(2S,3S,4R,6R,6S)-6-[4-Chloro-3-(4-ethoxy-benzyl)-phenyl]-3,4,6-trihydroxy-tetrahydro-pyran-2-ylmethylsulfanyl}-phenyl)-acetamide

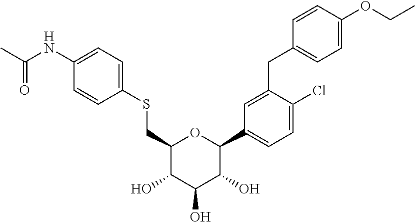

Title compound was prepared according to the procedure as described in example 25.

¹H-NMR (400 MHz, CD₃OD): δ 1.38 (t, J=6.8 Hz, 3H), 2.13 (s, 3H), 3.08 (dd, J=6.8 & 14.4, Hz, 1H), 3.21 (t, J=9.2 Hz, 1H), 3.41-3.57 (m, 4H), 3.95-4.07 (m, 5H), 6.81 (d, J=8.8 Hz, 2H), 7.07-7.09 (m, 3H), 7.17 (s, 1H), 7.30 (d, J=8.0 Hz, 1H), 7.36 (d, J=9.2 Hz, 2H), 7.43 (d, J=8.8 Hz, 2H). MS (ES) m/z 558.0 (M+H).

### EXAMPLE 62

### N-(2-{(2S,3S,4R5R6S)-6-[4-Chloro-3-(4-ethoxy-benzyl)-phenyl]-3,4,5-trihydroxy-tetrahydro-pyran-2-ylmethylsulfanyl}-phenyl)-acetamide

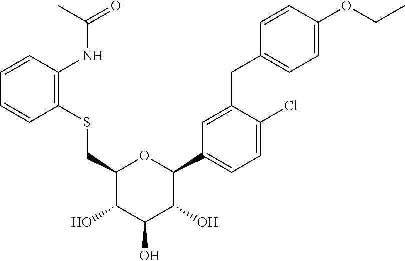

Title compound was prepared according to the procedure as described in example 25.

¹H-NMR (400 MHz, DMSO-d₆): δ 1.30 (t, J=6.8 Hz, 3H), 1.97 (s, 3H), 2.97 (dd, J=8.0, 13.2 Hz, 1H), 3.10-3.13 (m, 1H), 3.22-3.27 (m, 3H), 3.41-3.43 (m, 1H), 3.92-4.04 (m, 5H), 4.95 (d, J=5.6 Hz, 1H), 5.11 (d, J=4.8 Hz, 1H), 5.33 (d, J=4.8 Hz, 1H), 6.83 (d, J=8.8 Hz, 2H), 7.09-7.12 (m, 3H), 7.13-7.19 (m, 2H), 7.27 (d, J=1.6 Hz, 1H), 7.37 (d, J=8.4 Hz, 1H), 7.45 (dd, J=1.2 & 7.6 Hz, 1H), 7.51-7.53 (d, J=7.6 Hz, 1H), 9.21 (s, 1H).

m/z 558.0(M+H)

### EXAMPLE 63

### Cyclohexanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide

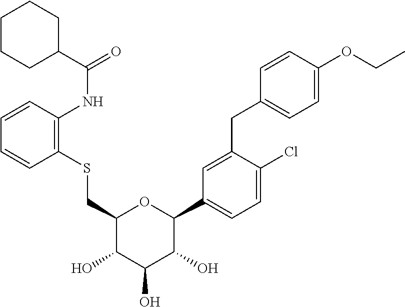

Title compound was prepared according to the procedure as described in example 25.

¹H-NMR (400 MHz, DMSO-d₆): δ 1.16-1.24 (m, 4H), 1.28-1.36 (m, 5H), 1.59 (d, J=10 Hz, 1H), 1.68 (d, J=8.0 Hz, 2H), 1.78 (d, J=12.8 Hz, 2H), 2.26-2.25 (m, 1H), 2.96 (dd, J=8.4, 13.6 Hz, 1H), 3.08-3.09 (m, 1H), 3.17-3.26 (m, 2H), 3.31-3.42 (m, 1H), 3.85-4.05 (m, 5H), 4.94 (d, J=6.0 Hz, 1H), 5.15 (d, J=4.4 Hz, 1H), 5.31 (d, J=4.8 Hz, 1H), 6.82 (d, J=8.8 Hz, 2H), 7.09 (d, J=8.4 Hz, 3H), 7.15-7.19 (m, 2H), 7.26 (s, 1H), 7.36 (d, J=8.4 Hz, 1H), 7.47 (d, J=7.6 Hz, 1H), 7.53 (d, J=8.0 Hz, 1H), 9.03 (s, 1H).

m/z 626.2(M+H)

### EXAMPLE 64

### Cyclopentanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide

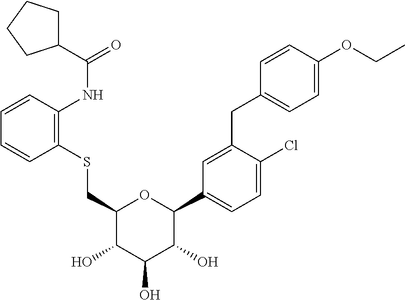

Title compound was prepared according to the procedure as described in example 25.

¹H-NMR (400 MHz, DMSO-d₆): δ 1.29 (t, J=7.2 Hz, 3H), 1.41-1.49 (m, 2H), 1.55-1.82 (m, 6H), 2.64-2.75 (m, 1H), 2.96 (dd, J=8.4, 13.6 Hz, 1H), 3.09 (q, J=2.4 Hz, 1H), 3.21-3.26 (m, 2H), 3.31-3.42 (m, 2H), 3.92-4.03 (m, 5H), 4.94 (d, J=6 Hz, 1H), 5.15 (d, J=4.4 Hz, 1H), 5.31 (d, J=4.8 Hz, 1H), 6.82 (d, J=8.4 Hz, 2H), 7.09 (d, J=8 Hz, 3H), 7.15-7.20 (m, 2H), 7.26 (s, 1H), 7.36 (d, J=8.0 Hz, 1H), 7.47 (d, J=8.0 Hz, 1H), 7.56 (d, J=8.0 Hz, 1H), 9.06 (s, 1H).

m/z 612.1(M+H)

### EXAMPLE 65

### Cyclopropanecarboxylic acid (2-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide

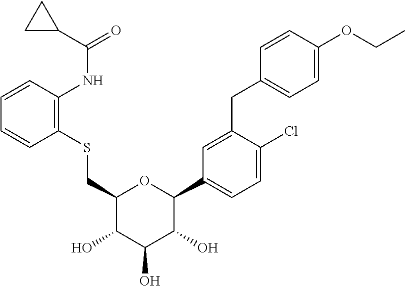

Title compound was prepared according to the procedure as described in example 25.

¹H-NMR (400 MHz, DMSO-d₆): δ 0.70-0.76 (m, 4H), 1.35 (t, J=6.8 Hz, 3H), 1.72-1.82 (m, 1H), 3.00 (dd, J=7.2, 13.6 Hz, 1H), 3.10-3.16 (m, 1H), 3.25-3.29 (m, 3H), 3.42-3.49 (m, 1H), 3.93-4.06 (m, 5H), 4.95 (d, J=5.2 Hz, 1H), 5.11 (br s, 1H), 5.33 (br s, 1H), 6.83 (d, J=8.0 Hz, 2H), 7.09-7.19 (m, 5H), 7.28 (s, 1H), 7.38 (d, J=8.4 Hz, 1H), 7.46-7.53 (m, 2H), 9.42 (s, 1H).

m/z 584.0(M+H)

### EXAMPLE 66

### Cyclopropanecarboxylic acid (3-{(2S,3S,4R,5R,6S)-6-[4-chloro-3-(4-ethoxy-benzyl)-phenyl]-3,4,6-trihydroxy-tetrahydro-pyran-2-ylmethylsulfanyl}-phenyl)-amide

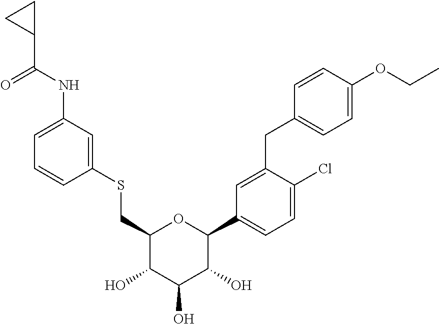

Title compound was prepared according to the procedure as described in example 25.

¹H-NMR (400 MHz, DMSO-d₆): δ 0.78-0.80 (m, 4H), 1.30 (t, J=7.2 Hz, 3H), 1.74-1.77 (m, 1H), 3.02-3.09 (m, 2H), 3.26-3.28 (m, 2H), 3.41-3.49 (m, 2H), 3.90-4.05 (m, 5H), 4.95 (d, J=6.0 Hz, 1H), 5.09 (d, J=4.4 Hz, 1H), 5.29 (d, J=4.8 Hz, 1H), 6.29 (d, J=8.4 Hz, 2H), 7.01 (d, J=7.6 Hz, 1H), 7.08-7.20 (m, 4H), 7.245 (s, 1H), 7.35 (d, J=8.4 Hz, 2H), 7.63 (s, 1H), 10.21 (s, 1H).

MS (ES) m/z 584.0 (M+H).

### EXAMPLE 67

### N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide

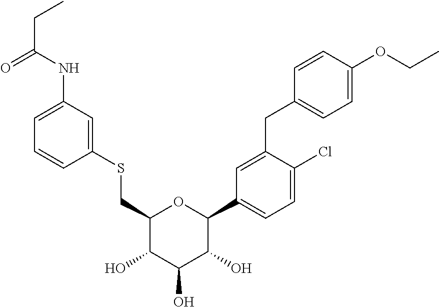

Title compound was prepared according to the procedure as described in example 25.

¹H-NMR (400 MHz, DMSO-d₆): δ 1.07 (t, J=7.2 Hz, 3H), 1.30 (t, J=7.2 Hz, 3H), 2.27-2.33 (m, 2H), 3.02-3.10 (m, 2H), 3.26-3.28 (m, 2H), 3.41-3.49 (m, 2H), 3.91-4.05 (m, 5H), 4.94 (d, J=6.0 Hz, 1H), 5.08 (d, J=4.4 Hz, 1H), 5.28 (d, J=4.8 Hz, 1H), 6.83 (d, J=8.4 Hz, 2H), 7.01 (d, J=8.0 Hz, 1H), 7.08-7.20 (m, 4H), 7.25 (s, 1H), 7.36 (t, J=8.4 Hz, 2H), 7.63 (s, 1H), 9.86 (s,

MS (ES) m/z 572.0 (M+H).

### EXAMPLE 68

### (2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamic acid methyl ester

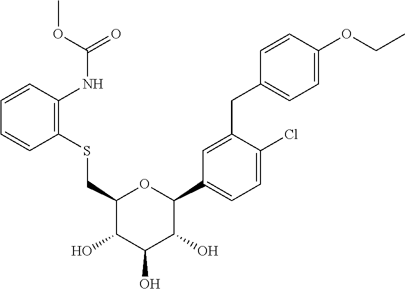

Title compound was prepared according to the procedure as described in example 25.

¹H-NMR (400 MHz, DMSO-d₆): δ 1.30 (t, J=6.8 Hz, 3H), 2.96 (dd, J=8.0, 13.6 Hz, 1H), 3.11 (q, J=5.2 Hz, 1H), 3.21-3.26 (m, 2H), 3.31-3.40 (m, 2H), 3.57 (s, 3′-1) 3.93-4.03 (m, 5H), 4.95 (d, J=6.0 Hz, 1H), 5.11 (d, J=4.4 Hz, 1H), 5.31 (d, J=4.8 Hz, 1H), 6.83 (d, J=8.4 Hz, 2H), 7.11 (d, J=8.8 Hz, 3H), 7.15-7.20 (m, 2H), 7.26 (s, 1H), 7.37 (d, J=8.0 Hz, 1H), 7.46-7.49 (m, 2H), 8.59 (s, 1H).

m/z 574.0(M+H)

### EXAMPLE 69

### (3-{(2S,3S,4R,5R,6S)-6-[4-Chloro-3-(4-ethoxy-benzyl)-phenyl]-3,4,6-trihydroxy-tetrahydro-pyran-2-ylmethylsulfanyl}-phenyl)-carbamic acid methyl ester

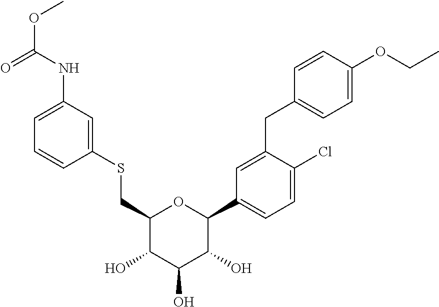

Title compound was prepared according to the procedure as described in example 25.

¹H-NMR (400 MHz, DMSO-d₆): δ 1.30 (t, J=7.2 Hz, 3H), 3.02-3.12 (m, 2H), 3.25-3.28 (m, 2H), 3.41-3.49 (m, 2H), 3.66 (s, 3H), 3.91-4.05 (m, 5H), 4.95 (d, J=5.6 Hz, 1H), 5.10 (d, J=4.0 Hz, 1H), 5.30 (d, J=4.4 Hz, 1H), 6.83 (d, J=8.4 Hz, 2H), 6.97 (d, J=8.0 Hz, 1H), 7.08-7.20 (m, 4H), 7.25 (d, J=1.6 Hz, 2H), 7.35 (d, J=8.0 Hz, 1H) 7.46 (s, 1H) 9.67 (s, 1H).

MS (ES) m/z 574.0 (M+H).

### EXAMPLE 70

### N-(4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide

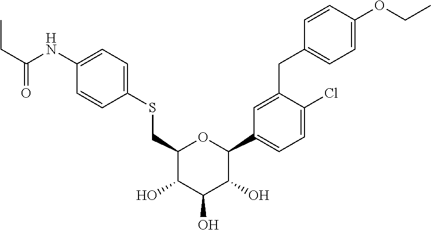

Title compound was prepared according to the procedure as described in example 25.

¹H-NMR (400 MHz, DMSO-d₆): δ 1.09 (t, J=7.2 Hz, 3H), 1.31 (t, J=6.8 Hz, 3H), 2.31 (q, J=7.2 Hz, 2H), 3.01 (dd, J=7.2 & 14.4, Hz, 1H), 3.09 (t, J=9.2 Hz, 1H), 3.26 (br d, J=3.6 Hz, 2H), 3.39-3.45 (m, 2H), 3.91-4.03 (m, 5H), 4.93 (d, J=6.0 Hz, 1H), 5.07 (d, J=3.6 Hz, 1H), 5.27 (d, J=4.4 Hz, 1H), 6.83 (d, J=8.4 Hz, 2H), 7.09 (d, J=8.4 Hz, 2H), 7.24 (s, 1H), 7.28 (d, J=8.4 Hz, 2H), 7.35 (d, J=8.4 Hz, 2H), 7.52 (d, J=8.8 Hz, 2H), 9.89 (s, 1H). MS (ES) m/z 572.0 (M+H).

### EXAMPLE 71

### (4-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-carbamicacid methyl ester

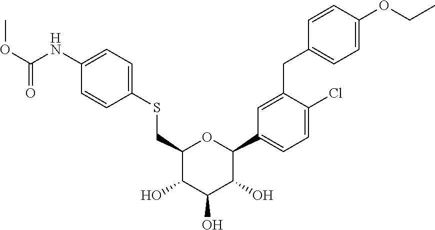

Title compound was prepared according to the procedure as described in example 25.

¹H-NMR (400 MHz, DMSO-d₆): δ 1.31 (t, J=6.8 Hz, 3H), 2.99 (dd, J=, 7.2 & 14.4 Hz, 1H), 3.09 (t, J=9.2 Hz, 1H), 3.26 (br s, 2H), 3.81-3.40 (m, 2H), 3.67 (s, 3H), 3.95-4.03 (m, 5H), 4.93 (d, J=6.4 Hz, 1H), 5.07 (d, J=4.4 Hz, 1H), 5.26 (d, J=4.4 Hz, 1H), 6.83 (d, J=8.4 Hz, 2H), 7.08-7.12 (m, 3H), 7.24 (s, 1H), 7.28 (d, J=8.4 Hz, 2H), 7.34-7.4 (m, 3H), 9.69 (s, 1H).

MS (ES) m/z 574.0 (M+H).

### EXAMPLE 72

### N-(2-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-propionamide

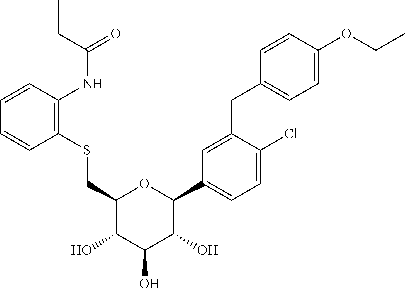

Title compound was prepared according to the procedure as described in example 25.

¹H-NMR (400 MHz, DMSO-d₆): δ 1.00 (t, J=6.0 Hz, 3H), 1.22 (t, J=6.8 Hz, 3H), 2.19-2.24 (m, 2H), 2.95 (dd, J=8.0, 13.6 Hz, 1H), 3.05-3.14 (m, 1H), 3.20-3.25 (m, 2H), 3.30-3.42 (m, 2H), 3.91-4.02 (m, 6H), 4.92 (d, J=6.0 Hz, 1H), 5.10 (d, J=4.4 Hz, 1H), 5.31 (d, J=5.2 Hz, 1H), 6.81 (d, J=8.4 Hz, 2H), 7.08 (d, J=8.8 Hz, 2H), 7.14-7.19 (m, 2H), 7.25 (s, 1H), 7.36 (d, J=8.4 Hz, 1H), 7.45 (d, J=7.6 Hz, 1H), 7.55 (d, J=7.6 Hz, 1H), 9.09 (s, 1H). m/z 572.0(M+H)

### EXAMPLE 73

### Cyclopropanecarboxylic acid (4-{(2S,3S,4R,5R,6S)-6-[4-chloro-3-(4-ethoxy-benzyl)-phenyl]-3,4,5-trihydroxy-tetrahydro-pyran-2-ylmethylsulfanyl}-phenyl)-amide

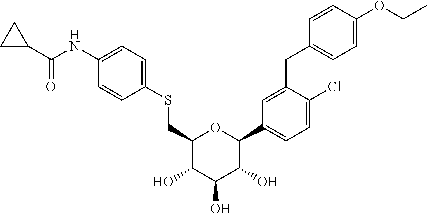

Title compound was prepared according to the procedure as described in example 25.

¹H-NMR (400 MHz, CD₃OD): δ 0.87-0.89 (m, 2H), 0.98 (t, J=4.0 Hz, 2H), 1.37 (t, J=7.2 Hz, 3H), 1.71 (br s, 1H), 3.13 (dd, J=6.8, 14.4, Hz, 1H), 3.21 (t, J=9.2 Hz, 1H), 3.41-3.55 (m, 4H), 3.98-4.07 (m, 5H), 6.81 (d, J=8.0 Hz, 2H), 7.05 (s, 1H), 7.08 (d, J=8.4 Hz, 2H), 7.16 (s, 1H), 7.30 (d, J=8.4 Hz, 1H), 7.36 (d, J=8.8 Hz, 2H), 7.45 (d, J=8.8 Hz, 2H). MS (ES) m/z 584.0

### EXAMPLE 74

### (2S,3R,4R,5S,6S)-2-[4-Chloro-3-(4-ethoxy-benzyl)-phenyl]-6-[4-(2-hydroxy ethyl)benzenesulfonylmethyl]-tetrahydro-pyran-3,4,5-triol

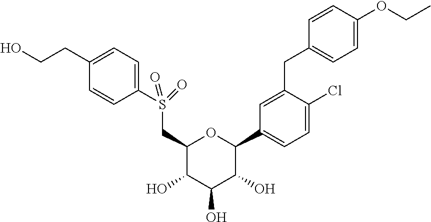

Title compound was prepared according to the procedure as described in example 5.

¹H-NMR (400 MHz, CD₃OD): δ 1.35 (t, J=6.8 Hz, 3H), 2.53-2.58 (m, 2H), 3.08-3.17 (m, 2H), 3.38 (t, J=9.2 Hz, 2H), 3.44 (d, J=4.8 Hz, 1H), 3.58 (t, J=6.8 Hz, 2H), 3.75 (t, J=10.8 Hz, 1H), 3.84 (d, J=9.6 Hz, 1H), 3.90-4.07 (m, 5H), 6.80-6.84 (m, 3H), 6.94 (d, J=8.4 Hz, 2H), 7.01 (d, J=1.6 Hz, 1H), 7.14 (d, J=8.8 Hz, 2H), 7.22 (d, J=8.0 Hz, 1H), 7.57 (d, J=8.0 Hz, 1H).

MS (ES) m/z 577.3 (M+H).

### EXAMPLE 75

### (2S,3R,4R,5S,6S)-2-[4-Chloro-3-(4-ethoxy-benzyl)-phenyl]-6-[3-(2-hydroxy-ethyl)benzenesulfonylmethyl]-tetrahydro-pyran-3,4,5-triol

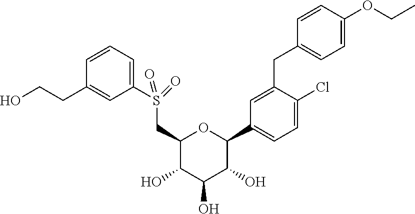

Title compound was prepared according to the procedure as described in example 5.

¹H-NMR (400 MHz, CD₃OD): δ 1.35 (t, J=7.2 Hz, 3H), 2.47-2.53 (m, 2H), 3.07 (t, J=9.2 Hz, 1H), 3.14 (t, J=9.6 Hz, 1H), 3.36 (t, J=8.8 Hz, 1H), 3.49-3.52 (m, 1H), 3.56 (t, J=6.8 Hz, 2H), 3.62 (d, J=14 Hz, 1H), 3.74 (t, J=9.6 Hz, 1H),3.82 (s, 1H), 3.85 (d, J=6.8 Hz, 1H), 3.98 (q, J=6.8 Hz, 2H), 4.04 (d, J=15.2 Hz, 1H), 6.80 (d, J=8.8 Hz, 3H), 6.92 (d, J=1.6 Hz, 1H), 7.07-7.10 (m, 3H), 7.14 (d, J=8.0 Hz, 1H), 7.21 (d, J=8.40 Hz, 1H), 7.55 (d, J=6.4 Hz, 2H).

MS (ES) m/z 577.3 (M+H).

### EXAMPLE 76

### (2S,3R,4R,6S,6S)-2-[4-Chloro-3-(4-ethoxy-benzyl)-phenyl]-6-[3-(2-hydroxy-ethyl)phenylsulfanylmethyl]-tetrahydro-pyran-3,4,5-triol

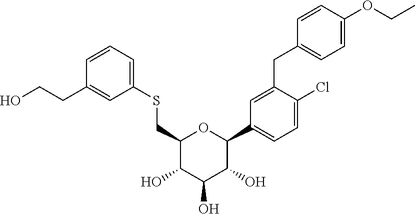

Title compound was prepared according to the procedure as described in example 5.

¹H-NMR (400 MHz, CD₃OD): δ 1.34 (t, J=6.8 Hz, 3H), 2.69 (t, J=7.2 Hz, 2H), 3.06 (dd, J=6.8 Hz, J=14 Hz, 1H), 3.20 (t, J=9.2 Hz, 1H), 3.30-3.53 (m, 4H), 3.66 (t, J=6.8 Hz, 2H), 3.92-4.04 (m, 5H), 6.78 (d, J=8.4 Hz, 2H), 6.98 (d, J=7.2 Hz, 1H), 7.04-7.13 (m, 4H), 7.17 (s, 1H), 7.21 (d, J=8.0 Hz, 1H), 7.24 (s, 1H), 7.28 (d, J=8.0 Hz, 1H).

MS (ES) m/z 562.3 (M+18).

### EXAMPLE 77

### (2S,3R,4R,5S,6S)-2-[4-Chloro-3-(4-ethoxy-benzyl)-phenyl]-6-(3-hydroxymethyl-phenylsulfanylmethyl)-tetrahydro-pyran-3,4,5-triol

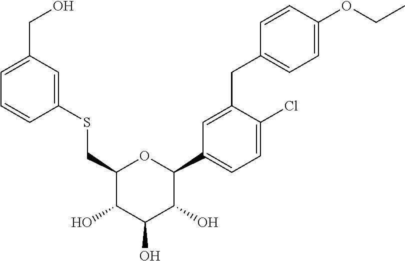

Title compound was prepared according to the procedure as described in example 5.

¹H-NMR (400 MHz, CD₃OD): δ 1.37 (t, J=6.8 Hz, 3H), 3.13 (dd, J=14.0 Hz & 7.2 Hz, 1H), 3.25 (t, J=8.8 Hz, 3.44-3.57 (m, 4H), 3.96-4.04 (m, 4H), 4.07 (d, J=9.6 Hz, 1H), 4.51 (s, 2H), 6.82 (d, J=8.8 Hz, 2H), 7.09 (d, J=8.8 Hz, 2H), 7.13 (d, J=8.0 Hz, 2H), 7.18-7.22 (m, 2H), 7.30-7.38 (m, 2H), 7.40 (s, 1H).

MS (ES+) m/z 548.3 (M+18).

### EXAMPLE 78

### 3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-benzamide

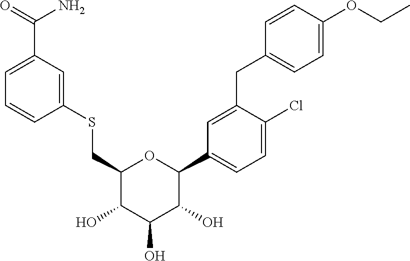

Title compound was prepared according to the procedure as described in example 3.

¹H-NMR (400 MHz, CD₃OD): δ 1.37 (t, J=7.2 Hz, 3H), 3.18-3.25 (m, 2H), 3.42-3.60 (m, 4H), 3.94-4.07 (m, 5H), 6.81 (d, J=8.8 Hz, 2H), 7.07-7.10 (m, 3H), 7.17 (d, J=2.0 Hz, 1H), 7.27-7.31 (m, 2H), 7.57 (d, J=8.4 Hz, 1H), 7.62 (d, J=8.4 Hz, 1H), 7.91 (s, 1H).

MS (ES+) m/z 544.3 (M+1).

### EXAMPLE 79

### 3-{(2S,3S,4R,5R,6S)-6-[4-Chloro-3-(4-ethoxy-benzyl)-phenyl]-3,4,5-trihydroxy-tetrahydro-pyran-2-ylmethanesulfonyl}-benzamide

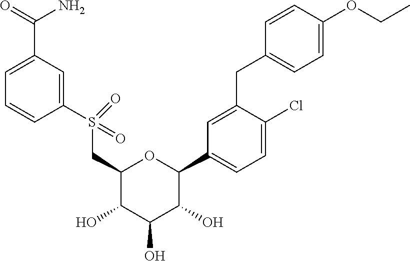

Title compound was prepared according to the procedure as described in example 6.

¹H-NMR (400 MHz, CD₃OD): δ 1.38 (t, J=7.2 Hz, 3H), 3.07 (t, J=8.8 Hz, 1H), 3.17 (t, J=8.8 Hz, 1H), 3.40 (t, J=8.8 Hz, 1H), 3.56-3.62 (m, 1H), 3.68-3.72 (m, 1H), 3.78-3.84 (m, 2H), 3.89-3.93 (m, 1H), 3.99-4.07 (m, 3H), 6.76 (d, J=8.4 Hz, 1H), 6.83-6.85 (m, 3H), 7.10-7.16 (m, 3H), 7.21 (d, J=8.0 Hz, 1H), 7.76 (d, J=8.0 Hz, 1H), 7.83 (d, J=8.0 Hz, 1H), 8.28 (s, 1H).

MS (ES+) m/z 576.3(M+1).

### EXAMPLE 80

### N-(3-{(2S,3S,4R,5R,6S)-6-[4-Chloro-3-(4-ethoxy-benzyl)-phenyl]-3,4,5-trihydroxy-tetrahydro-pyran-2-ylmethylsulfanyl}-phenyl)-acetamide

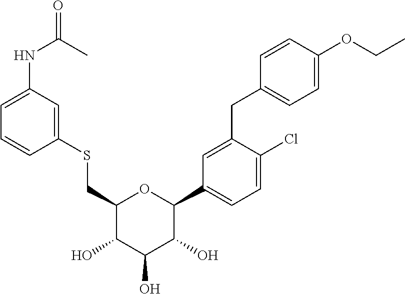

Title compound was prepared according to analogous procedure as described in example 25.

¹H-NMR (400 MHz, CD₃OD): δ 1.33 (t, J=6.8 Hz, 3H), 2.05 (s, 3H), 3.12 (dd, J=14.0 Hz & 6.8 Hz, 1H), 3.18 (t, J=9.2 Hz, 1H), 3.39-3.57 (m, 4H), 3.89-3.98 (m, 4H), 4.03 (d, J=9.2 Hz, 1H), 6.77 (d, J=8.4 Hz, 2H), 7.03-7.08 (m, 3H), 7.10-7.14 (m, 3H), 7.26 (d, J=8.0 Hz, 1H), 7.30-7.31 (m, 1H), 7.60 (s, 1H).

MS (ES+) m/z 558.3 (M+1).

### EXAMPLE 81

### N-(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-acetamide

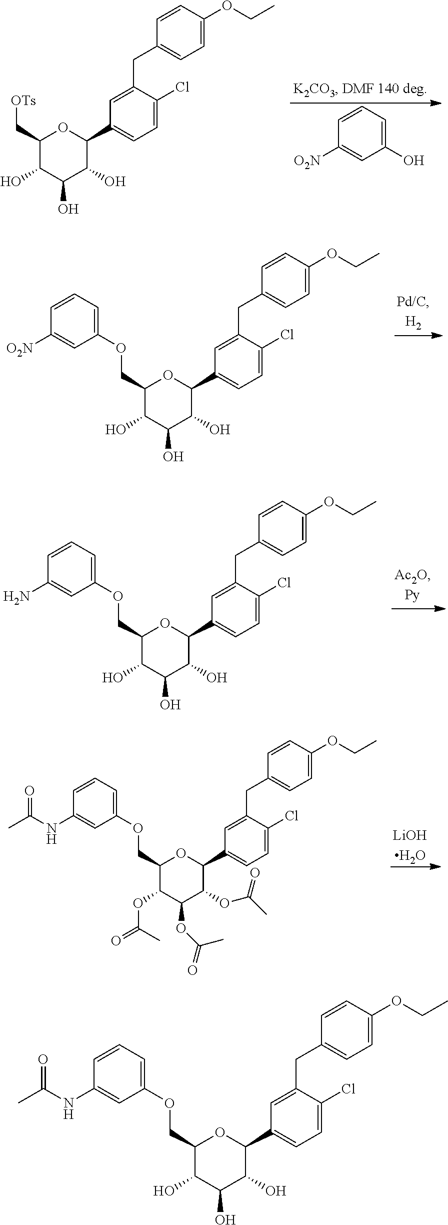

Step I. To a mixture of toluene-4-sulfonic acid (2R,3S,4R,5R,6S)-6-[4-chloro-3-(4-ethoxy-benzyl)-phenyl]-3,4,5-trihydroxy-tetrahydro-pyran-2-ylmethyl ester (150 mg, 0.26 mmole) in dimethylformamide (3 mL), 3-nitrophenol (74 mg, 0.53 mmole), potassium carbonate (150 mg, 1.06 mmole) were added and stirred at 130° C. for 12 hours. The reaction mixture was diluted with water (25 mL) and extracted with dichloromethane (4×25 mL), resulting organic layer washed with brine (25 mL).The product obtained after the removal of solvent furnished (2S,3R,4R,5S,6R)-2-[4-chloro-3-(4-ethoxy-benzyl)-phenyl]-6-(3-nitro-phenoxymethyl)-tetrahydro-pyran-3,4,5 triol (120 mg).

Step II. To a mixture of (2S,3R,4R,5S,6R)-2-[4-chloro-3-(4-ethoxy-benzyl)-phenyl]-6-(3-nitro-phenoxymethyl)-tetrahydro-pyran-3,4,5-triol (120 mg, 0.22 mmole) methanol (5 mL), Pd/C 10% on carbon (30 mg) was added and reaction mixture was stirred under hydrogen atmosphere at room temperature for 10 hours. The reaction mixture was filtered through celite bed, filtrate was evaporated to furnish crude 2S,3R,4R,5S,6R)-2-[4-Chloro-3-(4-ethoxy-benzyl)-phenyl]-6-(3-amino-phenoxymethyl)-tetrahydro-pyran-3,4,5-triol (90 mg).

Step III. To a solution of 2S,3R,4R,5S,6R)-2-[4-chloro-3-(4-ethoxy-benzyl)-phenyl]-6-(3-amino-phenoxymethyl)-tetrahydro-pyran-3,4,5-triol (90 mg, 1.8 mmole) in pyridine (0.5 mL), acetic anhydride (0.1 mL), DMAP (1 mg) was added at room temperature and stirred overnight. The reaction mixture was diluted with water (10 mL) and acidified with 1N HCl, extracted with dichloromethane (2×20 mL). The product obtained after the removal of solvent furnished ccetic acid (2R,3R,4R,5S,6S)-4,5-diacetoxy-2-(3-acetylamino-phenoxymethyl)-6-[4-chloro-3-(4-ethoxy-benzyl)-phenyl]-tetrahydro-pyran-3-yl ester (150 mg).

Step IV. To a solution of acetic acid (2R,3R,4R,5S,6S)-4,5-diacetoxy-2-(3-acetylamino-phenoxymethyl)-6-[4-chloro-3-(4-ethoxy-benzyl)-phenyl]-tetrahydro-pyran-3-yl ester (445 mg, 0.67 mmole) in THF: MeOH: H₂O (3:1:2, 13 mL), lithium hydroxide (56 mg, 1.33 mmole) was added at room temperature and stirred overnight. The reaction mixture was diluted with water (10 mL) and extracted with ethyl acetate (3×20 mL). The crude product obtained after the removal of solvent was purified using preparative HPLC to furnish N-(3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-acetamide (14 mg).

¹H-NMR (400 MHz, CD₃OD): δ 1.37 (t, J=7.2 Hz, 3H), 2.11 (s, 3H), 3.28-3.30 (m, 1H), 3.50 (t, J=89.2 Hz, 1H), 3.59 (t, J=9.2 Hz, 1H), 3.65-3.71 (m, 1H), 3.96-4.03 (m, 4H), 4.15 (d, J=9.6 Hz, 1H), 4.20 (t, J=5.6 Hz, 1H), 4.34 (d, J=10 Hz, 1H), 6.73 (d, J=6.4 Hz, 1H), 6.80 (d, J=8.4 Hz, 2H), 7.06-7.10 (m, 3H), 7.18 (d, J=8.4 Hz, 1H), 7.23-7.28 (m, 2H), 7.32-7.35 (m, 2H). MS (ES) m/z 542.2 (M+H).

### EXAMPLE 82

### 3-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide

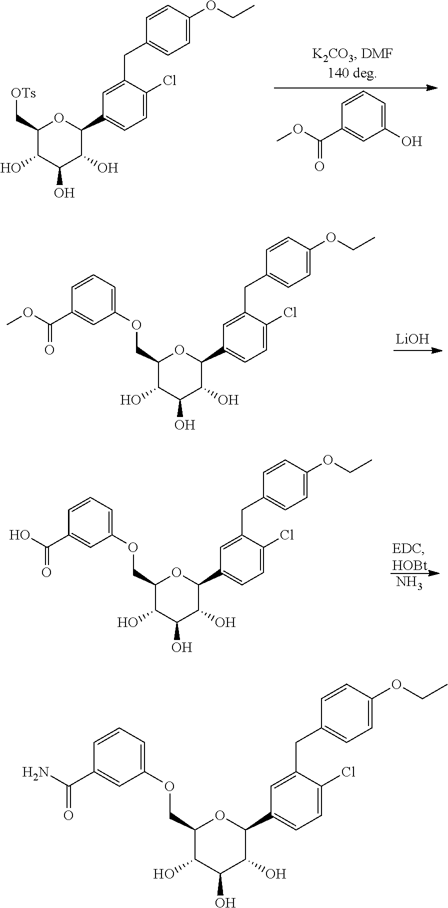

Step I. To a mixture of Toluene-4-sulfonic acid (2R,3S,4R,5R,6S)-6-[4-chloro-3-(4-ethoxy-benzyl)-phenyl]-3,4,5-trihydroxy-tetrahydro-pyran-2-ylmethyl ester (700 mg, 1.24 mmole) in dimethylformamide (3 mL), 3-hydroxymethylbenzoate (377 mg, 2.48 mmole), potassium carbonate (685 mg, 4.96 mmole) were added and stirred at 130° C. for 12 hrs. The reaction mixture was diluted with water (25 mL) and extracted with dichloromethane (4×25 mL), resulting organic layer washed with brine (25 mL).The product obtained after the removal of solvent furnished 3-{(2R,3S,4R,5R,6S)-6-[4-chloro-3-(4-ethoxy-benzyl)-phenyl]-3,4,5-trihydroxy-tetrahydro-pyran-2-ylmethoxy}-benzoic acid methyl ester (350 mg).

Step II. To a mixture of 3-{(2R,3S,4R,5R,6S)-6-[4-chloro-3-(4-ethoxy-benzyl)-phenyl]-3,4,5-trihydroxy-tetrahydro-pyran-2-ylmethoxy}-benzoic acid methyl ester (33 mg, 0.06 mmole) in THF:MeOH:H₂O (3 mL), lithium hydroxide (4 mg, 0.12 mmole) was added at room temperature and stirred overnight. The reaction mixture was diluted with water (10 mL) and neutralized with 1N dilute HCl (0.2 mL) extracted with ethyl acetate (3×20 mL). The crude product obtained after the removal of solvent to furnish 3-{(2R,3S,4R,5R,6S)-6-[4-Chloro-3-(4-ethoxy-benzyl)-phenyl]-3,4,5-trihydroxy-etrahydro-pyran-2-ylmethoxy}-benzoic acid (30 mg).

Step III. To a solution of 3-{(2R,3S,4R,5R,6S)-6-[4-chloro-3-(4-ethoxy-benzyl)-phenyl]-3,4,5-trihydroxy-etrahydro-pyran-2-ylmethoxy}-benzoic acid (100 mg, 0.18 mmole) in DMF (1 mL), ammonia in tetrahydrofuran (1 mL), HOBt (30 mg, 0.22 mmole) was added and stirred for 10 minutes, EDCl (43 mg, 0.22 mmole) was added and stirred at room temperature overnight. The reaction mixture was diluted with water (10 mL) and extracted with ethyl acetate (2×20 mL) The crude product obtained after the removal of solvent was purified using preparative HPLC to furnish the title compound (21 mg).

¹H-NMR (400 MHz, CD₃OD): δ 1.37 (t, J=7.2 Hz, 3H), 3.29-3.31 (m, 1H), 3.52 (t, J=8.8 Hz, 1H), 3.61 (t, J=9.2 Hz, 1H), 3.73 (dd, J=4.0 & 9.6 Hz, 1H), 3.96-4.07 (m, 4H), 4.15 (d, J=10.4 Hz, 1H), 4.24 (dd, J=5.6 Hz, J=10.8 Hz, 1H), 4.42 (d, J=9.6 Hz, 1H), 6.80 (d, J=8.4 Hz, 2H), 7.09 (d, J=8.4 Hz, 2H), 7.16 (dd, J=1.60 Hz, J=8.4 Hz, 1H), 7.23 (dd, J=2.0 Hz, J=8.0 Hz, 1H), 7.27 (s, 1H), 7.34-7.38 (m, 2H), 7.44-7.50 (m, 2H).

MS (ES) m/z 528.0 (M+H).

### EXAMPLE 83

### 3-{(2R,3S,4R,5R,6S)-6-[4-Chloro-3-(4-ethoxy-benzyl)-phenyl]-3,4,5-trihydroxy-tetrahydro-pyran-2-ylmethoxy}-N-methyl-benzamide

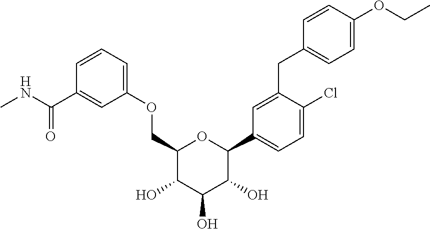

To the solution of 3-{(2R,3S,4R,5R,6S)-6-[4-chloro-3-(4-ethoxy-benzyl)-phenyl]-3,4,5-trihydroxy-tetrahydro-pyran-2-ylmethoxy}-benzoic acid methyl ester (100 mg, 0.18 mmole) in 2M methanolic methylammine (2.0 mL), 1,5,7 triazo bicycle[4,4,0] dec-5-ene (25 mg, 0.18 mmole) was added and heated in sealed tube at 90° C. for 24 hours. The reaction mixture was concentrated and purified by preparative HPLC to furnish the title compound (7 mg).

¹H-NMR (400 MHz, CD₃OD): δ 1.37 (t, J=7.2 Hz, 3H), 2.95 (s, 3H), 2.91-3.30 (m, 1H), 3.51 (t, J=9.2 Hz, 1H), 3.60 (t, J=9.2 Hz, 1H), 3.72 (dd, J=3.6 & 8.0 Hz, 1H), 3.96-4.07 (m, 4H), 4.15 (d, J=9.2 Hz, 1H), 4.25 (dd, J=5.2 & 10.8 Hz, 1H), 4.32 (d, J=10.8 Hz, 1H), 6.80 (d, J=8.4 Hz, 2H), 7.06 (d, J=8.4 Hz, 2H), 7.15 (d, J=8.0 Hz, 1H), 7.22-7.26 (m, 2H), 7.33-7.40 (m, 3H), 7.44 (s, 1H).

MS (ES) m/z 542.1 (M+H).

Examples 84 and 85 were prepared in an analogous procedure as described in example

### EXAMPLE 84

### (3-{(2R,3S,4R,5R,6S)-6-[4-Chloro-3-(4-ethoxy-benzyl)-phenyl]-3,4,5-trihydroxy-tetrahydro-pyran-2-ylmethoxy}-phenyl)-pyrrolidin-1-yl-methanone

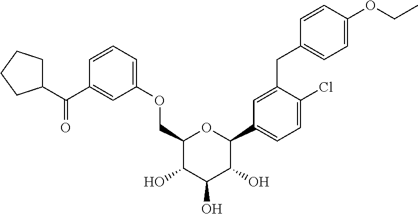

¹H-NMR (400 MHz, CD₃OD): δ 1.38 (t, J=7.2 Hz, 3H), 1.87 (q, J=6.4 & 13.2 Hz, 2H), 1.95 (q, J=6.8 & 14.0 Hz, 2H), 3.28-3.31 (r, 1H), 3.33-3.41 (m, 2H), 3.51 (t, c=8.8 Hz, 1H), 3.56-3.61 (m, 3H), 3.71 (dd, J=4.0 & 9.2 Hz, 1H), 3.97-4.08 (m, 4H), 4.14 (d, J=9.6 Hz, 1H), 4.25 (dd, J=5.6 & 11.2 Hz, 1H), 4.41 (d, J=10.0 Hz, 1H), 6.80 (d, J=8.8 Hz, 2H), 7.07-7.3 (m, 6H), 7.23 (d, J=8.4 Hz, 1H), 7.35 (d, J=7.6 Hz, 2H)

(ES) m/z 582.0 (M+H).

### EXAMPLE 85

### Azetidin-1-yl-(3-{(2R,3S,4R,5R,6S)-6-[4-chloro-3-(4-ethoxy-benzyl)-phenyl]-3,4,6-trihydroxy-tetrahydro-pyran-2-ylmethoxy}-phenyl)-methanone

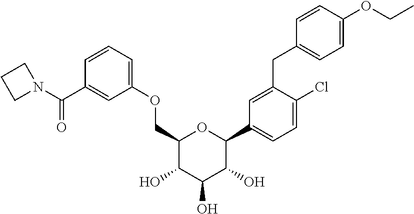

¹H-NMR (400 MHz, CD₃OD): δ 1.38 (t, J=7.2 Hz, 3H), 2.29-2.33 (m, 2H), 3.29-3.31 (m, 1H), 3.49 (t, J=8.8 Hz, 1H), 3.56 (t, J=9.2 Hz, 1H), 3.69-3.72 (m, 1H), 3.97-4.07 (m, 4H), 4.13-4.33 (m, 6H), 4.48 (d, J=11.2 Hz, 1H), 6.80 (d, J=8.8 Hz, 2H), 7.09 (d, J=8.8 Hz, 2H), 7.12 (dd, J=2.0 & 8.4 Hz, 1H), 7.19-7.26 (m, 4H), 7.34-7.38 (m, 2H)

MS (ES) m/z 568.1 (M+H).

### EXAMPLE 86

### 4-{(2R,3S,4R,5R,6S)-6-[4-Chloro-3-(4-ethoxy-benzyl)-phenyl]-3,4,5-trihydroxy-tetrahydro-pyran-2-ylmethoxy}-benzamide

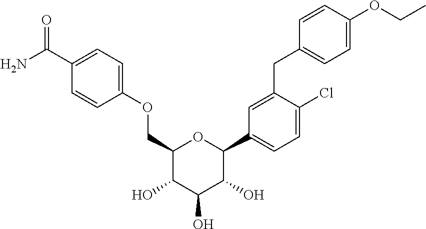

The title compound was prepared in an analogous procedure as described in example 120.

¹H-NMR (400 MHz, CD₃OD): δ 1.37 (t, J=6.8 Hz, 3H), 3.28-3.30 (m, 1H), 3.51 (t, J=8.8 Hz, 1H), 3.59 (t, J=8.8 Hz, 1H), 3.70-3.73 (m, 1H), 3.96-4.03 (m, 4H), 4.12 (d, J=9.6 Hz, 1H), 4.25 (dd, J=5.6 & 11.2 Hz, 1H), 4.42 (d, J=11.6 Hz, 1H), 6.79 (d, J=8.8 Hz, 2H), 7.05 (d, J=8.8 Hz, 2H), 7.10 (d, J=8.4 Hz, 2H), 7.23 (d, J=8.4 Hz, 1H), 7.26 (S, 1H), 7.35 (d, J=8.0 Hz, 1H), 7.85 (d, J=8.8 Hz, 2H).

MS (ES) m/z 528.3(M+H).

### EXAMPLE 87

### In Vitro Assays

The inhibitory effect on the sodium-dependent glucose cotransporter SGLT, SGLT1 and SGLT2, of compounds of formula I may be demonstrated using the following test procedures:

The ability of the substances to inhibit the SGLT-2 activity may be demonstrated in a test set-up in which a CHO-K1 cell line (ATCC No. CCL 6 1) or alternatively an HEK293 cell line (ATCC No. CRL-1573), which is stably transfected with an expression vector pZeoSV (Invitrogen, EMBL accession number L36849), which contains the cDNA for the coding sequence of the human sodium glucose cotransporter 2 (Genbank Ace. No.NM_{—}003041) (CHO-hSGLT2 or HEK-hSGLT2). These cell lines transport 14 C-labelled alpha-methyl-glucopyranoside (14 C-AMG, Amersham) into the interior of the cell in sodium-dependent manner.

The SGLT-2 assay is carried out as follows: CHO-hSGLT2 cells are cultivated in Ham's F12 Medium (BioWhittaker) with 10% foetal calf serum and 250 μg/mL zeocin (Invitrogen), and HEK293-hSGLT2 cells are cultivated in DMEM medium with 10% foetal calf serum and 250 μg/mL zeocin (Invitrogen). The cells are detached from the culture flasks by washing twice with PBS and subsequently treating with trypsin/EDTA. After the addition of cell culture medium the cells are centrifuged, resuspended in culture medium and counted in a Casy cell counter. Then 40,000 cells per well are seeded into a white, 96-well plate coated with poly-D-Iysine and incubated overnight at 37° C., 5% CO2. The cells are washenwice with 250 μl of assay buffer (Hanks Balanced Salt Solution, 137 mM NaCl, 5.4 mM KCl, 2.8 mM CaCl2, 1.2 mM MgSO4 and 10 mM HEPES (pH 7.4), 50 μg/mL of gentamycin). 250 μl of assay buffer and 5 μl of test compound are then added to each well and the plate is incubated for a further 15 minutes in the incubator. 5 μl of 10% DMSO are used as the negative control. The reaction is started by adding 5 μl of 14 C-AMG (0.05 μCi) to each well. After 2 hours' incubation at 37° C., 5% CO2, the cells are washed again with 250 μl of PBS (200C) and then lysed by the addition of 25 μl of 0.1 N NaOH (5 min. at 37° C.). 200 μl of MicroScint20 (Packard) are added to each well and incubation is continued for a further 20 min at 37° C. After this incubation the radioactivity of the 14 C-AMG absorbed is measured in a Topcount (Packard) using a 14 C scintillation program.

To determine human SGLT1 inhibitory activity, an analogous test was set up in which the cDNA for hSGLTI (Genbank Ace. No. NM000343) instead of hSGLT2 cDNA is expressed in CHO-K1 or HEK293 cells. The uptake assay buffer in the case of the hSGLT1 assay contains 10 mM HEPES, 5 mM Tris, 140 mM NaCl, 2 mM KCl, 1 mM CaCl₂, and 1 mM MgCl₂, pH 7.4 containing 0.5 mM of α-methyl-D-glucopyranoside (AMG), 10 μM of [¹⁴C]-α-methyl-D-glucopyranoside and different inhibitor concentrations.

The compounds according to the invention may for example have IC₅₀ values for SGLT2 inhibition below 1000 nM, particularly below 100 nM, most preferably below 10 nM. The compounds according to the invention may also have SGLT1 inhibitory activity.

The title compounds of the above Examples were evaluated in the above described assay and the results of which are collated in Table 1.

TABLE 1

| | SGLT2 IC₅₀ nM | SGLT1 IC₅₀ nM |
|---|---|---|
| Example Number | (n = 1-4) | (n = 1-4) |
| 8 | 85 | — |
| 10 | 20 | <1000 |
| 11 | 60 | <1000 |
| 15 | 65 | — |
| 23 | 36 | — |
| 24 | 57 | — |
| 25 | 7 | <50 |
| 26 | 12 | <50 |
| 27 | 6 | <50 |
| 36 | 6 | — |
| 37 | 3 | <50 |
| 40 | 13 | <50 |
| 47 | 14 | <50 |
| 50 | 8 | <50 |
| 61 | 28 | — |
| 67 | 9 | — |
| 69 | 28 | — |
| 77 | 61 | — |
| 78 | 42 | <100 |
| 80 | 13 | <100 |
| 83 | 72 | — |
| 84 | 11 | — |
| 85 | 9 | — |

It can be seen that the compounds of the invention are useful as inhibitors of SGLT2 and therefore useful in the treatment of diseases and conditions mediated by SGLT2 such as the metabolic disorders disclosed herein.

It will be understood that the invention has been described by way of example only and modifications may be made whilst remaining within the scope and spirit of the invention.

We claim:

1. A compound of formula I: 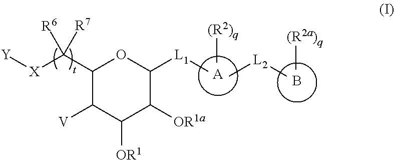
or a pharmaceutically acceptable salt thereof, wherein
Rings A and B are independently C₆₋₁₀aryl, C₃₋₇cycloalkyl, heteroaryl or heterocyclic;
L₁ is —S(O)ₚ—, —N(R³)—, or —(CH₂)ₙ—, provided that L₁ is not —N(R³)— when X is —O—;
L₂ is —(CH₂)ₙ—O—(CH₂)ₘ—, —S(O)ₚ—, —N(R³)—, —Si(R′)(R″)—, —(C(R′)(R″))ₙ—, —(CH₂)ₙC(O)(CH₂)ₘ—, —(CH₂)ₙC(O)NR³(CH₂)ₘ—, —(CH₂)ₙNR³C(O)(CH₂)ₘ—, —C₂₋₆alkenyl-, —C(O) C₂₋₆alkenyl-, —N(R³)C(O)N(R³)—, —N(R³)SO₂—, or —SO₂N(R³)—;
V is halogen, OR^{1b} or hydrogen;
with the proviso that, when V is —OR^{1b}, Y is C₆₋₁₀aryl, L₁ is bond, L₂ is —CH₂—, rings A and B are phenyl, then Y is not unsubstituted aryl or an aryl that is substituted exclusively with halogen, C₁₋₆haloalkyl, C₁₋₆perhaloalkyl, C₁₋₆alkoxy, C₁₋₆haloalkoxy, C₁₋₆perhaloalkoxy or cyano as substituents;
t is an integer from 1-4;
m, for each occurrence, is independently, 0 or an integer from 1-4;
n, for each occurrence, is independently, 0 or an integer from 1-4;
p, for each occurrence, is independently, 0 or an integer from 1-2;
R′ and R″, for each occurrence, are independently hydrogen, halogen, C₁₋₆alkyl, or C₁₋₆perhaloalkyl or taken together form a cyclic ring which may optionally have heteroatoms selected from O, N or S;
R¹, R^{1a} and R^{1b} are independently selected from hydrogen, C₁₋₆alkyl, C₆₋₁₀arylC₁₋₄ alkyl, —C(O)C₆₋₁₀aryl or —C(O)C₁₋₆alkyl;
R² and R^{2a}, for each occurrence, are independently halogen, hydroxy, C₁₋₄hydroxylalkyl, cyano, —NR⁴R⁵, —CH₂NR⁴R⁵, C₁₋₄alkyl, C₃₋₇cycloalkyl, C₁₋₄alkoxy, C₃₋₇cycloalkoxy, —S(O)ₚR³, —S(O)₂NR⁴R⁵, —OS(O)₂R³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₃₋₇cycloalkyl, C₃₋₇cycloalkylC₁₋₄alkyl, C₆₋₁₀aryl, C₆₋₁₀arylC₁₋₄alkyl, C₆₋₁₀aryloxy, heterocyclyl, heterocyclylC₁₋₄alkyl, heteroaryl, heteroarylC₁₋₄alkyl, heteroaryloxy, or heterocycloxy;
R³ is hydrogen, C₁₋₆alkyl, C₃₋₇ cycloalkyl, C₆₋₁₀aryl, heteroaryl, —NR⁴R⁵ or heterocyclyl;
q, for each occurrence, is independently 0 or an integer from 1-3;
Y is C₆₋₁₀aryl, C₃₋₇cycloalkyl, heteroaryl, or heterocyclic, each of which may be optionally substituted;
X is S(O)ₚ or O;
R⁴ and R⁵, for each occurrence, are each independently hydrogen, C₁₋₆ alkyl, C₃₋₇ cycloalkyl, C₃₋₇cycloalkylC₁₋₄alkyl, C₆₋₁₀arylC₁₋₄alkyl, C₆₋₁₀aryl, heteroaryl, heteroarylC₁₋₄alkyl, heterocyclyl, or heterocyclylC₁₋₄alkyl, or
R⁴ and R⁵ taken together may form a monocyclic or a bicyclic ring system which may be saturated, partially saturated or aromatic and may optionally have additional heteroatoms selected from O, N or S, the said ring system may further be optionally substituted;
R⁶ and R⁷, for each occurrence, are independently hydrogen, C₁₋₆alkyl, C₁₋₆ hydroxyalkyl, C₆₋₁₀aryl, C₆₋₁₀arylC₁₋₄alkyl, C₃₋₇cycloalkyl, heterocyclyl, heterocyclylC₁₋₄alkyl, heteroaryl or heteroarylC₁₋₄alkyl; or
R⁶ and R⁷ taken together may form a spiro, monocyclic or a bicyclic ring system which may be saturated or partially saturated and may optionally have additional heteroatoms selected from O, N or S, the said ring system may further be optionally substituted;
wherein when a group is optionally substituted, the substituents are selected from the group consisting of hydroxyl, cyano, nitro, C₁₋₆alkyl, C₂₋₆alkenyl, C₂₋₆alkynyl, C₁₋₆alkoxy, C₂₋₆alkenyloxy, C₂₋₆alkynyloxy, halogen, C₁₋₆haloalkyl, C₁₋₆perhaloalkyl, C₁₋₆₋alkylcarbonyl, (CH₂)ₙ—COOR³, amino, C₁₋₆₋alkylamino, di-C₁₋₆₋alkylamino, aminocarbonyl, C₁₋₆₋alkylaminocarbonyl, di-C₁₋₆₋alkylaminocarbonyl, C₁₋₆₋alkylcarbonylamino, C₁₋₆₋alkylcarbonyl(C₁₋₆₋alkyl)amino, C₁₋₆alkoxycarbonylamino, C₁₋₆₋alkylsulfonylamino, C₁₋₆₋alkylsulfonyl(C₁₋₆₋alkyl)amino, C₁₋₆alkylthio, C₁₋₆₋alkylsulfanyl, C₁₋₆₋alkylsulfonyl, aminosulfonyl, C₁₋₆₋alkylaminosulfonyl and di-C₁₋₆alkylaminosulfonyl, aminocarbonylC₁₋₆alkyl, C₁₋₆alkylaminocarbonylC₁₋₆alkyl, di-C₁₋₆alkylaminocarbonylC₁₋₆alkyl, sulfanylC₁₋₆alkyl, C₁₋₆alkylsulfanylC₁₋₆alkyl, sulfinylC₁₋₆alkyl, C₁₋₆alkylsulfinylC₁₋₆alkyl, sulfonylC₁₋₆alkyl, C₁₋₆alkylsulfonylC₁₋₆alkyl, cycloalkyl, C₆₋₁₀aryl (such as a phenyl), heterocyclyl, heteroaryl, heterocyclylcarbonyl, pyrrolidinocarbonyl, azetidinocarbonyl, cycloalkylcarbonylamino, cyclopropylcarbonylamino, cyclopentycarbonylamino, cyclohexylcarbonylamino, C₆₋₁₀arylcarbonylamino, and phenylcarbonylamino, wherein each of the aforementioned groups may be optionally substituted by one or more halogen, C₁₋₆alkyl, hydroxyl, oxo, C₁₋₆₋alkoxy, amino, C₁₋₆₋alkylamino, di-C₁₋₆₋alkylamino or cyano.

2. The compound according to claim 1, wherein the compound is represented by formula (II) or (III): 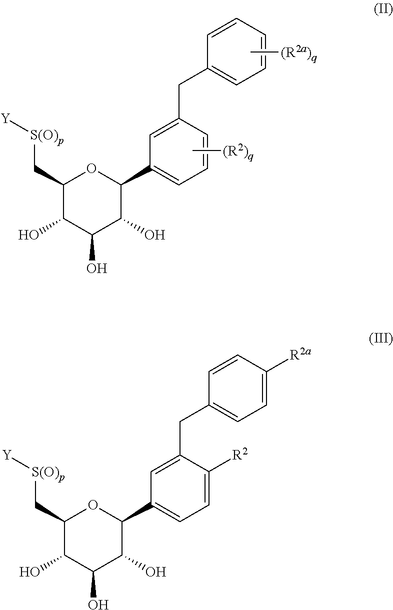
or a pharmaceutically acceptable salt thereof, wherein,
R² and R^{2a}, for each occurrence, are independently selected from halogen, hydroxy, C₁₋₄ hydroxylalkyl, cyano, —NR⁴R⁵, —CH₂NR⁴R⁵, C₁₋₄ alkyl, C₃₋₇ cycloalkyl, C₁₋₄ alkoxy, —S(O)ₚR³, —OS(O)ₚR³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₆₋₁₀aryloxy, heterocyclyl and heteroaryl;
p, for each occurrence, is independently 0, 1 or 2;
q, for each occurrence, is independently 1, 2, or 3; and
Y is optionally substituted C₆₋₁₀aryl or an optionally substituted heteroaryl.

3. The compound according to claim 1, wherein the compound is represented by formula (IV) or (V) 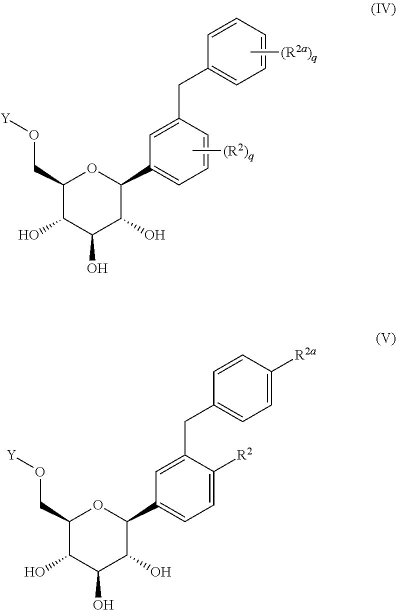
or a pharmaceutically acceptable salt thereof, wherein,
R² and R^{2a}, for each occurrence, are independently selected from halogen, hydroxy, C₁₋₄ hydroxylalkyl, cyano, —NR⁴R⁵, —CH₂NR⁴R⁵, C₁₋₄ alkyl, C₃₋₇ cycloalkyl, C₁₋₄ alkoxy, —S(O)ₚR³, —OS(O)ₚR³, —C(O)R³, —C(O)OR³, —CH₂C(O)OR³, —C(O)NR⁴R⁵, —CH₂C(O)NR⁴R⁵, —NR³C(O)NR⁴R⁵, —NR³C(O)OR³, C₁₋₆ haloalkyl, C₁₋₆ perhaloalkyl, C₆₋₁₀aryloxy, heterocyclyl and heteroaryl;
p, for each occurrence, is independently 0, 1 or 2;
q, for each occurrence, is independently 1, 2, or 3; and
Y is optionally substituted C₆₋₁₀aryl or an optionally substituted heteroaryl.

4. The compound according to claim 1, or a pharmaceutically acceptable salt thereof, wherein
Y is C₆₋₁₀aryl or heteroaryl.

5. The compound according to claim 1, or a pharmaceutically acceptable salt thereof, wherein Y is substituted phenyl.

6. The compound according to claim 1, or a pharmaceutically acceptable salt thereof, wherein
Y is 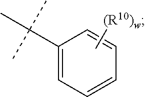
R¹⁰ is independently halogen, hydroxy, C₁₋₄ hydroxylalkyl, cyano, —NR¹⁶R¹⁷, oxo (═O), —CH₂NR¹⁶R¹⁷, C₁₋₄ alkyl, C₃₋₇ cycloalkyl, C₁₋₄ alkoxy, —S(O)ₚR¹⁸, —OS(O)₂R¹⁸, —C(O)R¹⁸, —C(O)OR¹⁸, —CH₂C(O)OR¹⁸, —C(O)NR¹⁶R¹⁷, —CH₂C(O)NR¹⁶R¹⁷, —NR¹⁸C(O)NR¹⁶R¹⁷, —NR¹⁸C(O)R¹⁸, —NR¹⁸C(O)OR¹⁸, —CH₂NR¹⁶C(O)OR¹⁸, —CH₂NR¹⁶C(O)NR¹⁶R¹⁷, —CH₂NR¹⁶S(O)ₚR¹⁸, —S(O)₂NR¹⁶R¹⁷, C₁₋₆ haloalkyl, C₁₋₆ perhaloalkyl, C₆₋₁₀aryloxy, heterocyclyl, heteroaryl;
R¹⁸ is hydrogen, C₁₋₆ alkyl, C₃₋₇ cycloalkyl, C₆₋₁₀aryl, heteroaryl, or heterocyclyl;
R¹⁶ and R¹⁷ are independently hydrogen, C₁₋₆ alkyl, C₃₋₇ cycloalkyl, C₆₋₁₀aryl(C₁₋₄)alkyl, C₆₋₁₀aryl, heteroaryl, heteroaryl(C₁₋₄)alkyl, heterocyclyl, heterocyclyl(C₁₋₄)alkyl or
R¹⁶ and R¹⁷ taken together may form a monocyclic or a bicyclic ring system which may be saturated, partially saturated or aromatic and may optionally have additional heteroatoms selected from O, N or S, the said ring system may further be optionally substituted;
p, for each occurrence, is independently 0, 1 or 2; and
w is 0, or an integer from 1-4.

7. The compound according to claim 6, or a pharmaceutically acceptable salt thereof, wherein Y is

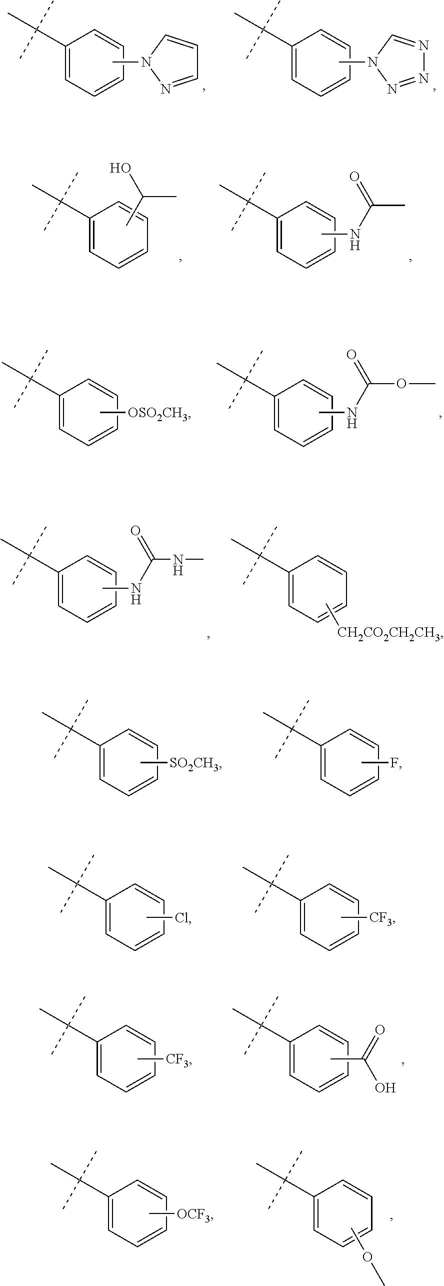

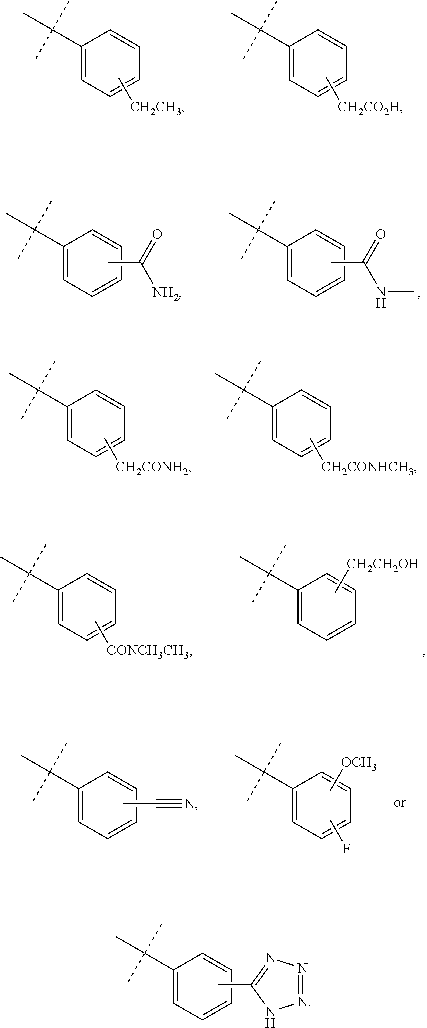

8. The compound according to claim 6, or a pharmaceutically acceptable salt thereof, wherein Y is

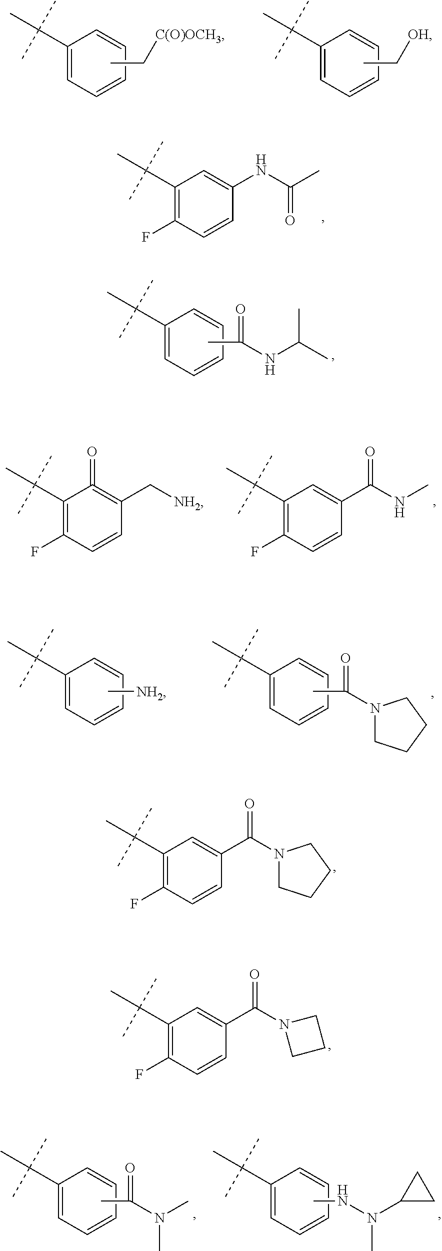

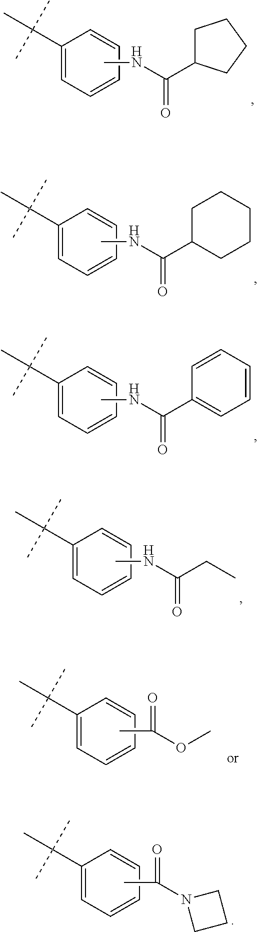

9. The compound according to claim 1, or a pharmaceutically acceptable salt thereof, wherein Y is

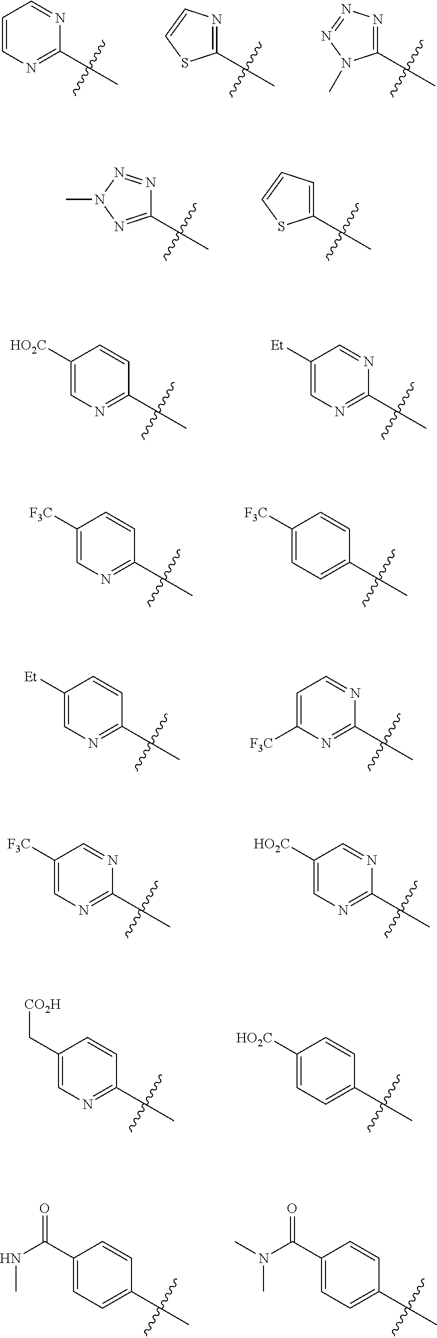

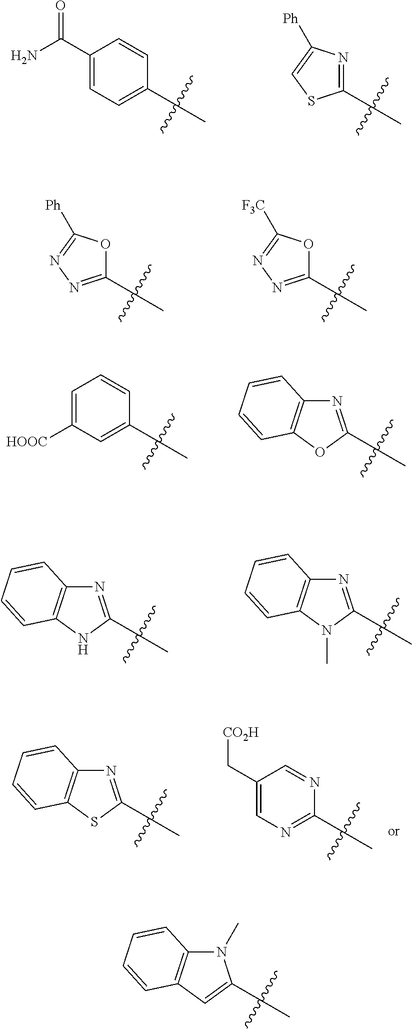

10. The compound according to claim 1, or a pharmaceutically acceptable salt thereof, wherein Y is

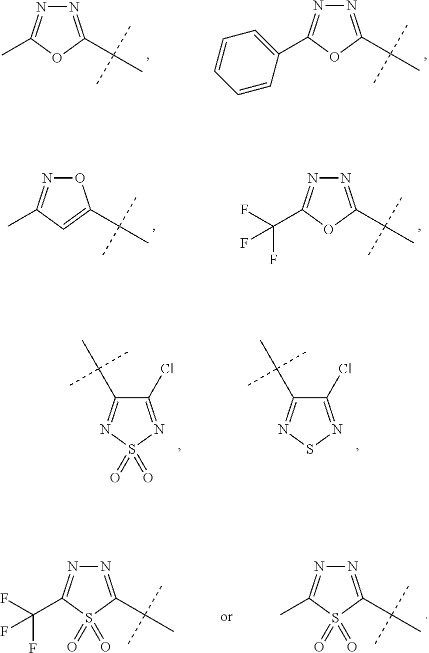

11. (canceled)

12. A pharmaceutical composition, comprising:
a compound according to claim 1, or a pharmaceutically acceptable salt thereof, and
a pharmaceutically acceptable excipient or carrier.

13. A method of treating diabetes, comprising:
administering a compound according to claim 1, or a pharmaceutically acceptable salt thereof, to a subject in need thereof.

14. A method of treating a disease or condition mediated by inhibition of sodium D-glucose cotransporter in a mammal, comprising:
administering to the mammal in need thereof a therapeutically effective amount of a compound according to claim 1, or a pharmaceutically acceptable salt thereof.

15. The method according to claim 14, wherein the disease or condition is metabolic syndrome, Syndrome X, diabetes, insulin resistance, decreased glucose tolerance, non-insulin-dependent diabetes mellitus, Type II diabetes, Type I diabetes, diabetic complications, a body weight disorder, weight loss, body mass index or a leptin related disease.

16. The method according to claim 15, wherein the metabolic syndrome is dyslipidemia, obesity, insulin resistance, hypertension, microalbuminemia, hyperuricaemia, or hypercoagulability.

17. A pharmaceutical composition comprising a therapeutically effective amount of the compound of claim 1, or a pharmaceutically acceptable salt thereof, in combination with a therapeutically effective amount of insulin, an insulin derivative, an insulin mimetic, an insulin secretagogue, an insulinotropic sulfonylurea receptor ligand, a PPAR ligand, an insulin sensitizer, a biguanide, an alpha-glucosidase inhibitor; GLP-1, a GLP-1 analog, a GLP-1 mimetic, a DPPIV inhibitor, an HMG-CoA reductase inhibitor, a squalene synthase inhibitor, an FXR ligand, an LXR ligand, cholestyramine, a fibrate, nicotinic acid, or aspirin.

18-20. (canceled)
